(19) United States
(12) Patent Application Publication
(10) Pub. No.: US 2026/0207322 A1
(43) Pub. Date: Jul. 23, 2026

(54) Systems and Methods for Autologous Regeneration of Tissue

(71) Applicant: Medline Industries, LP, Springfield, IL (US)

(72) Inventors: Marvin A. Guiles, Stow, MA (US); Aaron McPherson, Boston, MA (US); Thomas J. Evans, Bedford, MA (US); Carol Barnett, Springfield, IL (US); Meng Chen, Springfield, IL (US)

(21) Appl. No.: 19/030,853

(22) Filed: Jan. 17, 2025

(51) Int. Cl. A61F 2/10; A61B 17/00; A61B 17/32; A61B 17/3205; A61B 90/00

(52) U.S. Cl. CPC A61F 2/105; A61B 17/32053; A61B 90/361; A61B 2017/00398; A61B 2017/00734; A61B 2017/00747; A61B 2017/00969; A61B 2017/320064; A61B 2090/373

(57) ABSTRACT

A system can include a plurality of hollow tubes, each hollow tube can be configured to harvest a respective tissue portion from a tissue site. The respective tissue portion can include skin tissue. The system can include an actuation system configured to translate the plurality of hollow tubes simultaneously into the tissue site. The system can include a rotational drive configured to rotate each hollow tube of the plurality of tubes. In some embodiments, as the actuation system translates the plurality of hollow tubes into the tissue site, the rotational drive can be configured to rotate each hollow tube of the plurality of hollow tubes, or after the actuation system translates the plurality of hollow tubes into the tissue site, the rotational drive can be configured to rotate each hollow tube of the plurality of hollow tubes to ensure each tissue portion is severed from the tissue site.

### BACKGROUND OF THE INVENTION

The subject matter disclosed herein generally relates to a skin grafting system and, more particularly, to a system that can include a device for harvesting and scattering skin microcolumns.

An autograft can refer to tissue transplanted from one part of an individual's body (e.g., a “donor site”) to another part (e.g., a “recipient site”). Autografts can be used, for example, to replace missing skin and other tissue, or to accelerate healing resulting from trauma, wounds, burns, surgery and birth defects. Availability of tissue for autografting can be limited by characteristics of candidate donor sites, including a number or total area of tissue grafts, healing behavior of the donor site, similarity of the donor and recipient sites, aesthetic considerations, and the like.

Skin grafting can be performed surgically. For example, a comparative autograft procedure can include excision or surgical removal of burn injured tissue, choosing a donor site, which can be an area from which healthy skin is removed to be used as cover for the cleaned burned area, and harvesting, where the graft can be removed from the donor site (e.g., using an instrument similar to an electric shaver). Such instrument (e.g., a dermatome) can be structured to gently shave a thin piece of tissue (e.g., about 10/1000 of an inch thick for a split-thickness graft) from the skin at the undamaged donor site to use as a skin graft. The skin graft can then be placed over the cleaned wound to heal. Donor skin tissue can be removed to such a depth that the donor site can heal on its own, in a process similar to that of healing of a second degree burn.

Traditionally, sheet grafts and meshed grafts are the two types of autografts often used for a permanent wound coverage. A sheet graft can refer to a piece of skin tissue removed from an undamaged donor site of the body, in a process that can be referred to as harvesting. The size of the donor skin piece that is used can be about the same size as the damaged area. The sheet graft can be applied over the excised wound, and stapled or otherwise fastened in place. The donor skin tissue used in sheet grafts may not stretch significantly, and a sheet graft can be obtained that is slightly larger than the damaged area to be covered because there can often be a slight shrinkage of the graft tissue after harvesting.

Sheet grafts can provide an improved appearance of the repaired tissue site. For example, sheet grafts can be used on large areas of the face, neck and hands if they are damaged, so that these more visible parts of the body can appear less scarred after healing. A sheet graft can be used to cover an entire burned or damaged region of skin. Small areas of a sheet graft can be lost after placement because a buildup of fluid (e.g., a hematoma) can occur under the sheet graft following placement the sheet graft.

A meshed skin graft can be used to cover larger areas of open wounds that can be difficult to cover using sheet grafts. Meshing of a skin graft can facilitate skin tissue from a donor site to be expanded to cover a larger area. It also can facilitate draining of blood and body fluids from under the skin grafts when they are placed on a wound, which may help prevent graft loss. The expansion ratio (e.g., a ratio of the unstretched graft area to the stretched graft area) of a meshed graft may typically be between about 1:1 to 1:4. For example, donor skin can be meshed at a ratio of about 1:1 or 1:2 ratio, whereas larger expansion ratios may lead to a more fragile graft, scarring of the meshed graft as it heals, or extended healing times.

A comparative graft meshing procedure can include running the donor skin tissue through a machine that cuts slits through the tissue, which can facilitate the expansion in a pattern similar to that of fish netting or a chain-link fence. Healing can occur as the spaces between the mesh of the stretched graft, which can be referred to as gaps or interstices, fill in with new epithelial skin growth. However, meshed grafts can be less durable graft than sheet grafts, and a large mesh can lead to permanent scarring after the graft heals.

As an alternative to autografting, skin tissue obtained from recently deceased people (which can be referred to, e.g. as a homograft, an allograft, or cadaver skin) can be used as a temporary cover for a wound area that has been cleaned. Unmeshed cadaver skin can be put over the excised wound and stapled in place. Post-operatively, the cadaver skin can be covered with a dressing. Wound coverage using cadaveric allograft can then be removed prior to permanent autografting.

A xenograft or heterograft can refer to skin taken from one of a variety of animals, for example, a pig. Heterograft skin tissue can also be used for temporary coverage of an excised wound prior to placement of a more permanent autograft, and can be used because of a limited availability or high expense of human skin tissue. In some cases religious, financial, or cultural objections to the use of human cadaver skin may also be factors leading to use of a heterograft. Wound coverage using a xenograft or an allograft is generally a temporary procedure which can be used until harvesting and placement of an autograft is feasible.

Harvesting of the graft tissue from the donor site can generally generate undesirable large-scale tissue damage to the donor site. On the other hand, small areas of skin wounding adjacent to healthy tissue can be well-tolerated, and may heal quickly. Such healing of small wounds can occur in techniques such as “fractional photothermolysis” or “fractional resurfacing,” in which patterns of damage having a small dimension can be created in skin tissue. These exemplary techniques are described, for example, in U.S. Pat. No. 6,997,923. Small-scale damage patterns can heal quickly by regrowth of healthy tissue, and can further provide desirable effects such as skin tightening without visible scarring.

In some configurations, there can be problems with conventional skin grafting systems. Therefore, it would be desirable to have improved systems and methods for autologous regeneration of tissue, and particularly skin tissue.

### BRIEF DESCRIPTION OF THE DISCLOSURE

According to one aspect of the present disclosure, a skin grafting system is provided. The system comprises a handheld device comprising a device housing forming an interior that secures a drive system; a cartridge comprising a plurality of hollow microneedles surrounded by a peripheral housing and configured to be operated by the drive system to extend and retract past the peripheral housing into a subject to harvest tissue during a skin grafting process; and a feedback system comprising a user interface configured to provide information to a user of the skin grafting system.

The following description and the accompanying drawings set forth in detail certain illustrative non-limiting examples of the present disclosure. However, these non-limiting examples are indicative of but a few of the various ways in which the principles of the disclosure can be employed. Other non-limiting examples and features will become apparent from the following detailed description of the present disclosure when considered in conjunction with the drawings.

Some non-limiting examples of the disclosure provide a skin grafting system. The system can include a handheld device comprising a device housing forming an interior that secures a drive system, a cartridge comprising a plurality of hollow microneedles surrounded by a peripheral housing and configured to be operated by the drive system to extend and retract past the peripheral housing into a subject to harvest tissue during a skin grafting process, and a feedback system comprising a user interface configured to provide information to a user of the skin grafting system.

In some non-limiting examples, a plurality of hollow microneedles can be a plurality of hypotubes with substantially blunt tips.

In some non-limiting examples, a system can include a rotation mechanism configured to cause at least one of the plurality of hollow microneedles to rotate about its central axis.

In some non-limiting examples, a rotation mechanism can include a needle drive and a belt frictionally engaged with a row of the plurality of hollow microneedles to translate a rotational movement of the needle drive into a rotational movement of each microneedle of the row of the plurality of microneedles.

In some non-limiting examples, a drive system can include an ultrasonic actuator.

In some non-limiting examples, a drive system can include at least one reloadable spring.

In some non-limiting examples, a system can include a power module configured to provide power to the handheld device.

In some non-limiting examples, a power module can include a rechargeable battery pack.

In some non-limiting examples, a user interface can include a display on a surface of the handheld device.

In some non-limiting examples, a display can be a touchscreen display.

In some non-limiting examples, a display can be configured to display an input request to a user of a system to display an output response to the user. The output response can be based on information received in response to the input request.

In some non-limiting examples, a system can include an image sensor configured to image a target area of a skin grafting process.

In some non-limiting examples, a user interface can be configured to show a real-time image of a target area as captured by an image sensor.

In some non-limiting examples, a cartridge can be removably attached to a device housing.

In some non-limiting examples, a cartridge can be configured to communicate with a handheld device via an interface.

In some non-limiting examples, an interface can include a wireless communication interface.

In some non-limiting examples, an interface can include an optical communication interface.

Some embodiments of the disclosure provide a system. The system can include a first plurality of hollow tubes, each hollow tube of the first plurality of hollow tubes can be configured to harvest a respective tissue portion from a tissue site. The respective tissue portion can include skin tissue. The system can include a second plurality of hollow tubes, each hollow tube of the second plurality of hollow tubes can be configured to harvest a respective tissue portion from the tissue site. The respective tissue portion can include skin tissue. The system can include an actuation system configured to independently translate the first plurality of hollow tubes and the second plurality of hollow tubes. The system can include an ultrasonic transducer configured to vibrate at least one hollow tube of the first plurality of hollow tubes or the second plurality of hollow tubes at an ultrasonic frequency during translation of the first plurality of hollow tubes into the tissue site.

In some embodiments, the system can include a heat sink coupled to the at least one hollow tube to cool the hollow tube after application of an ultrasonic frequency vibration, or a cooling system thermally coupled to the at least one hollow tube. Thee cooling system can include a refrigerant configured to absorb heat generated from the application of the ultrasonic frequency vibration.

Some embodiments of the disclosure provide a system. The system can include a first plurality of hollow tubes, each hollow tube of the first plurality of hollow tubes can be configured to harvest a respective tissue portion from a tissue site. The respective tissue portion can include skin tissue. The system can include a second plurality of hollow tubes, each hollow tube of the second plurality of hollow tubes can be configured to harvest a respective tissue portion from the tissue site. The respective tissue portion can include skin tissue. The system can include an actuation system configured to independently translate the first plurality of hollow tubes and the second plurality of hollow tubes. The system can include one or more computing devices that can be configured to cause the actuation system to translate the first plurality of hollow tubes into the tissue site, determine that that first plurality of hollow tubes has been translated after causing the actuation system to translate the first plurality of hollow tubes, cause the actuation system to translate the second plurality of hollow tubes into the tissue site, and determine that that second plurality of hollow tubes has been translated after causing the actuation system to translate the second plurality of hollow tubes.

In some embodiments, the system can include a sensor configured to detect a position of a first plurality of hollow tubes. In some embodiments, determining that the first plurality of hollow tubes has been translated after causing an actuation system to translate the first plurality of hollow tubes includes one or more computing devices receiving from the sensor, sensor data indicative of the absence of the first plurality of hollow tubes, and based on the sensor data, determining that the first plurality of tubes has been translated.

In some embodiments, one or more computing devices can be further configured to determine that all hollow tubes have been translated into a tissue site, based on at least one of the determinations that a first plurality of hollow tubes or a second plurality of hollow tubes have been translated.

Some embodiments of the disclosure provide a system. The system can include a first plurality of hollow tubes, each hollow tube of the first plurality of hollow tubes can be configured to harvest a respective tissue portion from a tissue site. The respective tissue portion can include skin tissue. The system can include a second plurality of hollow tubes, each hollow tube of the second plurality of hollow tubes can be configured to harvest a respective tissue portion from the tissue site. The respective tissue portion can include skin tissue. The system can include an actuation system configured to independently translate the first plurality of hollow tubes and the second plurality of hollow tubes. The actuation system can include an actuator that emits sound having a first sound wave during movement of the actuator. The system can include a speaker that emits a second sound wave during movement of the actuator that is at least partially inverted relative to the first sound wave thereby mitigating the amplitude of the first sound wave to at least partially cancel the noise from the first sound wave.

In some embodiments, an actuator is a solenoid.

In some embodiments, the system can include a housing, a microphone coupled to the housing and configured to acquire acoustic information indicative of the first sound wave during movement of an actuator, and one or more computing devices in communication with the microphone and the speaker. The one or more computing devices can be configured to acquire the acoustic information indicative of the first sound wave during movement of the actuator, and using the acoustic information, cause the speaker to emit the second sound wave during movement of the actuator.

In some embodiments, using the acoustic information can include one or more computing devices inverting the acoustic information or shifting the acoustic information to create a second sound wave.

Some embodiments of the disclosure provide a system. The system can include a first plurality of hollow tubes, each hollow tube of the first plurality of hollow tubes can be configured to harvest a respective tissue portion from a tissue site. The respective tissue portion can include skin tissue. The system can include a second plurality of hollow tubes, each hollow tube of the second plurality of hollow tubes can be configured to harvest a respective tissue portion from the tissue site. The respective tissue portion can include skin tissue. The system can include a peripheral housing that surrounds the first plurality of hollow tubes and the second plurality of hollow tubes, a stabilizer, and a spring coupled to the stabilizer. The spring can be configured to force the stabilizer against the tissue site to stabilize the tissue site during insertion of the first plurality of tubes or the second plurality of tubes into the tissue site.

In some embodiments, the stabilizer can be positioned between a first plurality of hollow tubes or a second plurality of hollow tubes, the stabilizer can be positioned to a first side of the peripheral housing, such that the stabilizer is closer to the first side of the peripheral housing than the first plurality of hollow tubes and the second plurality of hollow tubes are to a second opposite side of the peripheral housing, or the stabilizer can be flush with a side of the peripheral housing and located within the peripheral housing.

In some embodiments, when the stabilizer is pressed against the tissue site, the stabilizer can bias the spring to provide a consistent force to the tissue site thereby stabilizing the tissue site.

Some embodiments of the disclosure provide a system. The system can include a housing, and a first cartridge removably coupled to the housing. The first cartridge can include a first plurality of hollow tubes, each hollow tube of the first plurality of hollow tubes can be configured to harvest a respective tissue portion from a tissue site. The respective tissue portion can include skin tissue. The system can include a second cartridge removably coupled to the housing. The second cartridge can include a second plurality of hollow tubes, each hollow tube of the second plurality of hollow tubes can be configured to harvest a respective tissue portion from a tissue site. The respective tissue portion can include skin tissue. The first cartridge can be different than the second cartridge.

In some embodiments, a second plurality of hollow tubes can be less than a first plurality of hollow tubes.

In some embodiments, a first cartridge can include a first peripheral housing. The first peripheral housing can surround a first plurality of hollow tubes. The first peripheral housing can define a first area. The second cartridge can include a second peripheral housing. The second peripheral housing can surround a second plurality of hollow tubes. The second peripheral housing can define a second area. The first area can be substantially the same as the second area.

In some embodiments, at least one hollow tube of a first plurality of hollow tubes can be longer than at least one hollow tube of a second plurality of hollow tubes.

In some embodiments, an inner width of at least one hollow tube of a first plurality of hollow tubes can be larger than an inner width of at least one hollow tube of a second plurality of hollow tubes.

Some embodiments of the disclosure provide a system. The system can include a plurality of hollow tubes, each hollow tube of the plurality of hollow tubes can be configured to harvest a respective tissue portion from a tissue site. The respective tissue portion can include skin tissue. The system can include an actuation system configured to translate the plurality of hollow tubes simultaneously into the tissue site. The system can include a rotational drive configured to rotate each hollow tube of the plurality of tubes. In some embodiments, as the actuation system translates the plurality of hollow tubes into the tissue site, the rotational drive can be configured to rotate each hollow tube of the plurality of hollow tubes, or after the actuation system translates the plurality of hollow tubes into the tissue site, the rotational drive can be configured to rotate each hollow tube of the plurality of hollow tubes to ensure each tissue portion is severed from the tissue site.

In some embodiments, a rotational drive can rotate each hollow tube of the plurality of hollow tubes about a longitudinal axis of the respective hollow tube.

In some embodiments, a plurality of hollow tubes can be a first plurality of hollow tubes. The system can include a second plurality of hollow tubes. An actuation system can be configured to independently translate the first plurality of hollow tubes and the second plurality of hollow tubes. A rotational drive can be configured to rotate each hollow tube of the second plurality of hollow tubes.

In some embodiments, a system can include an array of hollow tubes that can include a first plurality of hollow tubes and a second plurality of hollow tubes.

In some embodiments, a first plurality of hollow tubes can be a first row of hollow tubes. A second plurality of hollow tubes can be a second row of hollow tubes.

In some embodiments, a rotational drive can be at least one of a belt or a chain.

In some embodiments, an inner width of each hollow tube of a plurality of hollow tubes can be less than or equal to 1 millimeter, or the inner width of each hollow tube of the plurality of hollow tubes can be less than or equal to 0.6 millimeters.

Some embodiments of the disclosure provide a system. The system can include a first plurality of hollow tubes, each hollow tube of the first plurality of hollow tubes can be configured to harvest a first respective tissue portion from a tissue site. The first respective tissue portion can include skin tissue. The system can include a second plurality of hollow tubes, each hollow tube of the second plurality of hollow tubes can be configured to harvest a second respective tissue portion from the tissue site. The second respective tissue portion can include skin tissue. The system can include an actuation system configured to independently translate the first plurality of hollow tubes and the second plurality of hollow tubes. The actuation system can include a spring, a plunger coupled to the spring, a motor coupled to the plunger and configured to load the spring, and a brake configured to lock the plunger to prevent unloading of the spring. The brake can be configured to release the plunger to allow the spring to unload and drive the plunger thereby forcing the first plurality of hollow tubes into the tissue site.

In some embodiments, a motor can be configured to move a plunger back into a first position thereby reloading a spring. When the plunger is in the first position, the brake can be configured to lock the plunger into the locked position. When the plunger is in the first position with the spring reloaded, the brake can be configured to release the plunger thereby unloading the spring to force the second plurality of hollow tubes into the tissue site.

In some embodiments, a system can include a housing, and a battery pack electrically coupled to the actuation system and removably coupled to the housing. A motor can be a DC motor.

Some embodiments of the disclosure provide a housing, and a plurality of hollow tubes, each hollow tube of the plurality of hollow tubes can be configured to harvest a respective tissue portion from a tissue site. The respective tissue portion can include skin tissue. The system can include an image sensor coupled to the housing and configured to acquire one or more images of the tissue site or other surrounding area. The system can include a display, and one or more computing devices in communication with the image sensor and the display. The one or more computing devices can be configured to acquire, using the image sensor, an image of at least a portion of the tissue site, and cause the display to present the image of the portion of the tissue site.

In some embodiments, a plurality of hollow tubes can be a first plurality of hollow tubes. The system can include a second plurality of hollow tubes, each hollow tube of the second plurality of hollow tubes can be configured to harvest a respective tissue portion from the tissue site that includes skin tissue. The system can include an actuation system configured to independently translate the first plurality of hollow tubes and the second plurality of hollow tubes.

In some embodiments, at least one of a display can be coupled to a housing, an image sensor can be coupled to a first end of the housing opposite a second end of the housing that includes a handle, or one or more computing devices can be further configured to shift an image of the at least a portion of the tissue site, based on a distance between the image sensor and a center of a plurality of hollow tubes.

Some embodiments of the disclosure provide a system. The system can include a first plurality of hollow tubes, each hollow tube of the first plurality of hollow tubes can be configured to harvest a first respective tissue portion from a tissue site. The first respective tissue portion can include skin tissue. The system can include a second plurality of hollow tubes, each hollow tube of the second plurality of hollow tubes can be configured to harvest a second respective tissue portion from the tissue site. The second respective tissue portion can include skin tissue. The system can include a peripheral housing that surrounds the first plurality of hollow tubes and the second plurality of hollow tubes. The system can include a stabilizer coupled to the peripheral housing. The stabilizer can separate the first plurality of hollow tubes from the second plurality of hollow tubes. The peripheral housing and the stabilizer can be configured to be forced against the tissue site to stabilize the tissue site during insertion of the first plurality of hollow tubes or the second plurality of hollow tubes into the tissue site.

In some embodiments, a system can include an actuation system configured to independently translate a first plurality of hollow tubes and a second plurality of hollow tubes.

In some embodiments, a stabilizer can be a first stabilizer. The system can include a third plurality of hollow tubes, each hollow tube of the third plurality of hollow tubes can be configured to harvest a third respective tissue portion from a tissue site that can include skin tissue. The system can include a second stabilizer coupled to the peripheral housing. The second stabilizer can separate a second plurality of hollow tubes from the third plurality of hollow tubes.

In some embodiments, a system can include a device housing and a cartridge removably coupled to the device housing. The cartridge can include a peripheral housing, a stabilizer, a first plurality of hollow tubes, and a second plurality of hollow tubes.

In some embodiments, a stabilizer is rigid. The stabilizer can be substantially flat. When the stabilizer is forced against the tissue site, the tissue site can flatten against the stabilizer.

Some embodiments of the disclosure provide a system. The system can include a housing and a plurality of hollow tubes, each hollow tube of the plurality of hollow tubes can be configured to harvest a respective tissue portion from a tissue site and subsequently dispense the respective tissue portion onto a target site. The respective tissue portion can include skin tissue. The system can include an image sensor coupled to the housing and configured to acquire one or more images of the tissue site, a light source coupled to the housing, and one or more computing devices in communication with the image sensor and the light source. The one or more computing devices can be configured to at least one of cause the light source to illuminate the target site during acquisition of an image of the target site by the image sensor, or cause the light source to project an illumination pattern at the target site. The position of the illumination pattern corresponding with a scattering location of the tissue portions from the plurality of hollow tubes onto the tissue site.

Some embodiments of the disclosure provide a system. The system can include a housing, and a plurality of hollow tubes, each hollow tube of the plurality of hollow tubes can be configured to harvest a respective tissue portion from a tissue site and subsequently dispense the respective tissue portion onto a target site. The respective tissue portion can include skin tissue. The system can include a scattering system configured to dispense the tissue portion from each hollow tube of the plurality of hollow tubes onto the target site, and a substrate coupled to or removably coupled to the housing. The substate can be adjustable to adjust a distance between the plurality of hollow tubes and the target site. A portion of the substate can contact the target site when the scattering system dispenses the portions of tissue from the plurality of hollow tubes.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The descriptions hereafter are provided with reference to the accompanying drawings, wherein like reference numerals denote like elements.

The following drawings are provided to help illustrate various features of non-limiting examples of the disclosure, and are not intended to limit the scope of the disclosure or exclude alternative implementations.

FIG. 1 is a top perspective view of a skin grafting system in accordance with some implementations of the present disclosure.

FIG. 2A is a front perspective view of the system of FIG. 1.

FIG. 2B is a top view of a user interface that can be included in the system of FIG. 2A, in accordance with some implementations of the present disclosure.

FIG. 3A is a cutaway view of the handheld device of FIG. 2A, in accordance with some implementations of the present disclosure.

FIG. 3B is a cutaway view of a housing corresponding to the handheld device of FIG. 2A, in accordance with some implementations of the present disclosure.

FIG. 4A is a front perspective view of an internal drive assembly and related elements corresponding to the handheld device of FIG. 2A, in accordance with some implementations of the present disclosure.

FIG. 4B is a front perspective view of a left frame assembly corresponding to the internal assembly of FIG. 4A, in accordance with some implementations of the present disclosure.

FIG. 4C is a front perspective view of a right frame assembly corresponding to the internal assembly of FIG. 4A, in accordance with some implementations of the present disclosure.

FIG. 4D is a front perspective view of a horizontal component assembly corresponding to the internal assembly of FIG. 4A, in accordance with some implementations of the present disclosure.

FIG. 4E is a front perspective view of a vertical component assembly corresponding to the internal assembly of FIG. 4A, in accordance with some implementations of the present disclosure.

FIG. 5A is a top perspective view of a cartridge assembly, in accordance with some implementations of the present disclosure.

FIG. 5B is a front perspective view of a cartridge corresponding to the cartridge of FIG. 5A, in accordance with some implementations of the present disclosure.

FIG. 6A is a front view of a microneedle that can harvest tissue, in accordance with some implementations of the present disclosure.

FIG. 6B is a front view of a microneedle that can harvest tissue, in accordance with some implementations of the present disclosure.

FIG. 6C is a front view of a microneedle array, in accordance with some implementations of the present disclosure.

FIG. 7 is a flowchart illustrating a method of harvesting and scattering tissue, in accordance with some implementations of the present disclosure.

FIG. 8 shows a schematic illustration of a general configuration of a system, which can be used for skin grafting.

FIG. 9 shows an example of a system for skin grafting and is a specific implementation of the system of FIG. 8.

FIG. 10 shows an example of a hollow tube.

FIG. 11 shows a graph illustration the relationship between the impulse and the distance the needle travels into the tissue.

FIG. 12A shows a spring coupled to a support, and a driver.

FIG. 12B shows a driver in a second position opposite the first position as illustrated in FIG. 12A.

FIG. 13 shows an example of a system, which can be a specific implementation of the system of FIG. 8.

FIG. 14A shows an example of a rotator.

FIG. 14B shows an example of another rotator.

FIG. 15A shows a rotator with a drive, and rotational couplers relative to a plurality of hollow tubes in segments.

FIG. 15B shows an example of a rotator with a drive and a rotational coupler, relative to a plurality of hollow tubes.

FIG. 16A shows an example of a simplified illustration of a system, which can be a skin grafting system, showing a specific implementation of a rotator.

FIG. 16B shows an example of a hollow tube without a rotator shown, but demonstrating another configuration of translation and rotation of a hollow tube.

FIGS. 17A-C show an example of another system, with components of this system being in different positions relative to the FIGS. 17A-C.

FIGS. 18A-C show an example of another system, again, with components of this system being in different positions relative to FIGS. 18A-C.

FIG. 19 shows an example of an actuation system.

FIG. 20 shows an example of a system.

FIG. 21 shows an example of a cooling system relative to hollow tubes.

FIG. 22A-C show an example of a detection system.

FIGS. 23A and 23B show an example of a detection system.

FIG. 24 shows a schematic illustration of an example of a system.

FIG. 25 shows another schematic illustration of the system of FIG. 24.

FIG. 26A shows a schematic illustration of the system of FIG. 24 positioned above a tissue site prior to insertion of the plurality of hollow tubes into the tissue.

FIG. 26B shows a cross-sectional view of the cartridge of the system of FIG. 26A surrounded by the tissue.

FIG. 27A shows a schematic illustration of the system of FIG. 26A positioned above the tissue site with a different cartridge.

FIG. 27B shows a schematic illustration of the system of FIG. 26A positioned on the tissue site with the different cartridge.

FIG. 27C shows a cross-sectional view of the different cartridge of FIG. 27B surrounded by the tissue.

FIG. 28A shows a schematic illustration of an example of a system.

FIG. 28B shows a schematic illustration of the system of FIG. 28A when raised above the recipient site.

FIG. 29A shows a schematic illustration of an example of a system.

FIG. 29B shows a schematic illustration of the system of FIG. 29A with a scatter aid to capture tissue portions before applying them onto a recipient site.

FIG. 30 shows the system of FIG. 29A with a different scatter aid, to help direct tissue portions during a scattering process.

FIG. 31 shows a schematic illustration of an example of a system.

FIG. 32A shows one example of an interaction between the display device and a user, while FIG. 32B shows another example of an interaction between the display device and the user.

FIGS. 33A-33D show schematic illustrations of different cartridges that illustrate each cartridge having one or more different characteristics or parameters.

FIG. 34 shows a flowchart of a process that can be implemented using any of the systems, devices, etc., described herein.

FIG. 35 shows a flowchart of another process that can be implemented using any of the systems, devices, etc., described herein.

### DETAILED DESCRIPTION OF THE DISCLOSURE

The following discussion is presented to enable a person skilled in the art to make and use the systems and methods of the present disclosure. Various modifications to the illustrated non-limiting examples will be readily apparent to those skilled in the art, and the high-level principles herein can be applied to other non-limiting examples and applications without departing from non-limiting examples of the present disclosure. Thus, non-limiting examples of the present disclosure are not intended to be limited to non-limiting examples shown, but are to be accorded the widest scope consistent with the principles and features disclosed herein.

The detailed description is to be read with reference to the figures. The figures depict selected non-limiting examples and are not intended to limit the scope of non-limiting examples of the present disclosure. Skilled artisans will recognize the examples provided herein have many useful alternatives and fall within the scope of non-limiting examples of the present disclosure. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

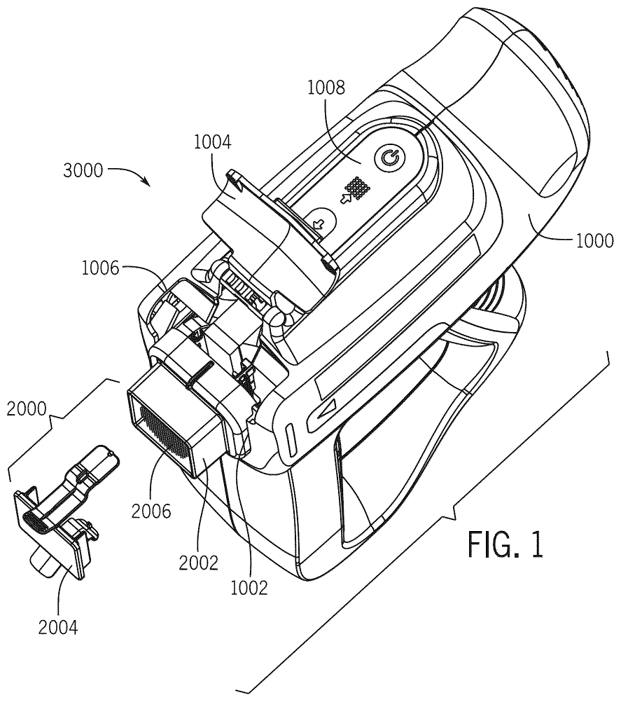

Referring now to FIG. 1, a skin grafting system 3000 is shown, in accordance with some implementations of the present disclosure. In some configurations, the skin grafting system 3000 can be configured to harvest and scatter donor tissue (e.g., direct harvested micrografts at a target site, such as a wound site thereby “seeding” the target site with harvested micrografts). As shown, the skin grafting system 3000 can include a handheld device 1000 and a cartridge assembly 2000. As will be described in greater detail below, the cartridge assembly 2000 can include a cartridge 2002 and a cartridge cover 2004. The cartridge 2002 can include a microneedle array 2006, according to some configurations.

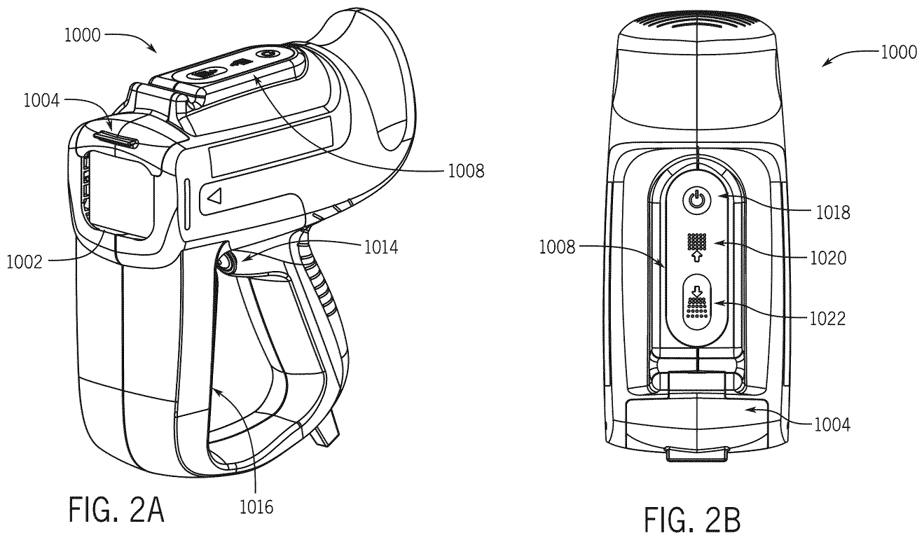

As shown by FIGS. 1-2B, the handheld device 1000 can include an engagement slot 1002 configured to receive the cartridge assembly 2000. A loading door 1004 can move between an “open” position (see, e.g., FIG. 1) and a “closed” position (see, e.g., FIGS. 2A-2B). In some configurations, the loading door 1004 can be hinged and further configured to open and close over a loading aperture 1006. The handheld device 1000 can include a door sensor, which can determine the position of the loading door 1004. The loading aperture 1006 can be sized such that the cartridge assembly 2000 can slide in and out of the engagement slot 1002, as desired by the user. Advantageously, the cartridge assembly 2000 can be single-use or disposable (including, for example, multiple uses for a single patient), while the handheld device 1000 can be designed to be multi-use. As shown by FIG. 2A, the handheld device 1000 can further include a trigger 1014. The trigger 1014 can be configured to activate a harvesting process or a scattering process in response to selection via a user interface 1008 or trigger inputs by a user. In some configurations, the handheld device 1000 can include an indicator light 1016. The indicator light 1016 can be positioned opposite the trigger 1014, such that a user can readily view the indicator light 1016 during harvesting or scattering.

In some configurations, the handheld device 1000 can include a user interface 1008. As shown, the user interface 1008 can include a stand-by input 1018, an indicator light 1020, or a scatter input 1022. In some configurations, the indicator light 1020 can operate the same as, or similar to, the indicator light 1016 (as described above). The stand-by input 1018, the indicator lights 1016 and 1020, and the scatter input 1022 can provide visual feedback to a user that correspond to current operation of the skin grafting system 3000 as the skin grafting system 3000 is utilized according to a skin grafting process, such as will be described.

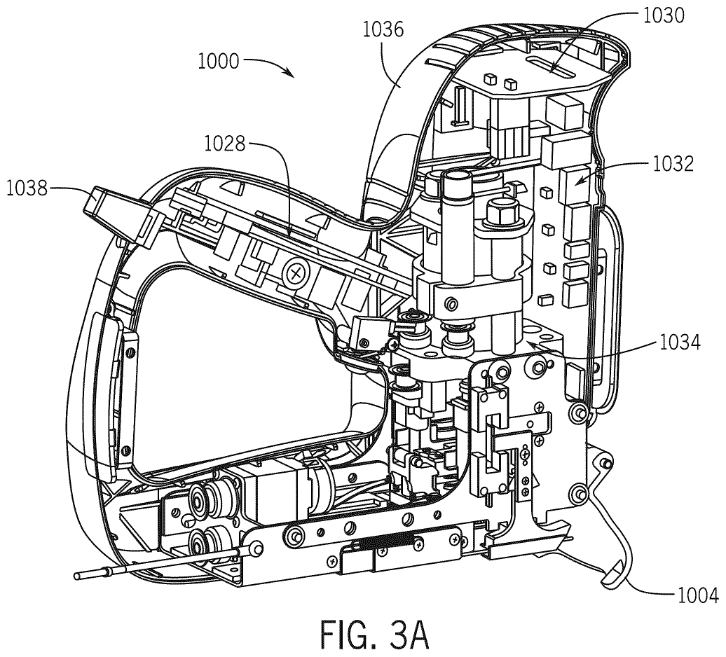

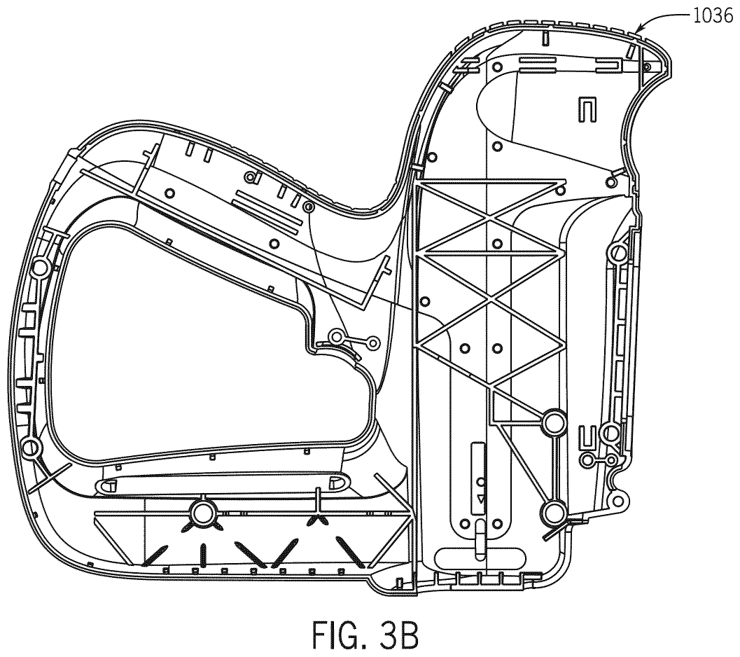

Referring now to FIGS. 3A-3B, cutaway views of the handheld device 1000 are shown, according to configurations of the present disclosure. The handheld device 1000 is shown to include various internal controllers. In some configurations, the handheld device 1000 can include a power module 1028, a solenoid controller 1030, or a main controller 1032. The power module 1028 can be in electrical communication with a power input 1038. In some configurations, a drive system 1034 can include a solenoid in communication with the solenoid controller 1030.

Still referring to FIGS. 3A-3B, in some configurations, the handheld device 1000 can include a housing 1036. The housing 1036 can include a left enclosure half and a right enclosure half. In some configurations, each of the left enclosure half, the right enclosure half, the loading door 1004 and the enclosure mount cover can be individually injection molded. The left and right enclosure halves can be made up of a hard plastic substrate, and in some configurations, a softer elastomeric over-molded section. Similarly, the loading door 1004 and the enclosure mount cover can be made up of hard plastic substrate. In some configurations, the interior of the housing 1036 can interface with internal subassemblies. As an example, ribs can be affixed to the interior of the housing 1036, and can be configured to support various printed circuit boards (PCBs). The ribs can separate the PCBs (e.g., power module 1028, solenoid controller 1030, and main controller 1032) from internal moving components. Additionally, in some configurations, the housing 1036 can support the user interface 1008 via pins and vibration damping boots. This can isolate the user interface 1008 from operational impacts (e.g., from a user, from internal moving components).

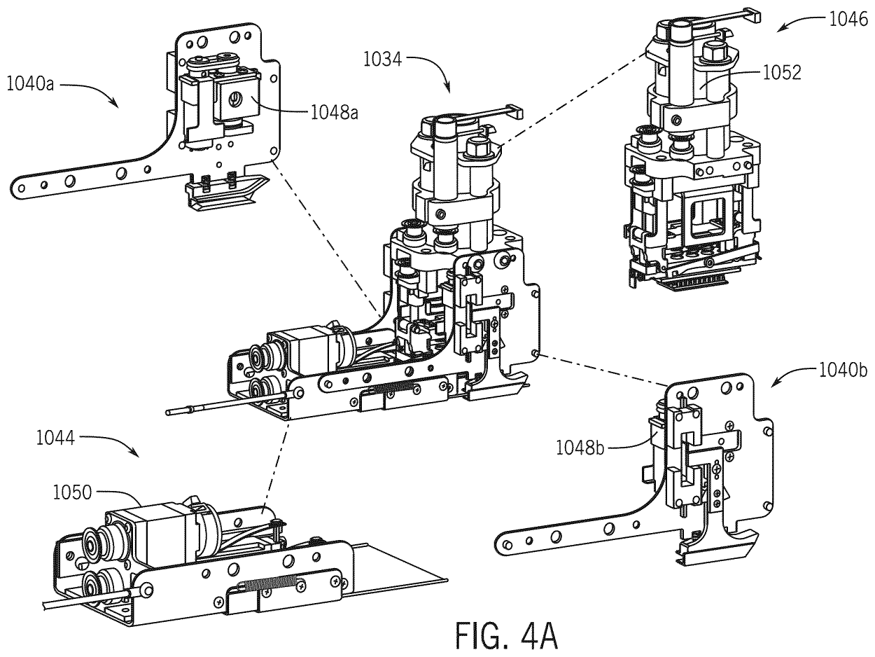

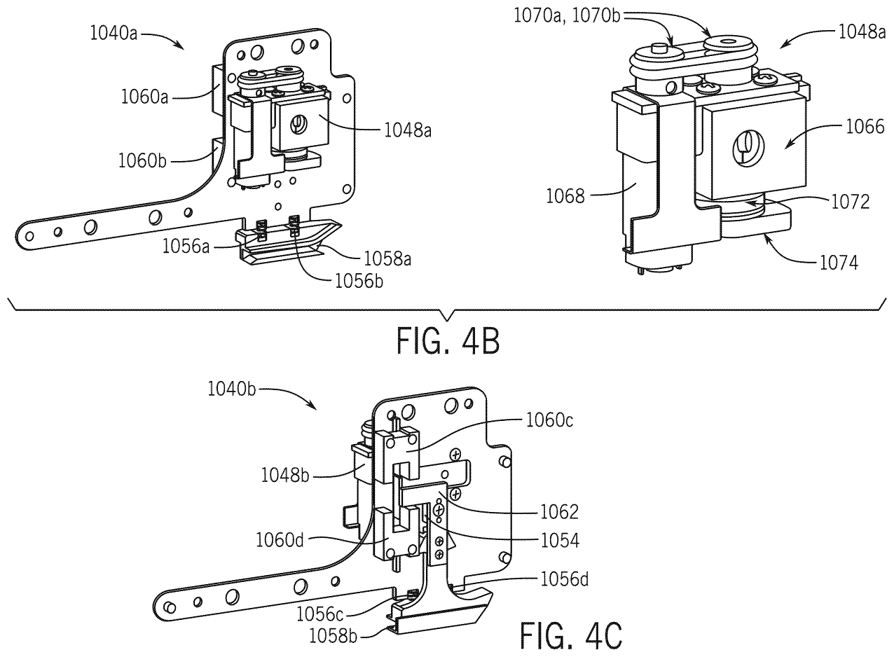

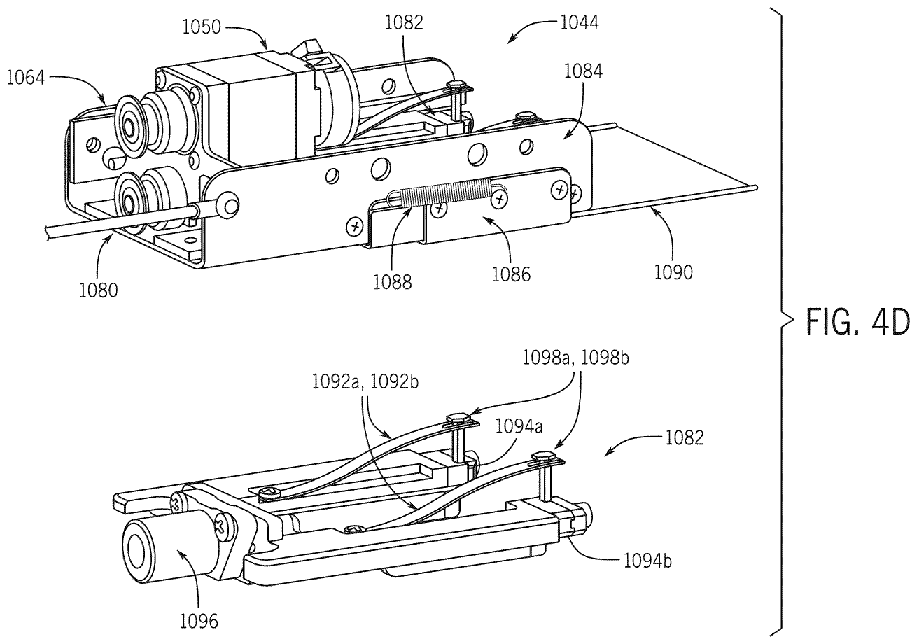

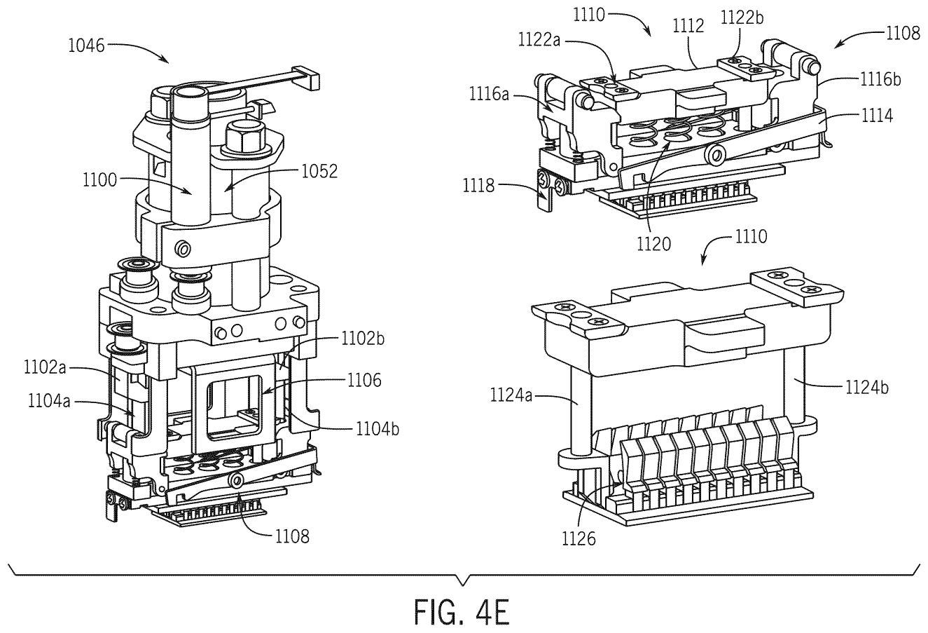

Referring now to FIGS. 4A-4E, various internal assemblies corresponding to handheld device 1000 are shown, according to some configurations. FIG. 4A shows the drive system 1034 that can include a left frame assembly 1040a, a right frame assembly 1040b, a horizontal component assembly 1044, or a vertical component assembly 1046. Each of the left and right frame assemblies 1040a, 1040b can include a corresponding flipper assembly (e.g., left flipper assembly 1048a, right flipper assembly 1048b). In some configurations, the horizontal component assembly 1044 can include a horizontal motor 1050. Further, the vertical component assembly 1046 can include a solenoid 1052.

Still referring to FIGS. 4A-4E, and in particular FIGS. 4B-4C, further exemplary details of the left and right frame assemblies 1040a, 1040b are shown, according to some configurations. In some configurations, the left frame assembly 1040a and the right frame assembly 1040b can be the same or substantially similar (e.g., symmetrical). As shown, the left frame assembly 1040a can include a left flipper assembly 1048a affixed to a first side of a left frame. Additionally, the left frame assembly 1040a can include flag sensors 1060a, 1060b, affixed to a second side of the left frame. The flag sensors 1060a, 1060b can communicate with a force sensing linear slide 1054. In some configurations, the left frame assembly 1040a can include force sensing springs 1056a, 1056b, which can contact a tissue interface 1058a. The tissue interface 1058a can be positioned on a third side of the left frame. In some configurations, the left frame assembly 1040a can attach to a portion of the vertical component assembly 1046 via screws and alignment pins, or other attachment systems.

In some configurations, the right frame assembly 1040b can include flag sensors 1060c, 1060d, affixed to a first side of a right frame. The flag sensors 1060c, 1060c can communicate with a force sensing linear slide 1054. Additionally, as shown, the right frame assembly 1040b can include a right flipper assembly 1048b affixed to a second side of the right frame. In some configurations, the right frame assembly 1040b can include force sensing springs 1056c, 1056d, which can contact a tissue interface 1058b. The tissue interface 1058b can be positioned on a third side of the right frame. In some configurations, the right frame assembly 1040b can attach to a portion of the vertical component assembly 1046 via screws and alignment pins.

The flipper assemblies 1048a, 1048b can include a flipper mounting block 1066, which can support a flipper motor 1068. In some configurations, the flipper mounting block 1066 can be constructed from a dielectric material. The flipper motor 1068 can be connected to (and control) flipper driver pulleys 1070a, 1070b. A bearing (e.g., a thrust bearing) 1072 can support an axial load exerted by the flipper motor 1068 on a flipper 1074. The flipper 1074 can rotate in accordance with motor actuation, and the flipper driver pulleys 1070a, 1070b can prevent any downward movement of the flipper 1074 during operation of the handheld device 1000. In some configurations, the flipper 1074 can include two connected components, such as two brass components that are brazed together. The primary function of the flipper 1074 can be to hold a needle top plate 1112 of FIG. 4E in place when loading needle retract springs. The flipper 1074 can then move out of the way of the solenoid 1052 during the remainder of normal operation. In some configurations, the flipper mounting block 1066 can act as a guide for solenoid plunger bars 1106 of FIG. 4E (e.g., to keep proper alignment).

Still referring to FIGS. 4A-4E, and in particular FIG. 4D, further exemplary details of the horizontal component assembly 1044 are shown, according to some configurations. The horizontal component assembly can include sensors, actuators, or guides for positioning a horizontal carriage assembly 1082 and, thereby, the hammers 1098a, 1098b used to drive microneedles into the tissue (as will be described below). In some configurations, a horizontal flag sensor 1064 can be used to position the horizontal component assembly 1082. As shown, the horizontal component assembly 1044 can include the horizontal carriage assembly 1082 that can be configured to mount the horizontal motor 1050. In some configurations, a horizontal chassis 1084 can support the horizontal carriage assembly 1082. Additionally, the right frame assembly 1040b and the left frame assembly 1040a can be affixed to opposing sides of the horizontal chassis 1084, for example, using rivets. An earth-ground connection 1080 can be attached to the horizontal chassis 1084, according to some configurations.

In some configurations, the horizontal component assembly 1044 can further include a retractable slide door 1090. The slide door 1090 can extend across the loading aperture 1006 when the cartridge 2002 has not been inserted into the engagement slot 1002. Accordingly, a user can be prevented from placing anything into the handheld device 1000 during the absence of the cartridge 2002. The sliding door 1090 can be secured to a sliding door mount 1086, which can be affixed to the horizontal chassis 1084. Additionally, a sliding door spring 1088 can be secured to the sliding door mount 1086, and biased such that the slide door 1090 remains in a “closed” position (i.e., extended across the loading aperture 1006) when the handheld device 1000 is not in-use or powered off.

As shown, the horizontal carriage assembly 1082 can include hammers 1098a, 1098b, corresponding hammer return springs 1092a, 1092b, and corresponding hammer guides 1094a, 1094b, according to some configurations. Generally, the horizontal carriage assembly 1082 can be configured to position and guide the hammers 1098a, 1098b to drive the microneedles into the tissue. In some configurations, the hammer guides 1094a, 1094b can be made of bronze, which can help to maintain bearing surfaces throughout many harvesting and scattering cycles. Additionally, in some configurations, the hammers 1098a, 1098b can be hardened 17-4 stainless steel, which can provide superior wear characteristics while maintaining anti-corrosion properties. The horizontal carriage assembly 1082 can further include a horizontal leadscrew drive nut 1096, which can include steps on the ends that can interface with ball bearings. Additionally, the horizontal leadscrew nut 1096 can be a Teflon-coated lead screw, and an Acetal drive nut designed to reduce friction. The horizontal leadscrew nut 1096 can provide a pitch adequate for positional resolution and linear force. The horizontal carriage assembly 1082 can additionally use stalling to sense whether or not a cartridge is loaded, or if there is a device jam.

Still referring to FIGS. 4A-4E, and in particular FIG. 4E, further exemplary details of the vertical component assembly 1046 are shown, according to some configurations. As shown, the vertical component assembly 1046 can include the solenoid 1052 and corresponding solenoid plunger bars 1106. Additionally, the vertical component assembly 1046 can include a vertical motor 1100, and associated unlock cams 1102a, 1102b and vertical leadscrews 1104a, 1104b. In some configurations, the vertical position of the vertical component assembly 1046 can be controlled by traveling up and down on the vertical leadscrews 1104a, 1104b (e.g., using the vertical motor 1100). As will be described, vertical positioning can move each of the microneedles corresponding to the cartridge 2002. In general, the vertical component assembly 1046 can be configured to interface with and manipulate the cartridge 2002 and its associated components during harvesting or scattering of tissue. In some configurations, the vertical motor 1100 can be sized to fit within the vertical component assembly 1046 while still providing the torque and speeds necessary for manipulating the microneedle positions.

In some configurations, the solenoid 1052 can deliver an operating force to the hammers 1098a, 1098b during harvesting or scattering. The solenoid 1052 can be activated by a half wave of AC current, as one non-limiting example. The force delivered by the solenoid 1052 can increase sharply, towards the end of its stroke. In some configurations, the mass of the solenoid plunger bars 1106a, 1106b can be selected based on the energy needed to drive the microneedles into the tissue. In some configurations, a stop (e.g., a brass stop) can be integrated into the solenoid 1052, which can enable extension control of the solenoid plunger bars 1106a, 1106b.

In some configurations, the vertical component assembly 1046 can include a vertical carriage assembly 1108. The vertical carriage assembly 1108 can be configured to support the solenoid 1052 and related components. As shown, the vertical carriage assembly 1108 can include a needle retract slide 1110 with a top plate 1112. In some configurations, opposite ends of the vertical carriage assembly 1108 can include needle retract slide-latches 1116a, 1116b with corresponding latch plates 1122a, 1122b. The latch plates 1122a, 1122b can define a maximum position of the needle retract slide 1110. Additionally, needle retract springs 1120 can be integrated into the vertical carriage assembly 1108, such that efficient retraction of the microneedles can be achieved. The needle retract slide-latches 1116a, 1116b can be used to lock down the needle retract slide 1110 in preparation for harvesting. The vertical carriage assembly 1108 can move both the needles and pins (e.g., pins within the microneedles) at the same time.

In some configurations, the vertical carriage assembly 1108 can include a cartridge latch 1114, which can be configured to secure the cartridge 2002 upon insertion into the loading aperture 1006. Additionally, a vertical flag 1118 can be affixed to the exterior of the vertical carriage assembly 1108, according to some configurations. As shown, the needle retract slide 1110 can further include guideposts 1124a, 1124b, which can be configured to guide the needle retract slide 1110 during vertical movement. In some configurations, the needle retract slide 1110 can include lockdown latches 1126, which can be in contact with the guideposts 1124a, 1124b, and configured to engage and disengage the microneedles during operating of the handheld device 1000. The needle retract slide 1110 can be a spring loaded subassembly that serves at least two purposes. First, the slide 1110 can retract the needles. Second, the slide 1110 can lock needle modules down (after being driven into the tissue). In some configurations, the needle retract slide 1110 is only capable of retracting the needles, and cannot move the needles forward. Additionally, in some configurations, the lockdown latches 1126 can be only functional after the skin grafting system 3000 has gone through initialization. Further detail regarding the operation of the skin grafting system 3000 is provided below.

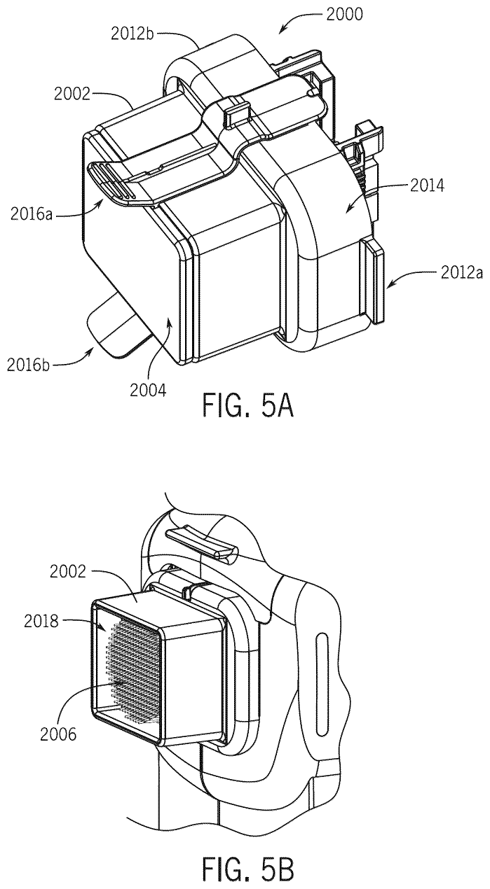

Referring now to FIGS. 5A-5B, the cartridge 2002 and a cartridge assembly 2000 are shown, according to some configurations. As shown, the cartridge assembly 2000 can include the cartridge 2002, and a cartridge cover 2004 that can be removably affixed to a microneedle chamber 2018. The microneedle chamber 2018 can enclose a plurality of microneedles 2006. In some configurations, the microneedles 2006 can be arranged as an array within the microneedle chamber 2018. As shown by FIG. 5A, the combination of the cartridge cover 2004 and the microneedle chamber 2018 can form a sealed enclosure for the microneedles 2006. The cartridge cover 2004 can include release levers 2016a, 2016b, which can be simultaneously depressed by a user to remove the cartridge cover 2004 from the cartridge 2002.

In some configurations, the cartridge 2002 can include a tissue stabilizer 2014, which can be configured to stabilize tissue during harvesting or scattering. Advantageously, the tissue stabilizer 2014 can be wider than the microneedle chamber 2018, allowing for a greater distribution of force during use of the skin grafting system 3000 on tissue. As shown, the tissue stabilizer 2014 can further include loading tabs 2012a, 2012b that extend outwardly. In some configurations, the loading tabs 2012a, 2012b can slide into contact with the engagement slot 1002 during loading of the cartridge assembly 2000 into the loading aperture 1006.

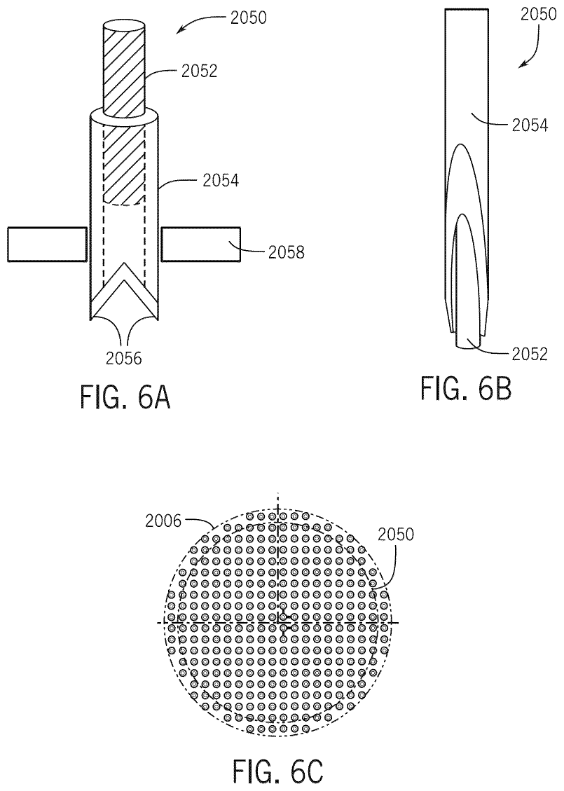

Referring now to FIGS. 6A-6C, a microneedle 2050 and a microneedle array 2006 are shown, according to configurations of the present disclosure. The microneedle 2050 can facilitate harvesting of tissue from a donor site. In some configurations, the microneedle 2050 can include a hollow tube 2054 that can include a plurality of points 2056 at the distal end thereof. In some non-limiting examples, needle systems such as described in U.S. Pat. Nos. 9,060,803; 9,827,006; 9,895,162; and US Patent Application Publication Nos. 2015/0216545; 2016/0015416; 2018/0036029; 2018/0140316 or combinations or components thereof can be used.

In some configurations of the present disclosure, the hollow tube 2054 can be provided with two points 2056, and the points 2056 can be sufficiently angled for penetrating and cutting the biological tissue to remove small micrografts therefrom. Such a hollow tube 2054 can be provided with two points 2056, and can use a force approximately twice that associated with a single-point needle of similar diameter to penetrate tissue.

In some configurations, the hollow tube 2054 can be slidably attached to a substrate 2058, such that the hollow tube 2054 can pass through a hole provided in the substrate 2058, as shown in FIG. 6A. The position of the hollow tube 2054 relative to the substrate 2058 can be controlled by translating the hollow tube 2054 relative to the substrate 2058, e.g., substantially along the longitudinal axis of the hollow tube 2054. In this manner, the distance that the distal end of the hollow tube 2054 protrudes past the lower surface of the substrate 2058 can be controllably varied.

The microneedle 2050 can further include a pin 2052 provided in the central lumen or opening of the hollow tube 2054. The diameter of the pin 2052 can be substantially the same as the inner diameter of the hollow tube 2054 or slightly smaller, such that the pin 2052 can be translated along the axis of the hollow tube 2054 while filling or occluding most or all of the inner lumen of the hollow tube 2054. The pin 2052 can be formed of a low-friction material, or coated with a low-friction material such as, e.g., Teflon® or the like, to facilitate motion of the pin 2052 within the hollow tube 2054 or inhibit accumulation or sticking of biological material to the pin 2052. The distal end of the pin 2052 can be substantially flat to facilitate displacement of a tissue micrograft within the hollow tube 2054 when the pin 2052 is translated.

The pin 2052 can be translated relative to the hollow tube 2054, e.g., substantially along the longitudinal axis of the hollow tube 2054. In this manner, the position of the distal end of the hollow tube 2054 relative to that of the distal end of the pin 2052 can be controllably varied. For example, the location of the distal ends of both the hollow tube 2054 and the pin 2052 relative to that of the lower surface of the substrate 2058 can be controllably and independently selected and varied.

FIG. 6B shows one configuration of the present disclosure, in which the pin 2052 can be positioned relative to the hollow tube 2054 such that their distal ends are substantially aligned. Portions of the pin 2052 or hollow tube 2054 can optionally be provided with a coating or surface treatment to reduce friction between them or between either component or biological tissue.

As described herein, a plurality of microneedles (e.g., microneedle 2050) can form a microneedle array 2006. FIG. 6C shows a top view of an exemplary microneedle array 2006, according to configurations of the present disclosure. In some configurations, the microneedle array 2006 can be substantially circular. The microneedle array 2006 can be formed by assembling a plurality of rows of needles, either horizontal or vertical rows. This design can be modular, and the configuration can take on any shape or size using various size rows as modules. In some configurations, all of the microneedles can be actuated, e.g., inserted into the tissue, simultaneously. In other configurations, groups or sections can be actuated sequentially. For example, the microneedle array 2006 can be divided into quadrants and each quadrant can be sequentially actuated. Sequentially can refer to actuating each row in a linear order, (e.g., row1, row2, row3), or non-linear (e.g. row1, row10, row3). Or, each row of microneedles can be separately and sequentially actuated. Additionally, each single microneedle can be separately and sequentially actuated. In some configurations, one row can be actuated at a time, e.g., 20 rows can be individually actuated in sequence, while in other configurations, two, three, four or more rows can be actuated at a time. An advantage to sequentially actuating segments of the microneedle array 2006 is that insertion of a segment can require less force on the donor site than insertion of the entire microneedle array 2006. In some configurations, the microneedle array 2006 can be driven using a solenoid (e.g., solenoid 1052). Multiple actuations using the solenoid can sequence the insertion row by row.

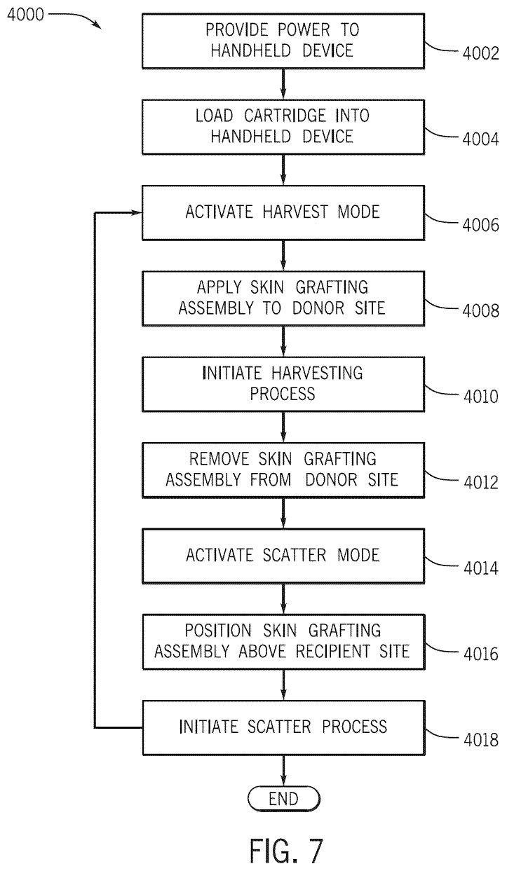

Referring now to FIG. 7, some non-limiting examples of steps of a process 4000 for harvesting and scattering tissue is shown, according to configurations of the present disclosure. In some configurations, the process 4000 can be implemented using the skin grafting system 3000, as described above. As shown, the process 4000 includes providing power to the handheld device (process block 4002). In some configurations, the handheld device can be the same or similar to handheld device 1000. The process 4000 is shown to further include loading a cartridge into the handheld device (process block 4004). In some configurations, the cartridge can be the same or similar to cartridge 2002, or cartridge assembly 2000. Further, the process 4000 is shown to include activating a harvest mode (process block 4006). This activation can be initiated via user interface 1008, according to some configurations, such as will be described. The process 4000 is shown to include applying a skin grafting system (e.g., skin grafting system 3000) to a donor site (process block 4008). The donor site can correspond to a healthy area of tissue on a patient. Next, the process 4000 is shown to include initiating a harvesting process (process block 4010). In some configurations, this initiation can occur via the above-described trigger 1014. The process 4000 is shown to further include removing the skin grafting system from the donor site (process block 4012). Next, the process 4000 is shown to include activating a scatter mode (process block 4014). In some configurations, this activation can occur via user interface 1008, such as will be described. The process 4000 is shown to further include positioning the skin grafting system above a recipient site (process block 4016). In some configurations, the recipient site can correspond to a damaged area of tissue on the patient. Next, the process 4000 is shown to include initiating a scatter process (process block 4018). In some configurations, this initiation can occur via actuation of the above-described trigger 1014. As shown, the process 4000 can end after the scatter process (process block 4018), or can return to process block 4006 to reactivate the harvest mode. In some configurations, a single cartridge (e.g., cartridge 2002) can be used multiple times on the same patient. Advantageously, if the recipient site is relatively large, multiple harvests and scatters can occur using a single cartridge. Accordingly, the process 4000 can continue with process blocks 4006 through 4018 until a user is ready to dispose of the cartridge.

According to configurations of the present disclosure, the harvest process and scatter process can be performed using skin grafting system 3000. A non-limiting description of the internal functions of the handheld device 1000 and cartridge 2002 are accordingly disclosed herein.

### User Interface

Referring to FIG. 2B, as one non-limiting example, an example of using the user interface 1008 to control the above-described process is provided. Upon providing power to the handheld device, the stand-by input 1018 can flash white when the handheld device 1000 first powers on (e.g., for ~8 seconds at initial start-up). This can inform the user that the handheld device 1000 is performing a start-up self-test or other operation. As another non-limiting example, the stand-by input 1018 can produce steady green illumination when the handheld device 1000 is on and ready for subsequent use. In some configurations, pressing the stand-by input 1018 for a pre-determined amount of time (e.g., 3 seconds, 5 seconds, or the like) can cause the handheld device 1000 to enter a stand-by mode. Continuing with the non-limiting example, the stand-by input 1018 can stop producing light when the handheld device 1000 is in stand-by mode. Other light colors, patterns, and timing can be implemented, according to various configurations and preferences.

As another non-limiting example, the indicator light 1020 can produce steady white light when the handheld device 1000 is in harvest mode but sufficient pressure against a donor site has not been achieved, such as will be described during a skin grafting process. Further, the indicator light 1020 can produce steady green light when the handheld device 1000 is in harvest mode and sufficient pressure against the donor site has been achieved (and the trigger 1014 is disengaged). The indicator light 1020 can produce flashing green light when the handheld device 1000 is in the process of harvesting. If pressure drops below a threshold value during the harvesting process, the indicator light 1020 can produce flashing white light. Further, the indicator light 1020 can produce flashing white light when the handheld device 1000 is experiencing a fault condition.

In another non-limiting example, the scatter input 1022 can produce steady white light when the harvest process is complete. In some configurations, a subsequent press of the scatter input 1022 can cause the handheld device 1000 to enter a scatter mode. The scatter input 1022 can produce steady green light when the handheld device 1000 is in scatter mode. Similar to the indicator light 1020, the scatter input 1022 can produce flashing white light when the handheld device 1000 is experiencing a fault condition. In some configurations, the scatter input 1022 can produce flashing white light during the harvesting process, which can indicate that extraction recovery is needed. A subsequent press of the scatter input 1022 can activate an extraction recovery process. Once the extraction recovery process is complete, the scatter input 1022 can stop producing light. A detailed description of the extraction recovery process is provided below.

In some configurations, similar to the indicator light 1020, the indicator light 1016 can produce a solid green light when the handheld device 1000 is in the harvest mode and sufficient pressure against the donor site has been achieved (and the trigger 1014 is disengaged). Additionally, the indicator light 1020 can produce flashing green light during the harvesting process, according to some configurations.

### Skin Grafting System Operating Positions

In some configurations, a plurality of operating positions corresponding to the skin grafting system 3000 can be defined. Notably, the skin grafting system 3000 can operate using additional operating positions not explicitly defined.

Some configurations of the present disclosure include a horizontal carriage home position, where the horizontal carriage assembly 1082 can be in a position that occludes the horizontal flag sensor 1064. This position can be a “safe” position that keeps the carriage away from other moving parts. From a user's perspective, it can appear the horizontal carriage assembly 1082 is retracted back inside the handheld device 1000.

Some configurations of the present disclosure include a vertical carriage home/harvest position, corresponding to a calibrated position where the vertical carriage assembly 1108 can be aligned the corresponding components for loading or for harvesting. This position can be below the vertical flag sensor occlusion point. From a user's perspective, it can appear that the vertical carriage assembly 1108 is closest to the engagement slot 1002 of the handheld device 1000.

Some configurations of the present disclosure include a vertical carriage unlock/scatter position corresponding to a calibrated position where the vertical carriage assembly 1108 has unlocked the needle retract slide 1110 by pushing the needle retract slide latches 1116a, 1116b over their respective unlock cams 1102a, 1102b. This can be the highest position the vertical carriage assembly 1108 will travel to. From a user's perspective, it can appear that the vertical carriage assembly 1108 is up inside the handheld device 1000.

Some configurations of the present disclosure include a “flipper in” position and a “flipper out” position. Each flipper 1074 can have two defined positions that the handheld device 1000 detects via flag sensors that can provide positive feedback that each position has been reached. The “flipper in,” or retracted, position can correspond to when the flipper 1074 is safely away from moving parts. The “flipper out,” or extended, position can correspond to when the flipper 1074 is blocking the top plate 1112. The “flipper out” position can be used for initialization, when the needle retract slide 1110 (and therefore the cartridge 2002) is locked.

Some configurations of the present disclosure include a vertical carriage lock position, corresponding to a calibrated position where the vertical carriage assembly 1108 can move to (with the flippers 1074 extended out) to compress the needle retract springs 1120 and to lock the needle retract slide latches 1116. This “locking” is what can allow the needles to later be retracted, while also locking the cartridge 2002 inside the handheld device 1000.

Some configurations of the present disclosure include a vertical carriage lock relax position, which can be a position that is offset from a calibrated lock position, where a properly locked needle retract slide top plate 1112 will no longer be putting pressure on the flippers 1074, and therefore the flippers 1074 can be safe to retract in. Conversely, if the needle retract slide top plate 1112 is not properly locked, this position can be designed to maintain enough pressure on the flippers 1074 so that they will not retract in. This position can enable the handheld device 1000 to positively sense a proper locking of the needle retract slide 1110.

Some configurations of the present disclosure include a vertical carriage extract position, which can be a position that is offset from a calibrated unlock position, where the needle retract slide 1110 will not be unlocked and the extended needles can be behind the tissue stabilizer 2014. After harvest, this position is where the vertical carriage assembly 1108 can go to extract the needles (containing the tissue grafts) prior to scattering. Advantageously, tissue grafts may not be exposed in this position, as the needles remain extended.

Some configurations of the present disclosure include a harvest recovery mode, which can occur during the harvest process. The harvest recovery mode can include attempting to continue deploying the needle modules into the tissue. Additionally, the harvest recovery mode can be automatic and fully controlled by on-board software (i.e., no user interaction required).

Some configurations of the present disclosure include an extraction recovery mode, which can occur after the needles have been deployed (and the handheld device 1000 is attempting to return the horizontal carriage home position). In some configurations, it can be possible for the horizontal carriage assembly 1082 to get stuck due to increased friction from the needle modules. If this occurs, the handheld device 1000 can blink the scatter light (on the scatter input 1022) white, indicating that an extraction recovery is needed. The user may then relieve the downward force on the tissue, and press the scatter input 1022, which will allow the handheld device 1000 to continue with extracting the needles from the tissue.

### Skin Grafting Assembly Vertical Operation

Various components corresponding to the handheld device 1000 and cartridge 2002 can have a predefined operation based on the current mode of the handheld device 1000 (e.g., initialization, harvest mode, scatter mode, etc.), according to some configurations.

In some configurations, the vertical component assembly 1046 can have a predefined “loading” configuration that corresponds to loading of the cartridge 2002 into the handheld device 1000. During loading, for example, the solenoid plunger bars 1106a, 1106b, the each flipper 1074 can be retracted (flipper in), and the needle retract slide 1110 can be retracted (the needles retracted). The vertical carriage assembly 1108 can be set to the home position (as described above).

In some configurations, the vertical component assembly 1046 can have a predefined “initialization” configuration. During initialization, for example, each flipper 1074 can be extended (flipper out), and the needle retract slide 1110 can be locked with the needle retract springs 1120 loaded (the needles remain retracted). The vertical carriage assembly 1108 can be set to the lock position (see above). With each flipper 1074 extended, the vertical carriage assembly 1108 can move up to the lock position. The extended flippers 1074 can hold the needle retract slide 1110 in place. When the vertical carriage assembly 1108 reaches the lock position, the needle retract slide latches 1116 can lock the top plate 1112 in place with the needle retract springs 1120 loaded. In some configurations, this does not move the needles from their retracted state.

In some configurations, the vertical component assembly 1046 can have a predefined “initialized” configuration, which can correspond to the skin grafting system 3000 being ready to harvest. During the initialized configuration, for example, each flipper 1074 can be retracted (flipper in), and the needle retract slide 1110 can be locked with the needle retract springs 1120 loaded. In some configurations, this does not move the needles from their retracted state. The vertical carriage assembly 1108 can move back down to the home position, according to some configurations.

In some configurations, the vertical component assembly 1046 can have a predefined “harvest” configuration corresponding to an applied user force. During the harvest configuration, for example, the needle retract slide 1110 can remain locked with the needle retract springs 1120 loaded and the needles retracted. The vertical carriage assembly 1108 can remain in the home position, according to some configurations. When the user positions the skin grafting system 3000 at the donor site and applies downward force, the tissue stabilizer 2014 can move a small amount, causing proper alignment for harvest. In some configurations, the indicator light 1016 can illuminate green, to provide a visual confirmation of force to the user.

In some configurations, the vertical component assembly 1046 can have a predefined “harvest” configuration corresponding to needle deployment. During this harvest configuration, for example, the solenoid plunger bars 1106a, 1106b can advance, the needle retract slide 1110 can remain locked with the needle retract springs 1120 loaded. Notably, the needles (e.g., from microneedle array 2006) can be deployed into the tissue. The vertical carriage assembly 1108 can remain at the home position, and a user force can still be applied via the handheld device 1000, according to some configurations. When the user pulls the trigger 1014, the skin grafting assembly 3000 can begin the harvest sequence. Accordingly, the skin graft assembly 3000 can advance each microneedle array row of needles into the tissue by hitting the hammers 1098a, 1098b with the solenoid plunger bars 1106a, 1106b.

In some configurations, the vertical component assembly 1046 can have a predefined “extraction” configuration. During the extraction configuration, for example, the solenoid plunger bars 1106a, 1106b can be retracted, the needle retract slide 1110 can remain locked with the needle retract springs 1120 loaded. The needles (e.g., from microneedle array 2006) can remain deployed into the tissue. The vertical carriage assembly 1108 can move to the extraction position (described above), and the user force can be removed from the handheld device 1000. In some configurations, after the harvest is complete, the skin grafting system 3000 can extract the needles by lifting all of needles within the microneedle array 2006 at once. The needles can be lifted up to the extraction position. In some configurations, the needles can remain advanced relative to the pins (e.g., pin 2052) and the tissue stabilizer 2014 can remain stationary when the needles are retracted.

In some configurations, the vertical component assembly 1046 can have a predefined “scatter” configuration. During the scatter configuration, for example, the needle retract slide 1110 can be in a retracted position, with the needles similarly retracted. In some configurations, the vertical carriage assembly 1108 can move from the locked position. When the user activates the scatter sequence, the skin grafting system 3000 can move the vertical carriage assembly 1108 from the locked position, which can release the loaded needle retract springs 1120, and the needle retract slide 1110. Accordingly, this movement can retract the needles relative to the pins (e.g., pin 2052), thus exposing the grafts and positioning the components for a scatter sequence.

In some configurations, the vertical component assembly 1046 can have a “scatter” configuration corresponding to an advanced needle position. During this scatter configuration, for example, the solenoid plunger bars 1106a, 1106b can advance, and the needle retract slide 1110 can advance (similarly, the needles can advance). According to some configurations, the solenoid plunger bars 1106a, 1106b can advance, first hitting the top plate 1112, and then hitting the needle modules (e.g., within microneedle array 2006). This can push the top plate 1112 ahead of needle carriers, thus preventing damage to the carriers. The advancing of the needles, followed by the rapid retraction of those needles (by the unlocked top plate 1112) can disperse the grafts into the recipient site.

### Power On Self-Test

In some configurations, the handheld device 1000 can perform a self-test upon start-up (e.g., when the handheld device 1000 is first powered on). In some configurations, the self-test can occur when the handheld device 1000 is plugged in to receive power, and the stand-by input 1018 (which can be illuminated white), is pressed and released. The stand-by input 1018 can flash green throughout the duration of the self-test, according to some configurations. Next, the horizontal carriage assembly 1082 can move a very small amount forward, such that the horizontal flag sensor 1064 is cleared. Subsequently, the horizontal carriage assembly 1082 can return to the home position.

During the self-test, the vertical carriage assembly 1108 can move a very small amount upwards, such that the vertical flag 1118 clears the sensor. Subsequently, the vertical carriage assembly 1108 can return to the home position. In some configurations, the vertical carriage assembly 1108 can move up to the unlock position, where it can move the needle retract slide latches 1116, before returning to the home position. This can, for example, release the needle retract slide 1110, in the event that it is locked (e.g., cartridge 2002 is locked in).

In some configurations, the horizontal carriage assembly 1082 can move to a predetermined position (e.g., approximately two-thirds of the way through its full range), which can verify that a cartridge (e.g., cartridge 2002) is not present. Subsequently, the horizontal carriage assembly 1082 can return to the home position.

During the self-test, the flippers 1074 can extend out and then retract back in. Further, in some configurations, some or all lights on handheld device 1000 can flash (e.g., indicator light 1016, 1020, scatter input 1022, etc.). Upon completion of the self-test, the stand-by input 1018 can lights up solid green, for example, which can indicate that the self-test was successful.

### Cartridge Loading and Initialization

In some configurations, the skin grafting system 3000 can have a predefined cartridge loading and initialization process. The user can open the loading door 1004, then slide the cartridge assembly 2000 (i.e., including the cartridge cover 2004) into the engagement slot 1002. The cartridge latch 1114 can lock onto the cartridge 2002. The user can then remove the cartridge cover 2004 and close the loading door 1004, which can activate the internal loading door switch.

The initialization process can further include moving the horizontal carriage assembly 1082 from the home position, such that it can detect the cartridge presence by stalling on the first cartridge segment. Subsequently, the horizontal carriage assembly 1082 can return to the home position. Additionally, the vertical carriage assembly 1108 can move a small amount, such that the vertical flag 1118 clears the sensor, and then the vertical carriage assembly 1108 can return to the home position.

In some configurations, the flippers 1074 can extend out above the top plate 1112. The vertical carriage assembly 1108 can move to the lock position. While moving to the lock position, the flippers 1074 can hold the top plate 1112 in place while the needle retract slide latches 1116 move out, and eventually lock over the top plate 1112. Accordingly, the needle retract springs 1120 can be held in a compressed state. While this is happening, for example, the lockdown latches 1126 can spring out under the needle segments (e.g., within the microneedle array 2006), in preparation for locking the needle segments down during the harvest sequence. In some configurations, the vertical carriage assembly can then move a small amount down, thus moving into the lock relax position (described above). Additionally, the flippers 1074 can retract back in.

The initialization process can further include returning the vertical carriage assembly 1108 to the home/harvest position. The horizontal carriage assembly 1082 can engage with the first needle segment (within microneedle array 2006) by stalling against the segment and subsequently backing off by a small, predetermined distance. The handheld device 1000 can then calculate the position of each needle segment. Upon completion of the initialization process, the indicator light 1020 can illuminate white to indicate that the handheld device 1000 is ready for the harvest sequence.

### Methods of Harvest and Extraction

In some configurations, a user can harvest and extract tissue columns using a harvesting process. The user can position the handheld device 1000 at the donor site, with the tissue stabilizer 2014 pressed against the skin. The user can use two hands to apply force against the skin via the handheld device 1000. The tissue stabilizer interface components can move upward, compressing the force sensing springs 1056 until the force sensing flag 1062 occludes the flag sensor. In some configurations, the indicator lights 1016, 1020 can illuminate green, thus indicating that the trigger 1014 is active.

Once the trigger 1014 is active, the user can pull the trigger 1014 (while maintaining the force on the skin) and the handheld device 1000 can begin the harvest sequence. In some configurations, the indicator lights 1016, 1020 can blink green throughout the duration of the harvest and the extraction. The force sensing flag 1062 can be monitored throughout the harvest (between solenoid activations) to ensure that sufficient force is maintained. The solenoid 1052 can rapidly advance the solenoid plunger bars 1106a, 1106b, which can advance the two hammers 1098a, 1098b, and insert the first needle module into the tissue. The needle module travels past the needle module lockdown latches as it is inserted. Subsequently, the solenoid 1052 and hammers 1098a, 1098b can retract, and the needle segment can remain locked down in the tissue.

In some configurations, the horizontal carriage assembly 1082 can advance to the calculated position of the next needle segment. The solenoid 1052 can rapidly advance the solenoid plunger bars 1106a, 1106b, which can advance the two hammers 1098a, 1098b, and insert the next needle module into the tissue. The needle module can travel past the lockdown latches 1126 as it is inserted. The lockdown latches 1126 can spring back out, and the solenoid 1052 and hammers 1098a, 1098b can retract. This insertion process can repeat until all needle segments have been inserted into the tissue.

After completing the insertion of all segments, the horizontal carriage assembly 1082 can return to the home position, according to some configurations. The vertical carriage assembly 1108 can move up to the extraction position, extracting the needles from the tissue, and positioning the needles safely up inside the tissue stabilizer 2014. The indicator lights 1016, 1020 can stop blinking green and turn off. Additionally, the scatter input 1022 can be illuminated white, indicating that the handheld device 1000 is ready to proceed with the scattering process. Upon completion of the harvesting process, the user can remove the force on the tissue, and lift the handheld device 1000 away.

### Methods of Scatter

In some configurations, a user can scatter the tissue columns after the harvesting process. Once the user has removed the handheld device 1000 from the donor site (with the tissue columns harvested), the needles can be safely up inside of the tissue stabilizer 2014. With the recipient site ready for the tissue grafts, the user can activate the scatter mode by pressing the scatter input 1022. In some configurations, the scatter input 1022 can change from being illuminated white to green.

In some configurations, the user can position the tissue stabilizer 2014 directly above the recipient site. The user can then pull the trigger 1014 and the vertical carriage assembly 1108 can move out of the lock position, which can release the needle retract slide 1110 and retract the needles behind the pins (e.g., pins 2052). The handheld device 1000 can rapidly advance the solenoid plunger bars 1106a, 1106b which accordingly push both the needle retract slide 1110 and the needle modules. The needle retract slide 1110 can remain pushed ahead of the needle modules to prevent damage to the needles. Subsequently, the solenoid plunger bars 1106a, 1106b can be retract, which can cause the needle retract slide 1110 to retract (pulling the needle modules back with the needle retract slide 1110). The process of rapidly advancing the solenoid plunger bars 1106a, 1106b can be repeated several times, which can ensure that as many grafts as possible have been deposited into the recipient site. In some configurations, six activations of the solenoid 1052 can occur. After the scatter process has completed, the vertical carriage assembly 1108 can return to the home position, with the needle retract slide 1110 unlocked.

### Cartridge Removal

In some configurations, once the user has completed the harvest and scatter processes, the user can open the loading door 1004, depress the cartridge latch 1114, and slide the cartridge 2002 out. In some configurations, if the user wants to complete another harvest with the same cartridge 2002, the user can open and close the loading door 1004 (i.e., without removing the cartridge 2002). This can begin another initialization process via the handheld device 1000.

The general configuration of systems and methods illustrated in FIGS. 1-7 can be modified to provide extended, improved, or alternate ways of performing certain operations associated with the above-described skin grafting procedures. For example, any one or more of the above-described needle arrays (e.g., microneedle array 2006), actuation systems (e.g., drive system 1034), energy systems (e.g., power module 1028), feedback systems (e.g., user interface 1008), stabilizers (e.g., tissue stabilizer 2014), scattering assemblies (e.g., cartridge assembly 2000), form factors (e.g., housing 1036), and combinations thereof can be modified to provide additional or alternate advantages. Moreover, additional systems or elements not expressly illustrated in FIGS. 1-7 can be added to supplement the above-described skin drafting devices and procedures.

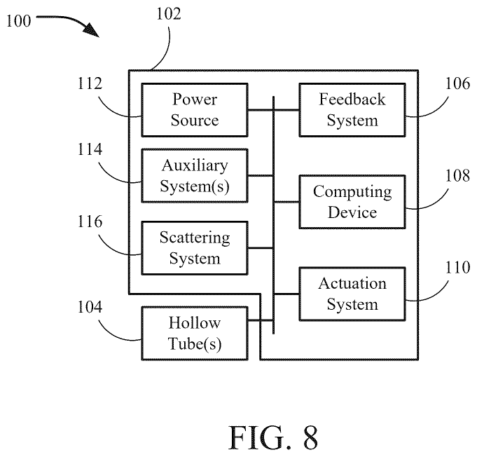

FIG. 8 shows a schematic illustration of a general configuration of a system 100, which can be used for skin grafting, and particularly autologous skin grafting (e.g., harvesting skin tissue and subsequently depositing the harvested skin tissue on a target site, which can be a wound site). Thus, the system 100 can be referred to as a skin grafting system 100. The system 100 pertains to other systems described herein including the skin grafting system 3000 shown in FIGS. 1-7. Accordingly, features, components, etc., of the system 100 are applicable to the system 3000 and vice versa. As shown in FIG. 8, the system 100 can include a housing 102 (e.g., the housing 1036 of the handheld device 1000, a housing in the form of a handheld device, etc.), a plurality of hollow tubes 104, which can be configured within a cartridge (e.g., such as the cartridge 2002), a feedback system 106 (e.g., such as the user interface 1008) that can be coupled to or otherwise integrated within the housing 102, a computing device 108 (e.g., the main controller 1032), an actuation system 110 (e.g., the drive system 1034), a power source 112 (e.g., the power module 1028) , one or more auxiliary systems 114, and a scattering system 116. Various examples of the modified or alternative components shown in FIG. 8 and others are here described.

As shown in FIG. 8, all of the components of the system 100 can be coupled to and can be integrated within the housing 102 to at least some extent. However, in other configurations, some or other components of the system 100 can be separate from (and not coupled to the housing 102). For example, the feedback system 106 can include a display that is in communication with the system 100, but the display can be separate from the housing 102 (e.g., a monitor, personal computer, a smartphone, etc.). As another example, the power source 112 can include a battery pack having and securing one or more batteries, which can be rechargeable. In this case, then, the battery pack can be separate from the housing 102, during, for example, charging of the battery pack.

FIG. 8 shows the hollow tube(s) 104, which can be implemented according to any of the applicable configurations herein. For example, the system 100 can include a cartridge that includes a plurality of hollow tubes. As another example, the hollow tube(s) 104, which can be a plurality of hollow tubes can be coupled to the housing 102. In this case, the entire system 100 can be disposed of after harvesting, scattering, or both. Each hollow tube 104 can be configured to harvest at least a portion of tissue from a tissue site when the respective hollow tube 104 is translated into the tissue site. For example, the actuation system 110 can translate each hollow tube 104 into the tissue site to harvest a portion of tissue therefrom. For the remaining portions of this application, the tissue site will be referred to as a skin tissue site. However, in other configurations, the system 100 can harvest, scatter, etc., tissue types other than skin. Once the skin tissue has been harvested from each hollow tube 104, the actuation system 110 (or other system) can translate the hollow tube(s) 104 out of the skin tissue site (e.g., raise the hollow tube(s) 104) out of the tissue site. At this point, each hollow tube 104 retains a respective portion of skin tissue (e.g., a micrograft) harvested from the skin tissue site. Subsequently, once the hollow tube(s) 104 are moved to a target site, such as, for example, a wound (e.g., a pressure wound that is having difficulty healing), a substrate, a collector (e.g., a funnel, bowl, etc.), the scattering system 116 can deposit the captured portions of tissue within the hollow tubes 104 onto the target site. For example, the scattering system 116 can force, eject, push, etc., each harvested tissue portion out of each hollow tube 104 and onto the target site. In some cases, the harvesting and scattering process can repeat a number of times or cycles until an amount of harvested skin tissue portions have been collected or dispersed adequately over a target site (e.g., a wound site).

In some configurations, the system 100 can include a plurality of reloadable springs. For example, each reloadable spring can correspond to a respective segment of hollow tubes (e.g., a row of hollow tubes). In other words, each reloadable spring can correspond for downward translation of a specific segment of hollow tubes (e.g., a row of hollow tubes). In some cases, one or more motors (e.g., a linear actuator) can load one or more of the reloadable springs. For example, a motor can load multiple (e.g., all) the reloadable springs at once, using, for example, the configuration described below. In some cases, the system 100 can selectively unload particular reloadable springs thereby driving corresponding segments of hollow tubes into the donor site. For example, this can be implemented pneumatically by using a plurality of pneumatic brakes, each of which prevents or otherwise locks a respective reloadable spring into a loaded configuration. These brakes can then be released electronically by causing a valve to open to release the brake and unload the particular reloadable spring, or alternatively, by causing rotation of a cam shaft that selectively opens a valve thereby releasing the brake to unload the reloadable spring.

In some cases, a plurality of hollow tubes can be actuated by a motor (e.g., a DC motor or a pneumatic motor) operably coupled to a camshaft that selectively causes rows or sections of the plurality of hollow tubes to enter the tissue (e.g., by causing a specific actuator to drive a particular segment into the tissue). As a specific example, and described in more detail below, a camshaft can selectively load each reloadable spring, by rotating in a first direction, and then correspondingly, can selectively release a given reloadable spring by further rotation of the cam shaft in the first direction. Regardless, unloading of the reloadable spring drives translation of the corresponding segment of hollow tubes into the donor site. In some cases, the loading force of the spring can be a predetermined force that can be substantially equal to the estimated or predicted peak force necessary to pierce the epidermis. In some cases, the spring loading force (e.g., the maximum spring loading force, for a given movement distance of the spring, such as the spring displacement) can be substantially greater than or equal to 1.5, 2, 25. lbs. per needle. For example, for a segment with 10 hollow tubes at 1.5 pounds per needle, the spring loading force would be at least 15 pounds.

In some configurations, the actuation system 110 can include one or more pneumatic actuators to drive one or more segments of hollow tubes into a donor site. For example, a pneumatic actuator can be used as opposed to a solenoid to quickly drive one or more segments segment of hollow tubes into the donor site (e.g., sequentially drive different segments of hollow tubes into a donor site). This can function in a similar manner as the solenoid (e.g., can be used in place of the vertical motor 1100). In other configurations, similarly to having multiple reloadable springs, a plurality of pneumatic actuators can be used, with each corresponding to a specific segment of hollow tubes (e.g., a row of hollow tubes). In a similar way, specific pneumatic actuators can be activated in a particular order to drive respective segments of hollow tubes into the donor site. Again, this can be implemented electronically (e.g., by selectively opening particular valves, via a computing device, such as a controller), or can be implemented by rotating a cam shaft to selectively open particular valves). In this case, when a valve is opened, pressure from a pressure source (e.g., a pump, a pump feeding an accumulator in series, etc.) can drive the particular pneumatic actuator thereby driving the corresponding segment of hollow tubes into the donor site.

In one example, the plurality of hollow tubes 104 can be actuated by an ultrasonic actuator or other piezo motor. The ultrasonic actuator can be a Langevin transducer which includes a piezoelectric element that can surround each hollow tube 104 and produces a force in the longitudinal direction of the hollow tube 104 in response to an applied voltage or current. Because ultrasonic actuators can generate an undesirable amount of heat, in such implementations the plurality of hollow tubes 104 can be in communication with a heat removal system such as a heatsink, a heat pipe, a phase change material, a fluid coolant, and the like. In some configurations, the ultrasonic transducer can be coupled to a portion of the skin grafting system. For example, the ultrasonic transducer can be coupled to a portion of the cartridge, a portion of the array of hollow tubes (e.g., needle array), a portion of the housing 102, etc. Regardless of the configuration, the ultrasonic transducer can vibrate one or more hollow tubes at ultrasonic frequencies as the one or more hollow tubes are translated downwardly, or otherwise moved into the donor site. In this way, the vibration of the hollow tubes at ultrasonic frequencies can reduce the insertion force required into the tissue, particularly when first puncturing the epidermis (e.g., which can correspond to the peak force required to insert a needle into skin tissue). However, as indicated above, undesirable heat could be transmitted to the donor site or harvested micrografts. Therefore, each hollow tube can include a respective heat sink, heat pipe, etc. to dissipate or otherwise remove heat generated by an ultrasonic transducer.

Certain actuation systems can be capable of operating with reduced power, as compared to implementations using the solenoid 1052. In such implementation, the power module 1028 can be removed or replaced. The power module 1028 as described above is configured to connect to an AC power supply (e.g., a mains voltage) in order to provide a current to the solenoid 1052 (e.g., to accommodate the typically large current draw required by the solenoid). However, other actuation systems such as a DC motor can be operable with a battery pack in addition to or instead of a mains power supply. In such implementations, a battery pack (e.g., including one or more batteries, such as lithium ion batteries, rechargeable batteries, etc.) can be included within the housing 102 or can be configured to removably attach to the housing. By using a battery pack, cordless operation can be achieved, which can be advantageous for better mobility and movement by a practitioner (e.g., doctor) during a harvesting, scattering, etc., procedure. In other words, the battery pack can prevent limited movement as restricted by a power cord, under some configurations.

In some configurations, the battery pack can be rechargeable. For example, the handheld device, which includes a rechargeable battery pack, can engage with a charging dock during downtime of the system 100 (e.g., when the device is not being used, such as in-between procedures, overnight, during a break, while preparing a patient, etc.). In this way, the handheld device can advantageously charge when not in use. In this regard, the handheld device can include two or more electrodes that can be electrically coupled to the charging dock when the charging dock is engaged with the handheld device. In some configurations, a cover can be placed over and can block exposure of electrodes while the handheld device is not being charged and not engaged with the charging dock. In this way, debris including patient bodily fluids (e.g., blood), are prevented from undesirably interacting with the electrodes.

In the example illustrated in FIGS. 1-2B, visual feedback is provided to a user via the stand-by input 1018, the indicator lights 1016 and 1020, and the scatter input 1022, which are elements of the user interface 1008. However, additional or alternative feedback elements can be provided. In general, the feedback elements (e.g., feedback system 106 of FIG. 8) can include any combination of visual feedback, auditory feedback, and haptic feedback. Moreover, while the example of FIGS. 1-2B provides feedback indicative of a mode of the handheld device, a process state of the handheld device, and so on, other types of feedback are within the scope of the feedback system 106. For example, the feedback system 106 can be configured to provide an indicator to the user that harvesting is complete (e.g., that the needles have had sufficient time to fully retract). Moreover, the interface elements are not limited to static elements (e.g., as shown in FIG. 2B), but can be or include dynamic elements.

As used herein, a “dynamic element” can be any visual, auditory, or haptic feedback that varies over time. In the example of visual feedback, a dynamic element can include a video display either via a display screen integrated with the skin grafting system or separate from and operably connected thereto (e.g., a monitor). The operable connection can be via a wired or wireless communication link to an external device having a display screen. In the example of auditory feedback, a dynamic element can include a speaker configured to provide dynamic audio output (e.g., spoken instructions) to the operator. Again, the speaker can be integrated with the skin grafting system or separate from and operably connected thereto, via either a wired or wireless communication link. In the example of haptic feedback, a dynamic element can include a variable vibration element (e.g., using a piezoelectric oscillator) to provide a haptic feedback that varies over time, and can be integrated or separate from the skin grafting system.

In one example, the cartridge can include one or more “funnel-type” attachments (e.g., which can be removably coupled to the housing 102) to direct the harvesting or scatter operations. In one particular example, this can be implemented as one or more clip-on attachments configured to interface with the cartridge or the housing 102. The attachments can be treated with a nonstick coating to reduce friction and prevent tissue portions from sticking to the attachments.

Moreover, when using the tissue stabilizer 2014 described above, “incomplete” harvests may occur at the center of the array, for example due to the skin being less taut than at the edges of the cartridge. Thus, the cartridge or the housing 102 more broadly can be equipped with additional or alternative stabilizers. In one example, a solid bar or bars can be placed down the center of the hollow tubes (e.g., the needle array) to provide additional tissue stabilization. In such examples, the needle array itself can be segmented so that the operation of the needle array is not impacted by the bar(s). As another example, the additional or alternative stabilizers can be or include a flat plate with holes sized to accommodate individual needle insertion, and can directly contact with the harvest site and increase tissue stability. The cartridge can provide spring-mounting or other biasing to the tissue stabilizer to provide a consistent force, thus keeping the tissue taut. Moreover, the cartridge can have one or more force sensors (e.g., four force sensors disposed at the four corners of the cartridge) to measure the force throughout harvesting.

In another example, the cartridge can be a modular component that can be swapped based on characteristics (or parameters) of the harvest site. Thus, a plurality of cartridges can be provided with varying needle and array parameters, based on known differences in harvest site tissue (or recipient site tissue, such as a wound site). For example, the length of the needle (e.g., the one or more extensions at a distal end thereof) or heel-to-tip length can be modified according to patient gender, age, race, skin tightness, and so on. Different cartridges can have larger or fewer numbers of needles in a needle array, while sharing a common cartridge size.

In implementations in which the cartridge is configured to interface with the housing 102, the cartridge and the housing 102 can communicate by any combination of wired and wireless modalities. In one example, the cartridge and the housing 102 can each include an electrode formed thereon, such that when the cartridge is coupled to the housing 102 the electrodes contact one another to exchange information (e.g., or otherwise complete or close a electronic circuit, switch, etc.). In other examples, the cartridge and the housing 102 can communicate wirelessly, for example via a Bluetooth (R) protocol, a near field communication (NFC) protocol, a Wi-Fi® protocol, a proprietary protocol, an optical communication, and the like. The cartridge and the housing 102 can include cooperating ports to provide for physical securement and data transfer. In any of these implementations, the housing 102 can include a reader configured to determine a cartridge type (e.g., by consulting a database or other data structure including a list of known sizes or types of cartridges). In addition to cartridge size, the communicated information can include a determination of whether the cartridge has been used before (and if so, how many times). The uses can be timestamped, and computing device of the system 100 can use one or more thresholds to determine whether or not to “accept” the cartridge. The communicated information can include control signals (e.g., to activate or deactivate a cartridge).

To prevent fluid ingress, in some implementations a flexible membrane can be positioned between the hammer(s) and hollow tubes, such as, as part of the cartridge (e.g., the hollow tubes 104 being part of the cartridge, but the hammer(s) not being part of the cartridge). The flexible membrane can be the same as, or included in addition to, the substrate 2058. The flexible membrane can be formed of any material that is fluid-impermeable, and can be dimensioned such that little or no gap exists between the outer edges of the flexible membrane and the inner edges of the microneedle array 2018, thus preventing the inflow of fluid. The flexible membrane can have a plurality of holes to permit the microneedles 2006 to pass therethrough, again with little or no gap so as to prevent the inflow of fluid.

In some examples, the hollow tube(s) 104 themselves can be modified. For example, hollow tubes 104 (e.g., each being a microneedle) can have a roughened inner diameter to increase friction therein, thus aiding in tissue capture and retention.

In some non-limiting examples, each hollow tube of the hollow tubes 104 can be insertable, such as by the actuation system 110, into tissue (e.g., skin tissue) without rotating each hollow tube (e.g., around a longitudinal axis thereof). Correspondingly, each hollow tube of the hollow tube 104 can be removed from the tissue without rotating the hollow tube. Each hollow tube of the hollow tubes 104 can, then, when removed from the tissue, retain a tissue portion therein (e.g., within a lumen of the hollow tube). This tissue portion is removable from the surrounding tissue by a respective hollow tube without rotating the hollow tube about its longitudinal axis. Thus, in some cases, each hollow tube of the hollow tubes 104 is only translatable (e.g., relative to the housing 102). More specifically, each hollow tube of the hollow tubes 104 is only downwardly translatable and upwardly translatable.

In some non-limiting examples, each hollow tube of the hollow tubes 104 is configured to harvest (e.g., retain) a tissue portion (e.g., a micrograft) when the respective hollow tube 104 is removed from the surrounding tissue. In some cases, at least one tissue portion does not (e.g., all tissue portions do not) include a hair follicle (e.g., any portion of a hair follicle including a hair bulb). Although such a system could be used for harvesting and subsequently implanting hair follicles, the system 100 can be used for procedures where hair follicles are simply not needed to be harvested, or that, in some cases, where the inclusion of hair follicles would be burdensome for the patient or subject at the recipient site. For example, when the recipient site is the face of an individual, tissue portions including hair follicles would be undesirable visually for the patient.

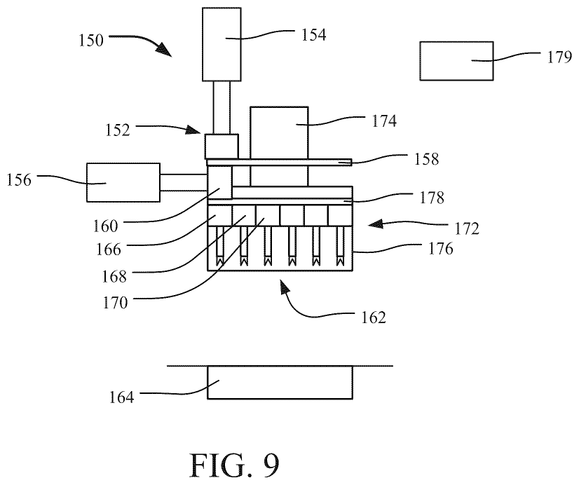

FIG. 9 shows an example of a system 150 for skin grafting and is a specific implementation of the system 100. The system 150, which can be a skin grafting system and can also a simplified configuration of the skin grafting system 3000 to detail an actuation system involved in other portions of the application. Although apparent, the system 150 pertains to the other systems described herein (and vice versa), and as such features, components, etc., of the system 150 can be applied to other systems described herein (and vice versa). The system 150 includes an actuation system 152. The actuation system 152 can include actuators 154, 156, a plunger 158, and a hammer 160, each of which can be similar to the other components of other systems described herein. For example, the actuator 154 can be similar to or implemented as the vertical component assembly 1046, the actuator 156 can be similar to or implemented as the horizontal component assembly 1044, the plunger 158 can be similar to or implemented as the solenoid plunger bars 1106, and the hammer 160 can be similar to or implemented as the horizontal carriage assembly 1082. As detailed below, the actuation system 152 can be configured to translate each hollow tube in the skin tissue site (e.g., a donor skin tissue site). For example, the actuation system 152 can be configured to independently translate specific segments of an array of hollow tubes (e.g., sequentially).

The system 150 can include a plurality of hollow tubes 162, each of which can be implemented in various ways according to this disclosure. For example, each hollow tube can have one or more extensions at a distal end thereof to facilitate insertion of the respective hollow tube into the skin tissue site 164. As a more specific example, each hollow tube can have only two extensions at a distal end thereof, which can be formed by a bevel on opposing longitudinal sides of the hollow tube (e.g., which can also define cutting surfaces and which can be disposed on an exterior surface of the tube). In this way, the bevels can more easily penetrate the skin tissue site 164 when the respective hollow tube is translated into the skin tissue site 164.

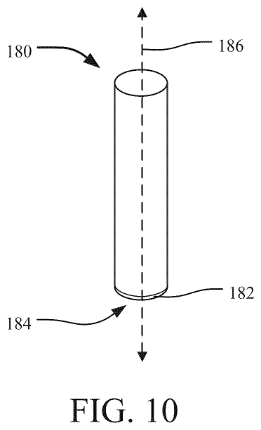

In some non-limiting examples, however, having one or multiple extensions at a distal end thereof, particularly the tubes illustrated with two extensions (each having an exterior bevel) are better suited for solely translation into the tissue. However, in other non-limiting examples where rotating the hollow tubes along its longitudinal axis is desired to cut and sever the portion of skin tissue from the skin tissue site, a hollow tube can be structured differently to align with these aims. To highlight this point, FIG. 10 shows an example of a hollow tube 180. The hollow tube 180 can include a cutting edge 182 positioned at a distal end 184 of the hollow tube 180. The cutting edge 182 can be configured to cut tissue as the hollow tube 180 rotates around the longitudinal axis 186 of the hollow tube 180. The cutting edge 182 can be implemented in different ways. For example, the cutting edge 182 can extend partially or entirely around the longitudinal axis 186 (e.g., the cutting edge 182 can extend an arc length around the longitudinal axis, where the arc length being 360 degrees corresponds to the cutting edge 182 extending entirely around the longitudinal axis 186). As another example, the cutting edge 182 can be formed from an inner bevel (e.g., a bevel directed into the interior surface of the hollow tube 180), and outer bevel (a bevel directed into the exterior surface of the hollow tube 180, as shown in FIG. 10), both an inner and outer bevel, etc. In some cases, the cutting edge 182 being formed from an inner and outer bevel can advantageously allow for a thin, sharper, cutting edge 182. In some cases, the axial cross-section of the hollow tube 180 including the cutting edge 182 at the distal end 184 thereof can be (substantially) circular. In this way, the rotational movement of the hollow tube 180 performs a uniform cut around the tissue, which can avoid deforming, mangling, etc., the tissue (e.g., if the hollow tube 180 were, for example, elliptical, the tissue would be compressed and forced if rotated to cut the tissue). In some cases, the cutting edge 182 can be formed in other ways (e.g., without a bevel) and thus the thickness of the cutting edge 182 decreases as the particular thickness moves away from the proximal end and towards the terminal of the distal end 184 of the hollow tube 180.

As shown in FIG. 10, the hollow tube 180 lacks any extensions at the distal end 184 thereof, which as described above, can be advantageous, particularly for rotational cutting and severing of portions of tissue from the surrounding tissue. Further, in some configurations, the hollow tube 180 can lack a cutting edge 182 (e.g., the hollow tube 180 being a hypotube). In this case, rather, the distal end 184 of the hollow tube 180 can have a uniform thickness. Regardless of the configuration, as the hollow tube 180 rotates into the tissue (e.g., the tissue 164), and in some cases with translation too, the distal end 184 of the hollow tube 180 cuts and severs a portion of the tissue from the surrounding tissue.

Referring back to FIG. 9, each hollow tube of the plurality of hollow tubes 162 can be implemented according to any of the hollow tubes, needles, etc., described herein. For example, each hollow tube can have an inner width, inner diameter (e.g., when the hollow tube has a cylindrical shape), etc., which can receive and retain the portion of skin tissue when the hollow tube is removed from the skin tissue site 164. The inner width, inner diameter, etc., of each hollow tube can be less than or equal to substantially 1 mm, 0.9 mm, 0.8 mm, 0.7 mm, 0.6 mm, 0.5 mm, 0.4 mm, 0.3 mm, 0.2 mm, 0.1 mm etc. In this way, relatively small tissue portions that are harvested are more likely to survive when transplanted to a target site because the relatively small size better facilitates nutrient diffusion (e.g., oxygen). In some specific cases, a dimension of less than 0.6 mm allows for better nutrient diffusion, than say, compared to dimensions of 1 mm or larger. In some cases, a dimension above, for example, 0.1 mm can ensure that the hollow tube maintains its structural integrity when inserted into the skin tissue site 164. In some configurations, an inner surface of the hollow tube (e.g., a wall of the hollow tube, an inner wall of the hollow tube, etc.) can be roughened (e.g., by sandblasting, other abrasion techniques, etc.), which can improve the retention between a captured portion of skin tissue and the hollow tube. In some cases, this roughened portion can extend along a portion or the entire longitudinal length of the hollow tube. Similarly, this roughened portion can extend partially or entirely around the longitudinal axis of the hollow tube. In some cases, the length of the roughened portion can extend partially or entirely along the longitudinal length of the hollow tube. In some cases, the length of the roughened portion corresponds to the length of the portion of skin tissue to be harvested. In some cases, including when the roughened portion does not extend along the entire length of the hollow tube, the remaining portion of the inner surface can be substantially smooth (e.g., not roughened).

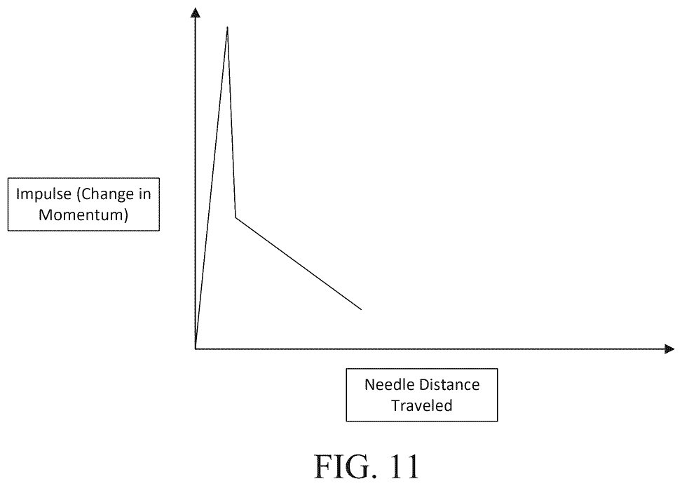

In some configurations, and as shown in FIG. 9, the plurality of hollow tubes 162 can be an array of hollow tubes, such as a 2D array of hollow tubes. In some cases, the array of hollow tubes 162 can include segments 166, 168, 170, with each segment including a plurality of hollow tubes (e.g., of the plurality of hollow tubes 162). Each segment 166, 168, 170 can include a block, substate, etc., that is coupled to the plurality of hollow tubes. In this way, as the block moves, so does the plurality of hollow tubes coupled thereto. As shown in FIG. 9A, each segment 166, 168, 170 includes a single linear row of hollow tubes (e.g., two, three, four, five, etc.). However, in other configurations, each segment 166, 168, 170 can include other numbers and configuration of hollow tubes. For example, each segment 166, 168, 170 can include multiple rows of hollow tubes (e.g., a block of hollow tubes), a single or multiple rows of columns of hollow tubes (e.g., a block of hollow tubes), multiple hollow tubes in the form of a segment of a circle (e.g., a pie segment, such as, when the 2D array of hollow tubes is implemented as having a circular peripheral shape), etc. Regardless of the configuration, each segment 166, 168, 170 can be translated independently (e.g., by the actuation system 152), such that each segment 166, 168, 170 is only translated at one time along with its corresponding plurality of hollow tubes coupled therewith into the skin tissue site 164. In some cases, this independent translation can occur sequentially (e.g., the segment 166, followed by the segment 168, followed by the segment 170, and so on) or can occur in a different order (e.g., the segment 166, followed by the segment 170, followed by the segment 168). In some configurations, although three segments have been denoted in FIG. 9A, it is appreciated that the plurality of hollow tubes 162 can have various numbers of segments (e.g., two, three, four, five, six, seven, eight, nine, ten, etc.) with six segments being illustrated in FIG. 9A. In some cases, each segment 166, 168, 170 can have different numbers of hollow tubes or the same number of hollow tubes. In some configurations, independent translation of each segment (as opposed to multiple segments together or the entire array) can advantageously mitigate insertion forces while keeping a relatively large sized array of hollow tubes (e.g., by inserting a manageable number of hollow tubes at one time, the size of the array becomes largely irrelevant with respect to insertion forces). Further, not only is the amount of force important for tube insertion, but also the force over a given amount of time (i.e., the impulse). FIG. 11 shows a graph illustration the relationship between the impulse and the distance the needle travels into the tissue. The peak impulse represents the impulse required to puncture the epidermis, which is the part of skin tissue that is most resistant to puncturing or translation. After the epidermis has been punctured, the amount of force required to move the needle a further distance into the tissue is significantly decreased. Accordingly, and referring back to FIG. 9A, segments are far easier to generate the requisite impulse needed to insert the tubes into the skin tissue site 164 as compared to larger numbers of tubes (e.g., the entire array). In this case, as described below, the actuator 154 can be configured to quickly generate the force needed to provide the relatively large impulse required to insert each segment 166, 168, 170 and its respective hollow tubes into the skin tissue site 164. Accordingly, as described below, the actuator 154 can be electrical actuator (e.g., a solenoid), a pneumatic actuator, etc. In some cases, then, the system 150 (or the system 100 and in particular the actuation system 110) can be configured to provide a force of at least substantially 1.5 lbs., 2 lbs., 2.5 lbs., etc., per needle (e.g., force per needle in a segment) to penetrate the epidermis. Correspondingly, the system 150 (and the system 100) can be configured to translate the one or more hollow tubes of a segment (e.g., each segment) at a speed of at least substantially 0.4, 0.5, 0.6, 0.7, 0.8 meters per second. The speed and the force provide the impulse for penetrating the epidermis and other portions of the tissue.

In some configurations, and as illustrated in FIG. 9, the plurality of hollow tubes 162 can be implemented as a cartridge 172 that is removably coupled to a housing 174 of the system 150, which can be in a similar way as the other cartridges and housings described herein. For example, the cartridge 172 can include a peripheral housing 176 that can surround the plurality of hollow tubes 162. Further, as described in other configurations, each segment 166, 168, 170 and corresponding plurality of hollow tubes can be slideably engaged to the cartridge 172, and more specifically, to the peripheral housing 176 of the cartridge 172. In some non-limiting examples, in a similar way to the other configurations, each hollow tube of the plurality of hollow tubes 162 can include a corresponding pin that can facilitate harvesting of a portion of skin tissue and subsequent depositing of the portion of skin tissue from the hollow tube.

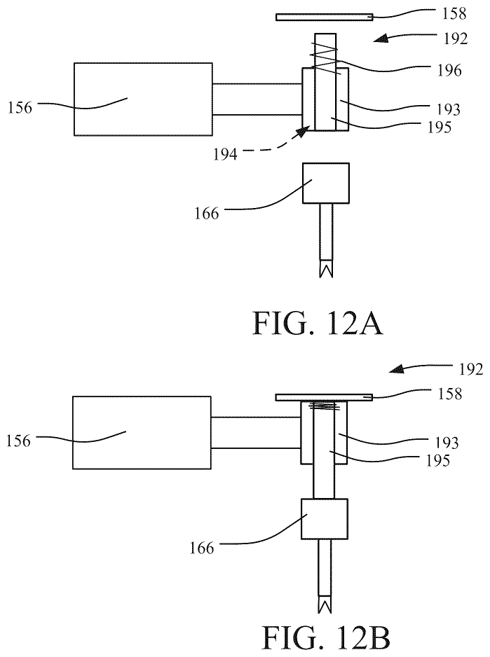

As shown in FIG. 9, the actuation system 152 can include the hammer 160 that is configured to drive each segment of the plurality of hollow tubes 162 into the skin tissue site 164, which can be implemented in a similar way to the horizontal carriage assembly 1082 and corresponding hammers 1098a, 1098b. For example, as shown in FIG. 9, the hammer 160 can be coupled to the actuator 156, such that movement of the actuator 156 moves the hammer 160. In some cases, the hammer 160 can include a spring (or multiple springs, such as the springs 1092a, 1092b) that can force the hammer 160 back into position after being driven. For example, the actuator 156 can move the hammer 160 into a position in which the hammer 160 is aligned with the segment 166 (e.g., and only the segment 166). Then, the actuator 154 can “fire” or otherwise drive the plunger 158 (e.g., which can be coupled to an end of the actuator 154) into the hammer 160 thereby driving the segment 166 and its plurality of hollow tubes into the skin tissue site 164. As the plunger 158 drives the hammer 160, the spring of the hammer 160 loads. At this point, the actuator 154 retreats (e.g., upwardly) thereby retreating the plunger 158. As the actuator 154 retreats, the spring of the hammer 160 unloads and brings the hammer 160 back above the other segments of the array. This process can define one cycle, and this same cycle can be completed for as many cycles as desired, such as, until all the segments have been inserted into the skin tissue site 164. For example, the second cycle can begin with the actuator 156 moving the hammer 160 to a second position above the segment 168. To illustrate this point, FIGS. 12A and 12B show an example of a hammer 192, which can be implemented as the hammer 160.

The hammer 192 can include a support 193 having a hole 194 directed therethrough, a driver 195, and a spring 196. The support 193 can be implemented in different ways to provide rigidity to the hammer 192. For example, the support 193 can be implemented as a block of material (e.g., a polymer, such as a plastic), a cylinder of material, etc. Further, as described above, the support 193 can have the hole 194 directed therethrough, which can provide a location for the support 193 to receive the driver 195. As detailed below, the driver 195 can be positioned within the hole 194 and can be coupled to the support 193. For example, as shown in FIG. 12A, the spring 196 is coupled to the support 193 and the driver 195 (e.g., coupled between the support 193 and the driver 195). In some configurations, and as illustrated, the spring 196 surrounds the driver 195 (e.g., an end of the driver 195) and can be concentrically arranged relative to the driver 195. The driver 195 can be shaped in different ways. For example, the driver 195 can have the same or similar shape (e.g., a cylinder) as the shape of the substrate of the segment 166 (e.g., which can be the same shape as the other remaining substrates of the array). As another example, although not illustrated in FIG. 12A or 12B, the area of the driver 195 that contacts the substate of the segment 166 can be substantially the same, so as to better transfer the force provided to the driver 195. In this regard, the hole 194 of the support 193 can have an area (e.g., a cross-sectional area) that is substantially the same as the area of the substrate of the segment 166 (e.g., these areas being in alignment with each other).

In some non-limiting examples, and as illustrated in FIG. 12A, a portion of the driver 195 (e.g., a top of the driver 195) can extend above the support 193 a distance. This distance can correspond to the maximum penetration distance of the plurality of hollow tubes of the segment 166 (and others within the array), or stated differently, the maximum translation distance of the plurality of hollow tubes (e.g., which can be substantially less than 10 mm, 9 mm, 8 mm, 7 mm, 6 mm, 5 mm, 4 mm, 3 mm, 2 mm, etc. For example, FIG. 12B shows the driver 195 in a second position (e.g., an actuated position) opposite the first position (e.g., a resting potion) as illustrated in FIG. 12A. In particular, as the plunger 158 advances (e.g., from an actuator), the plunger 158 contacts a top end of the driver 195 to translate the opposing bottom end of the driver 195 (e.g., downwardly). Correspondingly, the bottom end of the driver 195 contacts the substate of the segment 166 and drives the segment 166 into the skin tissue site. Once the plunger 158 contacts the support 193 (e.g., a top of the support 193), the plunger 158 stops advancing. During movement of the driver 195, the spring 196 is biased or otherwise loaded (e.g., the spring 196 is illustrated as a compression spring, but other spring configurations are possible including using extension springs, tortional springs, etc.). Therefore, when the plunger 158 is retreated (e.g., upwardly), the driver 195 also translates back towards the first position (e.g., the resting position). In some configurations, this hammer 192 configuration can provide for insertion of different segments of the array, irrespective of a predefined order. For example, in some cases, each segment of the array can include mechanical stops that block further advancement of the hammer 192. While this can be helpful in some configurations, such as to provide assurance that the particular segment has been inserted, without these mechanical stops, the hammer 192 can be advanced to any position. In this way, insertion order does not have to be sequential (e.g., first segment followed by the second segment and so on), but rather can be any desired order. Further, as described below, other sensing configurations can indicate when a particular segment of the array has been inserted into the skin tissue site so as to mitigate any undesirably associated with removal of the mechanical stops.

Referring back to FIG. 9, as shown in this figure the system 150 can include a membrane 178 that can extend above all the plurality of hollow tubes 162. Similarly, the membrane 178 can extend below the hammer 160. In some cases, the membrane 178 can provide a seal so as to mitigate fluid ingress into the actuation system 152, the housing 174, and other portions of the system 150. For example, during translation of hollow tubes into skin tissue can release blood, bodily fluids, and other contaminants. These possible contaminants in some cases, such as without the membrane 178, can disperse into those components thereby contaminating them. This makes it difficult for the actuation system 152, housing 174, and other portions of the broader system 150 to be used with other plurality of hollow tubes (e.g., other cartridges). The membrane 178 positioned between the plurality of hollow tubes 162 and the hammer 160 can prevent these possible contaminants from being forced upwardly, while at the same time the flexibility of the membrane 178 can ensure that the hammer 160 does not puncture the membrane 178 during driving of the hammer 160 into a segment. In some cases, therefore, the cartridge 172, which can include the plurality of hollow tubes 162, can also include the membrane 178. In this case, the membrane 178 can be positioned within the peripheral housing 176 of the cartridge. Further, the membrane 178 can be coupled to opposing ends of the peripheral housing 176 and can extend past all the plurality of hollow tubes 162. Although the membrane 178 has been described as being flexible and providing a seal, in other configurations, the membrane 178 can be a diaphragm, a sheet, etc., which can have similar properties as the membrane 178. In some cases, the membrane 178 can be formed of different materials including polymers (e.g., a plastic, a rubber, etc.).

As shown in FIG. 9, the system 150 can include a computing device 179 and can be implemented as the other controllers, computing devices, processors, etc., described herein. The computing device 179 can be separate from the other components of the system 150 (e.g., a standalone device, such as, for example, a smartphone, computer, etc.) or can be coupled to one or more components of the system 150. For example, the computing device 179 can be coupled to the housing 174 of the system 150. The computing device 179 can be in communication with various components of the system 150 and can control, move, etc., each component that the computing device 179 is in communication with. For example, the computing device 179 can be in communication with the actuation system 152 (including its subcomponents) and can cause components of the actuation system 152 to move (e.g., the actuators 154, 156). In some configurations, the computing device 179 can be a computing device or can include one or more computing devices (e.g., multiple computing devices).

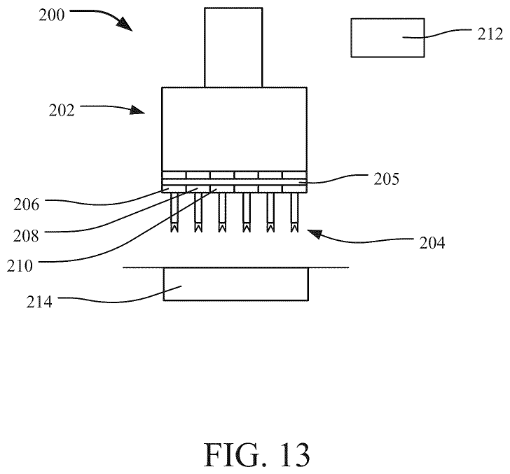

FIG. 13 shows an example of a system 200, which can be a specific implementation of the system 100. The system 200, which can be a skin grafting system, can also a simplified configuration of the skin grafting system 3000 to detail an actuation system involved in other portions of the application. Although apparent, the system 200 pertains to the other systems described herein (and vice versa), and as such features, components, etc., of the system 200 can be applied to other systems described herein (and vice versa), e.g., the system 150. The system 200 can include an actuation system 202 (e.g., which can be the same or similar as the actuation system 152), a plurality of hollow tubes 204 (e.g., which can be the same or similar as the plurality of hollow tubes 162 including the cartridge 172), and a rotator 205. Similarly to the system 150, the plurality of hollow tubes 162 can have multiple segments, each having a substrate coupled to a plurality of tubes. Further, the plurality of hollow tubes 162 can be formed as an array (e.g., an array of hollow tubes). As shown in FIG. 13, the system 200 can include segments 206, 208, 210, each of which can include a substrate with a plurality of tubes coupled thereto. Further, similarly to the system 150, the system 200 can include a computing device 212.

The rotator 205 can be configured to rotate one or more of the plurality of tubes 204, where rotation can be around a longitudinal axis of the respective hollow tube (e.g., in a clockwise or counterclockwise direction). In some configurations, as described in more detail below, the actuation system 202 can translate a segment 206 into the tissue site 214, referred to later as a skin tissue site, as the rotator 205 rotates one or more hollow tubes within the segment 206. This can be completed for each segment as the actuation system 202 translates the respective segment. In this way, the hollow tube can sever the surrounding tissue as it rotates and translates, in a corkscrew-like manner, which can make insertion of the hollow tube into the skin tissue site 214 easier (e.g., requiring less overall force, less impulse, etc.). In other words, rotation of the hollow tube cuts the surrounding tissue as the hollow tube rotates and translation of the hollow tube into the surrounding tissue also cuts the surrounding tissue. To that end, and as descried in more detail below, using a rotator, like the rotator 205, can decrease the required speed of the actuator that drives a plunger (e.g., the actuator 154), due to the decrease in force requirements. In this regard, the actuator is allowed to translate or be driven more slowly, which can advantageously decrease the power requirements (e.g., from the decrease in current draw) of the actuator and broader system 200. In some cases, the rotator 205 can rotate one or more of the hollow tubes of the segment 206 until the specific hollow tube penetrates the epidermis of the skin tissue site 214. In this way, the rotation of a hollow tube can help overcome the relatively high penetration impulse required when the hollow tube is only translated into the skin tissue site (e.g., rotation helps sever the tissue where it is needed most). In some cases, the computing device 212 can cause the rotator 205 to rotate the one or more hollow tubes of the segment 206 during a period of time which includes penetration of the skin tissue 214.

In other configurations, the rotator 205 can rotate one or more (e.g., all) hollow tubes of the segment 206, after the segment 206 has been translated completely into the skin tissue site 214 (e.g., the segment being translated the maximum distance). In this way, rotation of the hollow tubes can ensure that a given hollow tube has adequately severed the portion of the skin tissue from the skin tissue site 214 before the hollow tube is retracted. In some configurations, each hollow tube of the plurality of hollow tubes 204 of the system 200 can be configured in various ways, such as the configuration of the hollow tube 180 of FIG. 10, the configuration of the hollow tube of 2054 of FIGS. 6A and 6B, etc. In some cases, using a hollow tube configured like the hollow tube 180 can be advantageous because the cutting edge 182 being curved can more easily cut or sever the surrounding tissue when the tube rotates.

In some configurations, the rotator 205 can rotate each hollow tube of the plurality of hollow tubes 162 in a first direction (e.g., counterclockwise) at a first speed (e.g., a constant speed, a variable speed, etc.). However, in other configurations, the rotator 205 can rotate each hollow tube of the plurality of hollow tubes 162 in a first direction (e.g., clockwise), can reverse the rotational direction to a second direction (e.g., counterclockwise), and can reverse the rotational direction back to the first direction, and so on. In some cases, this can provide oscillatory (or vibratory) movement, which can allow for better penetrating of the skin tissue.

In some configurations, a rotational mechanism, such as the rotator 205 that is configured to rotate one or more of the plurality of hollow tubes, each about its central axis, can provide sufficient force for penetrating and cutting the biological tissue (e.g., skin tissue), and thus each hollow tube can be non-beveled (e.g., may not include the plurality of extensions at the distal end thereof). In other configurations, each hollow tube 104 can have one or more cutting edges, such as a back bevel, a bias bevel, a hypodermic configuration, a franseen (e.g., including a plurality of external cutting edges), etc., to facilitate rotational severing of the portion of skin tissue from the skin tissue site. In some cases, in addition to the one or more external bevels (e.g., as illustrated in FIG. 6A), each hollow tube can include one or more additional cutting surfaces (e.g., a rotational cutting surface, a cutting edge, surface, etc.) that extends along a longitudinal axis of the respective hollow tube). In this way, as a hollow tube is rotated (e.g., alone its longitudinal axis), the hollow tube can better penetrate the tissue. In some configurations, each hollow tube can include a flat tip, such as a substantially blunt tip (i.e., generally cylindrical at the end). In some cases, each hollow tube can be formed of a metal material, such as stainless steel, and can be treated with a finishing process such as electropolishing or passivation to improve corrosion resistance or to remove surface defects or impurities. Therefore, each hollow tube can be electro-polished, can be a hypotube, and can lack any exterior or interior bevels.

In some configurations, a portion of the rotator 205 (e.g., a belt, band, chain, etc.) can surround one or more of the hollow tubes and can be positioned within a peripheral housing of a cartridge. For example, a portion of the rotator 205 can surround a substrate of one or more of the segments (e.g., the segment 206). In some cases, the portion of the rotator 205 can be positioned within a channel of each substrate of each segment.

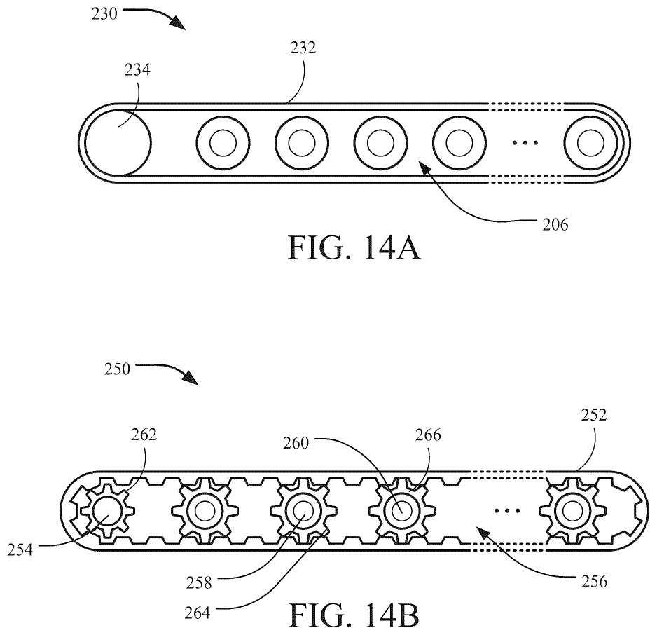

FIG. 14A shows an example of a rotator 230, while FIG. 14B shows an example of a rotator 250, each of which can be specific implementations of the rotator 205. The rotator 230 can include a belt 232 (e.g., a rubber band, rubber belt, elastic belt, etc.) and a drive 234. As shown in FIG. 14A, the segment 206 of hollow tubes (e.g., that includes a plurality of hollow tubes) is shown as a single linear row of hollow tubes, with the belt 232 surrounding the hollow tubes and with the drive 234 in alignment with the hollow tubes (e.g., however, the drive 234 can be positioned out of alignment with the hollow tubes). The drive 234, which can be a driveshaft, a motor, etc., can cause or otherwise drive movement of the belt 232 thereby rotating all the hollow tubes in the segment 206. For example, the drive 234 and each hollow tube of the segment 206 can be forced into contact (e.g., frictionally) with a portion of the belt 232, such that, as the drive 234 rotates and moves the belt 232 (e.g., translates the belt), the belt 232 rotates the hollow tubes. In some cases, the drive 234 can rotate continuously in a first direction (e.g., counterclockwise) at a constant speed (e.g., or variable speeds). However, in other configurations, the drive 234 can rotate in a first direction (e.g., clockwise), can reverse the rotational direction to a second direction, and back, and so on. In some cases, this can provide oscillatory movement, which can allow for better penetrating of the skin tissue.

The rotator 250 can include a chain 252 and drive 254. As shown in FIG. 14B, a segment 256 that includes a plurality of tubes including hollow tubes 258, 260. Similarly to FIG. 14A, the segment 256 can be a specific implementation of the segment 206. In some cases, and as illustrated, the chain 252 surrounds the segment 256 and the plurality of tubes. The drive 254 can include a gear 262, while the hollow tubes 258, 260 can include respective gears 264, 266. Each gear 262, 264, 266 can include one or more teeth or protrusions and can be coupled to or integrally formed with the underlying component. The drive 254, which can be a driveshaft, motor, etc., can cause or otherwise drive movement of the chain 252 by the teeth of the gear 262 engaging with and driving the chain 252 (e.g., when the teeth are positioned within holes in the chain 252). Likewise, as the chain 252 moves (e.g., translates), the chain 252 causes each tube of the plurality of tubes of the segment 256 to rotate (e.g., by the teeth of the gear 264, 266 of the hollow tubes 258, 260) around its longitudinal or central axis. Similarly to the rotator 230, the rotator 250, including the drive 254, can rotate in a first rotational direction at a constant (or variable speed), or can provide oscillatory movement in a similar way as the rotator 230 (e.g., by the drive 254 switching rotational directions). Although gears have been described with respect to the rotator 250, in other configurations, one or more protrusions or teeth can replace the corresponding gear to be inserted into and engage with the chain 252.

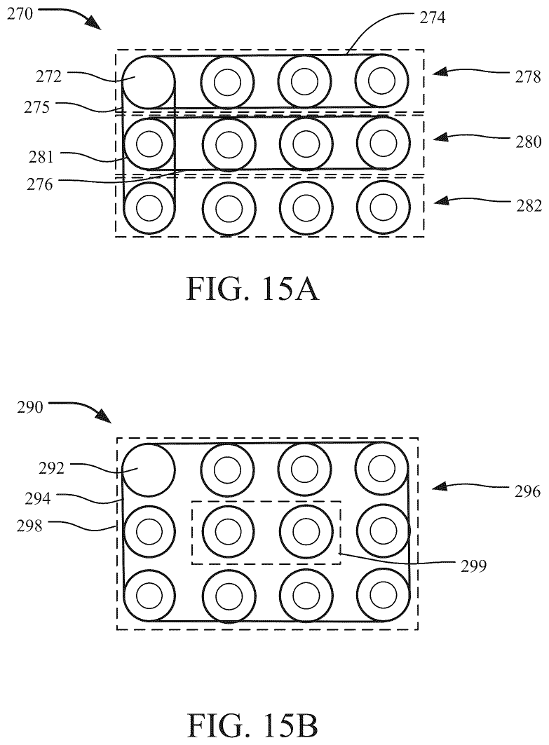

In some cases, in either of FIG. 14A or 14B, a plurality of segments (e.g., rows) of hollow tubes (e.g., the entire array 2006) can be linked, coupled, etc., to one another. For example, one or more linkages can be provided between multiple drives, corresponding to each segment (e.g., each row that can be a single linear row), or a single drive can be connected by one or more linkages to each segment (e.g., by multiple belts, chains, etc.). For example, FIG. 15A shows a rotator 270 with a drive 272, and rotational couplers 274, 275, 276 (e.g., a chain, a band, a belt, etc.) relative to a plurality of hollow tubes in segments 278, 280, 282 (e.g., each shown as a single row of hollow tubes). The drive 272, which can be the single drive in the respective system to drive rotation of the tubes, rotates in a first rotational direction, thereby causing movement of the rotational couplers 274, 275, 276, thereby causing the hollow tubes of the respective segment 278, 280, 282 to rotate in the same (or opposite direction). For example, rotation of the drive 272 can cause the rotational coupler 275 to move, which causes the hollow tube 281 of the segment 280 to rotate. This rotation of the hollow tube 281 then drives movement of the rotational coupler 276, which then drives rotation of the other tubes in the segment 280. Although a single drive 272 can drive rotation of all of the hollow tubes (e.g., different segments of hollow tubes, including the segments 278, 280, 282), in other configurations, each segment of the plurality of hollow tubes (e.g., an array of hollow tubes) can include a respective drive (e.g., implemented in a similar way as the drive 272). In this way, rotation of the drive of a segment can drive rotation of only the hollow tubes in that segment, which can allow for independent rotation of segments. For example, during translation or actuation of a given segment of hollow tubes, rotation of only the given segment can be desirable (e.g., rather than the other hollow tubes together) because rotation can aid in insertion of those tubes. In other words, rotating tubes that are not currently being advanced, translated, actuated, etc., can be a waste of power, and could be undesirable due to inadvertent contact with other portions of the body (e.g., not during harvesting). Although the segments 278, 280, 282 have been shown as being rows of hollow tubes, in other configurations, each segments 278, 280, 282 can be a column of hollow tubes (e.g., a single linear column of hollow tubes). Further, in some configurations, hollow tubes can drive rotation of adjacent hollow tubes, such as those in the same segment, by for example, engagement between respective gears. More specifically, with reference to segment 278, the drive 272 can drive rotation of the adjacent hollow tube in the segment 278 (e.g., by engagement between them). This adjacent hollow tube rotates and correspondingly drives rotation of an adjacent hollow tube of the segment 278, which can occur until each hollow tube in the segment 278 rotates. Thus, the drive 272 can drive rotation of hollow tubes without a rotational coupler.

FIG. 15B shows an example of a rotator 290 with a drive 292, and a rotational coupler 294 (e.g., a chain, a band, a belt, etc.), relative to a plurality of hollow tubes 296. The plurality of hollow tubes 296 are separated into peripheral tubes 298 (e.g., or in other words outer tubes) and inner tubes 299. The peripheral tubes 298 are hollow tubes that are positioned on the outside perimeter of the array, whereas the inner tubes 299 are positioned internally in the array, internal to the peripheral tubes 298. As shown in FIG. 15B, each of the peripheral tubes 298 can be in contact with a rotational coupler 294, with the rotational coupler 294 also being in contact with the drive 292. Although not shown in FIG. 15B, each inner tube 299 can be in contact with either a corresponding inner tube 299 or a peripheral tube 298. In some cases, at least one inner tube 299 is in contact or engagement with a peripheral tube 298. In this way, rotation of the drive 292 can drive movement of the rotational coupler 294, which rotates the peripheral tubes 298. Rotation of peripheral tubes 298 is then transmitted to the inner tubes 299 to drive rotation of those tubes. In some configurations, as described above, rather than having a rotational coupler 294 (or to provide redundancy, including providing consistent rotational speed, ensuring that all the tubes actually rotate, etc.), the peripheral tubes 299 can drive rotation of adjacent peripheral tubes 299 (e.g., by engagement between respective gears).

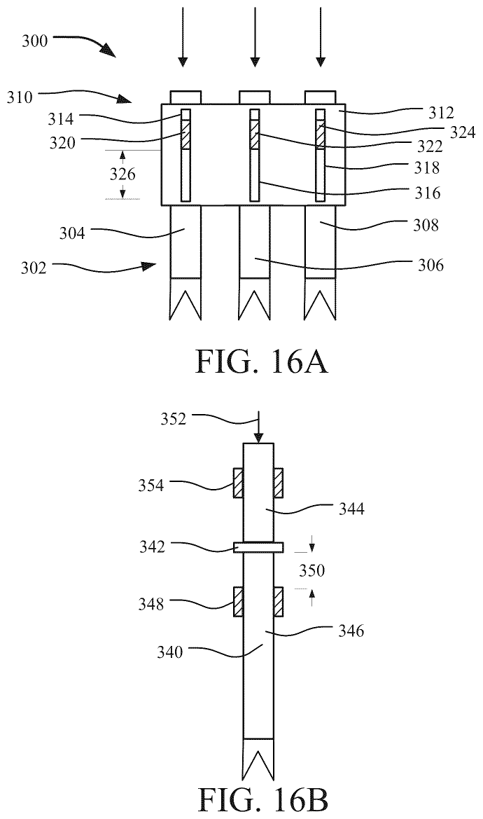

FIG. 16A shows an example of a simplified illustration of a system 300, which can be a skin grafting system, showing a specific implementation of a rotator. The system 300 can include a segment of hollow tubes 302 (e.g., shown in FIG. 16A as being single row of three hollow tubes) including individual hollow tubes 304, 306, 308, and a rotator 310 including a rotational coupler 312. The rotational coupler 312 can be implemented in various ways including being a chain, a belt, a band, etc. The rotational coupler 312 can include slots 314, 316, 318 each of which are configured to receive a corresponding protrusion 320, 322, 324. Although these slots 314, 316, 318 are shown as being rectangular other shapes are possible. For example, the slots can be channels, recesses, etc. Similarly, although the protrusions are 320, 322, 324 are shown as having a similar (or the same) shape as the respective slots 314, 316, 318, such as being rectangular, the protrusions 320, 322, 324 can have other shapes. For example, each protrusion can be a tooth (e.g., a tooth of a gear). As shown in FIG. 16A, each slot 314, 316, 318 can permit translation of the respective hollow tube, while ensuring that the respective tube can be rotated. And in some cases the slot can limit the translation distance of the tube. For example, a travel distance 326 (e.g., an axial travel distance) can be defined between an end of the slot 314 and a corresponding end of the protrusion 320. The hollow tube 304 can be translated (e.g., downward towards tissue) until the protrusion 320 contacts an end of the slot 314 at which point further translation of the hollow tube 304 is prevented or otherwise blocked. In this way, the rotational coupler 312 can permit translation while allowing rotation of the hollow tube (e.g., after the hollow tube has been translated, so as to ensure that the micrograft has been adequately severed from the surrounding tissue).

FIG. 16B shows an example of a hollow tube 340 without a rotator shown, but demonstrating another configuration of translation and rotation of a hollow tube 340. The hollow tube 340 can include a flange 342 that can separate the hollow tube 340 into a top section 344 and a bottom section 346, where the bottom section 346 can include a distal end that is configured to penetrate tissue. In some cases, a system (e.g., a skin grafting system) including a component thereof, such as a portion of the cartridge can include a mechanical stop 348 that can at least partially (or entirely) surround the hollow tube 340 (e.g., at the bottom section 346). The mechanical stop 348 can be fixed in place (relative to the hollow tube 340) so as to provide a travel distance 350 (e.g., an axial travel distance). This travel distance 350 can be the maximum travel distance (e.g., translation distance) that the hollow tube 340 is permitted or otherwise is allowed to travel. For example, as the hollow tube 340 is translated in a direction 352 towards the tissue, the flange 342 contacts or otherwise abuts against the mechanical stop 348 to prevent further downward translation of the hollow tube 340 past the mechanical stop 348. In some cases, this flange 342 can advantageously allow a translation distance limit, but also can provide a separation for engagement with a rotational coupler that can engage the top section 344 of the hollow tube 340 (e.g., rotational components are more desired to be on the top section 344 as opposed to the bottom section 346 because the rotational components would be closer to power source, other mechanical engagements such as a drive shaft, etc.). For example, as shown in FIG. 16B, a rotational coupler 354 can engage and surround the top section 344 of the hollow tube 340. Further, the hollow tube 340 can be slidably engaged with the rotational coupler 354, such that the hollow tube 340 can be rotated during translation of the hollow tube 340 (and vice versa). In this case, as the tube 340 translates downward along the direction 352, the top section 344 of the tube 340 advances through the rotational coupler 354 while the rotational coupler 354 rotates the hollow tube 340 (about the longitudinal axis), until, for example, the flange 342 contacts the mechanical stop 348. Although FIG. 16B has been described with respect to a flange 342, in other cases, the flange 342 can be a protrusion, or other shaped component, that engages with the mechanical stop 348 to prevent further downward translation of the hollow tube 340. In addition, this configuration can be implemented for each hollow tube of a plurality of hollow tubes of a system.

In some non-limiting examples, the rotational couplers include a belt and a chain, each of which can extend around a given set of hollow tubes, in some implementations, the segment of hollow tubes can be coupled via a longitudinal member, such as a frictional rack or a toothed rack, as in a rack-and-pinion mechanism to otherwise drive rotation of one or more of the hollow tubes (e.g., when the hollow tubes include gears, teeth, protrusions, etc.). In still other implementations, columns of hollow tubes (or lines in an oblique direction) can be actuated together instead of rows.

Although single linear rows of hollow tubes have been described, in other configurations, segments of hollow tubes can be rotated within the microneedle array. In this case, a plurality of hollow tubes can be rotated together, which can define the segment of hollow tubes. This segment can have various peripheral shapes, including, for example, a circle, a square, a rectangle, a row of hollow tubes (e.g., a single row of tubes), a column of hollow tubes (e.g., a single column of hollow tubes), etc. In this case, the belt, chain, etc., can surround the segment and rotation of the belt, chain, etc., can rotate all or some of the hollow tubes within the segment. In some configurations, each hollow tube that is interior to the peripheral shape of the segment (e.g., a hollow tube internally positioned within the segment, such that the hollow tube does not directly contact the belt, chain, etc.), can include one or more gears, threads, teeth, gears, etc., on an exterior surface. Further, each hollow tube that defines the periphery of the segment can include corresponding (or in other words complementary) gears, threads, etc. In this way, as the belt, chain, etc., directly rotates the hollow tubes on the periphery of the segment, rotation of the peripheral hollow tubes directly rotates the interior tubes as well. Therefore, in some configurations, each hollow tube within the array (e.g., microneedle array) can include one or more threads, gears, etc., to rotate adjacent hollow tubes. In this way, a single belt rotating a group of hollow tubes can cause rotation of all the hollow tubes within the array. In some cases, a segment of hollow tubes can be a row of hollow tubes, a column of hollow tubes, a block of hollow tubes (e.g., a rectangular or square array of hollow tubes), etc. In some configurations, two given segments of the plurality of hollow tubes can have their hollow tubes rotate in the same direction (e.g., hollow tubes of one segment rotating clockwise and hollow tubes of another segment also rotating clockwise), while two given segments of the plurality of hollow tubes can have their hollow tubes rotate in the opposite direction (e.g., hollow tubes of one segment rotating counterclockwise and hollow tubes of another segment rotating clockwise).

In some configurations, a portion of each hollow tube that engages with the rotational drive (e.g., a belt, a chain, etc.) which can be a gear, a thread, etc., can extend an axial distance along the respective tube greater than or equal to 25%, 50%, 75%, or 100% (e.g., the entire) distance that the hollow tube axially translates. In this way, as the hollow tube axially translates, the portion of the hollow tube can still be in contact with the rotational drive to ensure that the hollow tube can be rotated while the hollow tube translates (or after translation has finished). Similarly, each hollow tube can be slideably engaged with a respective gear, with the gear including a mechanical stop. In this way, each hollow tube can be translated downwardly until the hollow tube contacts the mechanical stop. At this point, the rotational drive can rotate the applicable hollow tubes after the hollow tubes have been translated.

In some configurations, the rotational member can be implemented as a belt, a chain, etc. In some cases, the belt can include one or more holes, each of which can receive one or more protrusions of a gear of hollow tube. In this way, the engagement between a protrusion and a hole of the belt can rotate the respective hollow tube. In some cases, the drive can be implemented in different ways. For example, the drive can be a drive shaft that engages with a motor physically (e.g., the drive shaft being engaged with the motor), or magnetically (e.g., as in a magnetic coupling to transfer torque from the motor to the drive shaft magnetically). In some cases, the motor can be an electric motor, a rotor having a permanent magnet, etc. In some configurations, the drive be complementary with a drive of the broader system. For example, a first drive can be positioned in a cartridge (that includes a plurality of hollow tubes) and a second drive can be positioned in the housing (e.g., of a handheld device). In this way, torque provided to the first drive can be transmitted to the second drive, where the first drive can be reused and the second drive can be disposed of along with the cartridge. In some cases, this is particularly desirable for non-physical torque transmission (e.g., where the drives are magnetic couplers) since this further removes the possibility of contamination of the first drive by a physical connection to the second drive (e.g., with bodily fluids including blood).

In certain implementations, the plurality of hollow tubes (e.g., microneedle array 2006) can be actuated (e.g., caused to move in a longitudinal direction thereof, including translation, translation without rotation, downward translation, etc.) by an actuation mechanism other than the solenoid 1052 (e.g., the actuation mechanism being used in place of the solenoid). For example, in implementations which require less force on the donor site (e.g., where segments of an array of hollow tubes, such as a microneedle array 2006 are sequentially actuated, or where the hollow tubes of FIG. 9A or 9B are used), the force (or impulse) can be reduced to a point where a solenoid is not necessary and thus other types of actuation systems can be implemented. These can correspond to the actuation system 110 of FIG. 8.

In some non-limiting examples, the plurality of hollow tubes (e.g., the microneedle array 2006) can be actuated by one or more reloadable springs (e.g., a single relatively large reloadable spring, or multiple reloadable springs, each corresponding to a particular segment of hollow tubes, etc.). In such implementations, each reloadable spring can be configured to operate a segment or group of hollow tubes (e.g., microneedles 2050, a section of the microneedle array 2006, a row or column of microneedles 2050, a row or column of hollow tubes, a segment of hollow tubes, and so on). The reloadable springs can be configured for manual or automatic reloading. In some cases, a motor that requires less energy, power, etc. (e.g., less current draw) than a solenoid, such as DC motor (e.g., a brushless DC motor) that can be implemented as a linear actuator, can be advantageously used along with a reloadable spring. In some configurations, such as the one described above, because of the material properties of skin and particularly the epidermis, needles need to be inserted quickly, which typically requires large forces/impulses. In this case, rather than using a solenoid to deliver the large force, a motor can more slowly load the spring, which can then be released to quickly free the stored spring energy thus quickly insert a segment of hollow tubes. Therefore, the reloadable spring can be biased by the motor by extending a nut that loads the reloadable spring as the nut extends along the screw (e.g., of a linear actuator). Once the reloadable spring is biased to the desired position, the system can prevent premature releasing (e.g., unloading) of the reloadable spring, by for example, activating a stop, lock, brake, etc., which can prevent the reloadable spring from driving a group of needles down prematurely (or otherwise at an undesired time). This can be desirable, for example, just prior to the harvesting procedure (e.g., when the housing, such as the peripheral housing of the cartridge is not in engagement with the donor site). Then, once the system is in the desired place (e.g., being pressed against a donor site with the requisite force), and a component that aligns or causes a particular segment of hollow tubes to be the only ones within the array to be able to translate downwardly (e.g., the horizontal cartridge, as described above, aligning the hammers to the desired row of needles), the stop, lock, brake, etc., can be released to cause the spring to transmit the released spring energy thereby driving and translating the segment of hollow tubes (e.g., a row of hollow tubes) into the donor site. After the first segment of hollow tubes have been inserted, the process can proceed again, with loading of the reloadable spring, preventing of the unloading of the reloadable spring, alignment of the segment selector to only allow that segment to translate (e.g., downwardly), and unloading of the reloadable spring to drive the selected segment of hollow tubes. Obviously, this configuration takes additional time because of the time needed to reload the spring, which can increase the time needed to complete the procedure. However, this configuration can advantageously decrease the footprint or size requirements of the system because the relatively large solenoid can be removed. Further, this configuration, as opposed to the solenoid configuration that requires large continuous current draws, can be better suited for more mobile configurations, such as cordless battery pack configuration (e.g., the power requirements are more minimal as compared to the solenoid configuration).

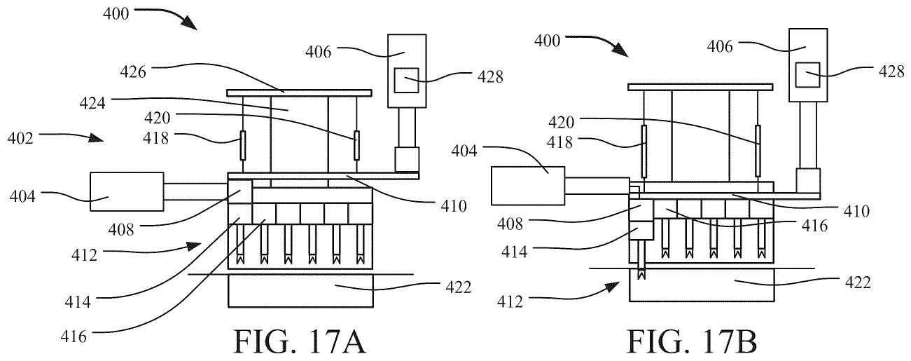

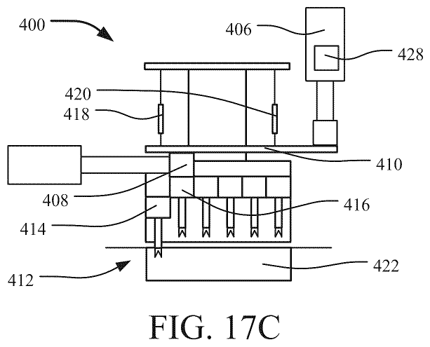

FIGS. 17A-C show an example of a system 400 (e.g., a skin grafting system), which can be a specific implementation of other systems described herein. For example, the system 400 can include an actuation system 402 (which can be implemented with similar features as the actuation system 152 of the system 100, such as including a hammer). The actuation system 402 can include actuators 404, 406, where the actuator 404 can drive translation (e.g., horizontal linear translation, such as relative to the view in FIG. 17A) of a hammer 408 (e.g., a spring-loaded hammer, such as the hammer 192 shown in FIGS. 12A and 12B) while the actuator 406 can drive translation (e.g., vertical linear translation, such as relative to the view in FIG. 17A) of a plunger 410. As shown in FIG. 17A, the plunger 410 can extend across the entire plurality of hollow tubes 412, including extending across each segment of hollow tubes 412. Although each segment 414, 416 of the plurality of hollow tubes 412 (e.g., each of which include a plurality of hollow tubes) includes six segments in the non-limiting example of FIG. 17A; in other configurations, the plurality of hollow tubes 412 can include other numbers of segments.

The actuation system 402 can include springs 418, 420, which can be biased or otherwise store mechanical energy, which can be quickly released to provide the relatively high impulse force needed to puncture skin tissue (e.g., the epidermis). As described below, these springs 418, 420 can be unloaded and loaded cyclically, such that for each cycle, the springs 418, 420 can insert a single segment of the plurality of hollow tubes 412 (e.g., the segment 414). In this way, the springs 418, 420 can, when released, provide a relatively high impulse force needed, for a relatively small number of hollow tubes (e.g., as opposed to the entire plurality of hollow tubes 412, such as the entire array of tubes). In other words, the springs 418, 420 may not be able to store enough mechanical energy needed to drive all the segments of the plurality of hollow tubes 412 at the same time (e.g., simultaneously). Further, this configuration, can allow the springs 418, 420 and the actuator 406 to be made much smaller (than say a large solenoid), which can greatly minimize the spatial footprint of the device. In some configurations, however, the system 400 can drive all the plurality of hollow tubes 412 simultaneously into the tissue 422 (e.g., described as skin tissue). For example, the hammer 408, and more specifically the driver of the hammer 408, can extend across a large portion of the plurality of hollow tubes 412 (e.g., multiple segments, or all the plurality of hollow tubes) to simultaneously drive the large portion of hollow tubes into the tissue 422. Correspondingly, rather than including the actuator 406, the system 400 can include a brake (e.g., an electrically actuated brake, a mechanically actuated brake, etc.) that, when engaged with the plunger 410, prevents the springs 418, 420 from unloading. Then, when this brake is released, the plunger 410, via the unloading of the springs 418, 420, can drive the large portion of the plurality of hollow tubes 412 into the tissue 422. In some cases, the springs 418, 420 can be loaded and subsequently locked by the brake at a manufacturing plant, factory, etc., to advantageously provide a much smaller system 400. In this case, then, the system 400 need not include the actuator 404 (e.g., the move the hammer 408), and rather the hammer 408 can be coupled to a component of the system 400 (e.g., a housing 424 of the system 400, which can be implemented in a similar way to the housing 174).

As shown in FIG. 17A, each spring 418, 420 can be coupled to the plunger 410 and the housing 424. More specifically, the spring 418 can be coupled between the plunger 410 and a mount 426 that is also coupled to the housing 424 and in fixed relationship to the housing 424. Correspondingly, the spring 420 can be coupled between the plunger and the mount 426. Although two springs 418, 420 are shown in FIG. 17A, with the springs 418, 420 being positioned to the side of the actuator 406, in other configurations, these components can be situated differently. For example, the actuator 406, which can be coupled to the housing 424, can be positioned between the springs 418, 420. Further, although two springs 418, 420 are shown, which can be advantageous to better distribute the force along the length of the plunger 410, the system 400 can include other numbers of springs (e.g., one, three, four, etc.). For example, the system 400 can include a spring (e.g., a single spring) and can be coaxial with the actuator 406 (e.g., a portion of the actuator 406, such as a lead screw). In this way, the system 400 can be less bulky and the spatial footprint can be minimized. Correspondingly, although the actuator 406 is positioned to the side of the springs 418, 420 so as to better illustrate the individual components, the actuator 406 can be positioned in the middle of the plurality of hollow tubes. For example, the actuator 406 can bisect the plurality of hollow tubes, can be positioned such that at least one segment is positioned on opposing sides of the actuator 406, etc. In this way, the actuator 406 can better distribute forces to the plunger 410 (e.g., avoiding undesirable rotation of the plunger 410).

In some non-limiting examples, the system 400 can include a brake 428 to prevent or otherwise block unloading of the springs 418, 420 and therefore block movement of the plurality of hollow tubes 412 into the tissue 422 (e.g., block translation, actuation, etc., of a number of the plurality of hollow tubes 412). As shown in FIG. 17A, the actuator 406 can include the brake 428 (e.g., the brake 428 can be integrated or otherwise coupled to the actuator 406). For example, the brake 428 can engage and disengage with a lead screw of the actuator 406. When the brake 428 is engaged with the lead screw and locks, the lead screw is prevented from rotating, which blocks translation of the plunger 410 and correspondingly blocking unloading of the springs 418, 420. When the brake 428 is disengaged with the lead screw and unlocks, the lead screw is allowed to rotate, thereby allowing the plunger 410 to translated and correspondingly allowing unloading of the springs 418, 420. In some cases, the brake 428 can be electrically or mechanically actuated. For example, in the electrically actuated case, a computing device (e.g., of the system 400) can cause the brake to unlock and lock, as desired. In the mechanically stored case, a user can press a button to unlock the brake 428. Although the brake 428 is shown integrated within the actuator 406, which can be advantageous from a spatial footprint perspective (including when a spring is integrated with the actuator 406, such as in the coaxial configuration), the brake 428 can be positioned outside the actuator 406. For example, the brake 428 can be coupled to the housing 424 and can engage and disengage with the plunger 410. In some cases, the brake 428 can include a motor that drives and retracts a block to make contact with the plunger 410 (e.g., to lock the plunger 410) and to retract to cease contact with the plunger 410 (e.g., to unlock the plunger 410). As another example, the brake 428 can be implemented as an actuator (e.g., a linear actuator that extends and retracts). Regardless of the configuration, the brake 428 can facilitate controlled unloading of the springs 418, 420 (or spring, such as when implemented with a single spring).

In some non-limiting examples, the actuator 406, which can be coupled to the plunger 410 (e.g., the plunger 410 being coupled to a free end of the actuator 406) and can drive unloading and loading of the springs 418, 420 (e.g., each of which can be a compression spring), and thus can drive movement of the plunger 410 (e.g., upward translational movement of the plunger 410, and which can include avoiding rotational movement of the plunger 410). For example, with the brake 428 unlocked, the actuator 406 can be moved (e.g., retracted, such as upwardly) to load or otherwise bias the springs 418, 420 such that the plunger 410 is in a loaded position (i.e., shown in FIG. 17A). Then, the system 400 (e.g., the actuator 406) can cause the brake 428 to lock the plunger in position. In some non-limiting examples, the loaded position permits a clearance (e.g., a gap) between the plunger 410 and the plurality of hollow tubes 412. In this way, the actuator 404 can move the hammer 408 to the desired position (e.g., align the hammer 408 with the desired segment within the plurality of hollow tubes 412) even when the springs 418, 420 are loaded. In other configurations, however, such a clearance is removed, with the plunger 410 being in contact with the hammer 408 (e.g., only slightly so as not to advance the segment). In this case, the actuator 406 can be moved (e.g., retracted) until such a clearance exists prior to locking the brake 428.

FIG. 17A shows the system 400 with the springs 418, 420 loaded and with the plunger 410 in the loaded position just prior to releasing of the brake 428 and insertion of the segment 414 into the tissue 422. For example, the hammer 408 is aligned with the segment 414 and the plunger 410 is above on top of hammer 408 (e.g., with the hammer 408 being positioned above the segment 414). As shown in FIG. 17A, in the loaded position, none of the plurality of hollow tubes 412 are in contact with the tissue 422 (e.g., but as described below, a housing, such as the peripheral housing of the cartridge of the plurality of hollow tubes 412 can be in contact with the tissue 422).

FIGS. 17B-17C show the remaining steps of a cycle of unloading and loading of the springs 418, 420 to drive actuation of individual segments into the tissue 422. For example, after the brake 428 is released (e.g., by using a computing device to, for example, unlock the plunger 410, actuator 406, etc.), the springs 418, 420 unload to drive the plunger 410 into the hammer 408, which drives movement of the segment 414 into the tissue 422. More specifically, after the brake 428 is released, the plunger 410 translates downwardly via unloading of the springs 418, 420, thereby translating the plunger 410 downwardly, which also translates the segment 414 downwardly into the tissue 422. FIG. 17B shows this configuration where the springs 418, 420 are unloaded with the plunger 410 in an unloaded position and with the segment 414 inserted into the tissue 422 (e.g., each hollow tube of the segment 414 inserted into the tissue 422). At this point, the actuator 406 translates the plunger 410 upwardly to load the springs 418, 420 again (e.g., to reload the springs 418, 420). As the plunger 410 moves upwardly, the spring of the hammer 408 causes the hammer 408 to retract upwardly (e.g., the drive of the hammer 408) until the hammer 408 reaches a home or default position. Then, the plunger 410 can be further translated upwardly until the plunger 410 does not contact the hammer 408, and then the brake 428 can be engaged to lock the plunger 410. At this point, the actuator 404 can move (e.g., translate horizontally) the hammer 408 until the hammer 408 is aligned with a desired segment, such as the segment 416. In some cases, the actuator 406 can lower the plunger 410 until the plunger 410 contacts the hammer 408 (e.g., but with the segment not being translated). The reloading position is shown in FIG. 17C with the plunger 410 in a loaded position, with the springs 418, 420 loaded, and with the hammer 408 aligned with the segment 416. The FIGS. 17A-17C therefore detail a single cycle of unloading and loading the system 400. This cycle can be repeated a number of times, until, for example, all the segments of the hollow tubes 412 are inserted into the tissue 422.

Although the hammer 408 is shown as only being aligned with a single segment of the plurality of hollow tubes 412, in other configurations, the hammer 408 can extend across multiple segments (e.g., the hammer 408 can be made bigger, such as wider). In this case, the hammer 408 can drive multiple segments (e.g., the segments 414, 416 together) simultaneously into the tissue 422. In this way, the system 400 can more quickly insert the plurality of hollow tubes 412 into the tissue 422.

As described above, the system 400 can advantageously individually insert particular segments of the plurality of hollow tubes 412 (e.g., an array of hollow tubes), which requires less insertion forces and force impulses than inserting the entire array. Further, since the springs 418, 420 (or a single spring) can drive insertion of an individual segment into the tissue, the actuator 406 (e.g., a vertical actuator to drive vertical translation of the plunger 410, so as to load the springs 418, 420) can be made much smaller and can be implemented as a less power intensive actuator. For example, a solenoid can be used to directly drive individual segments of the array into tissue. However, solenoids can be power intensive, and in particular require large influxes of current. Therefore, the power source used to drive the solenoid is typically a cord (e.g., connected to an AC power source). Although the solenoid can quickly advance each segment being directly connected to an AC power source, in some configurations, the cord and the relatively large size of the solenoid can be burdensome. In this configuration, however, since the actuator 406 can be advanced slowly to slowly load the springs 418, 420, the power requirements (e.g., the current requirements) can be significantly decreased. Accordingly, the actuator 406 can be implemented as a DC motor that has lower power requirements as opposed to an AC motor, and the actuator 406 can be made smaller than a solenoid. Further, with these lower power requirements, as described below, the power source of the system 400 can be cordless (e.g., a battery pack) which can provide the requisite power requirements to the system 400.

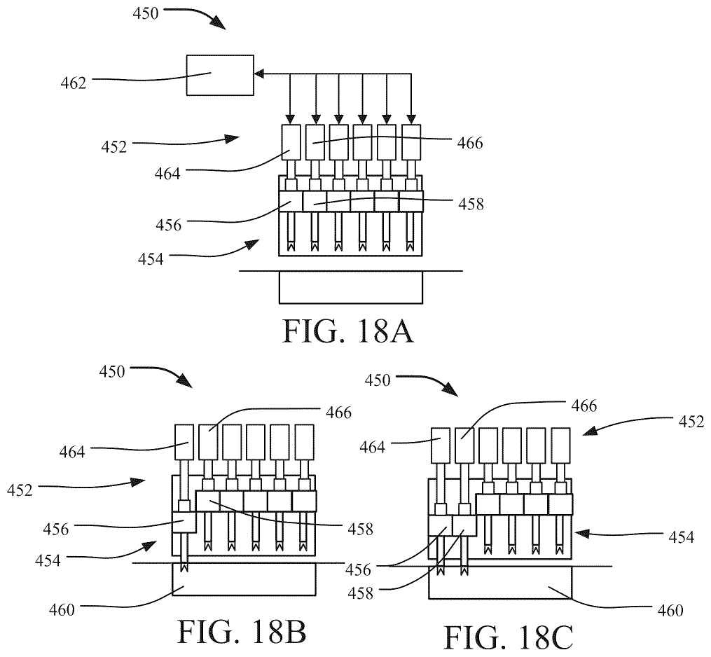

FIGS. 18A-C show an example of a system 450, which can be a specific implementation of other systems described herein. The system 450 can include an actuator system 452 which can cause a plurality of hollow tubes 454, which include segments 456, 458 of hollow tubes to be inserted into a tissue 460 (e.g., skin tissue). The system 450 can include a computing device 462, which can control some or all of the components of the system 450. The actuator system 452 can include a plurality of actuators, with each actuator being configured to translate one or more segments of the hollow tubes 454 into the tissue 460. For example, the actuator system 452 can include actuators 464, 466. Although the actuator system 452 is described as having actuators 464, 466 for simplicity, in FIG. 18A, the actuator system 452 includes an actuator for each respective segment. Therefore, FIG. 18A shows the actuator system 452 having six actuators. Correspondingly, although the system 450 is described as having segments 456, 458 for simplicity, in FIG. 18, the system 450 can include more segments, and is illustrated as having six segments, with each segment corresponding with a respective actuator. Further, although each actuator is shown as driving an individual segment of the plurality of hollow tubes 454, in other configurations, the actuator can drive multiple segments (e.g., for faster insertion of the hollow tubes 454 into the tissue 460). For example, each actuator can drive at least two segments of the hollow tubes 454 into the tissue 460. In some configurations, each actuator driving a respective individual segment only (e.g., only driving one segment) can be advantageous in that the actuators can be made much smaller.

The actuators can be implemented in different ways. For example, each actuator can be a linear actuator, such as a solenoid. As another example, each actuator can be a spring and a brake, or can include a spring and a brake (e.g., implemented in a similar manner as the configuration in FIG. 17A-17C). In this way, the spring can drive downward translation of one or more of the corresponding segments of the hollow tubes 454. As yet another example, each actuator can be a pneumatic actuator (e.g., driven by gas, such as air, by, for example, opening a valve to drive air into the pneumatic actuator and cause the pneumatic actuator to insert the corresponding segment into the tissue 460). As shown in FIG. 18A, the computing device 462 can be in communication (e.g., bidirectional communication) with each of the actuators (e.g., the actuators 464, 466). In this way, the computing device 462 can cause selective activation of a particular actuator and therefore corresponding translation of a corresponding segment of the hollow tubes 454 into the tissue 460. For example, when the actuator is a linear actuator (e.g., a solenoid), the computing device can cause a motor of the actuator to advance the actuator (e.g., by rotating a lead screw of the actuator). As another example, when the actuator includes a spring and a brake, the computing device can cause the brake to release thereby allowing the spring to unload (e.g., to advance a segment). As yet another example, when the actuator is a pneumatic actuator, the computing device can cause a valve to open to permit fluid (e.g., gas, such as air) into a piston to drive the piston (e.g., to advance the segment into the tissue 460).

FIG. 18A shows the system 450 prior to extension of any of the actuators and prior to translation of any of the segments of the plurality of hollow tubes 454. FIG. 18B shows the system 450 after the actuator 464 has been extended (e.g., by the computing device 462) and the segment 456 is translated downward into the tissue 460 (e.g., by the actuator 464 contacting and driving the substrate of the segment 456 and the hollow tubes coupled thereto into the tissue 460). FIG. 18C shows the system 450 after the actuator 458 has been extended (e.g., by the computing device 462) and the segment 458 is translated downward into the tissue 460 (e.g., by the actuator 466 contacting and driving the substrate of the segment 458 and the hollow tubes coupled thereto into the tissue 460).

In some non-limiting examples, the actuation system 452 can be advantageous in that rather than having a single vertical actuator drive vertical translation of multiple segments (e.g., all the segments) of the plurality of hollow tubes, which requires a relatively large actuator (e.g., solenoid), a plurality of actuators can be made smaller and can take up a smaller footprint. Similarly, with a plurality of smaller actuators, a horizontal actuator (e.g., to position the hammer at a specific location) can be removed. Accordingly, in some cases, the system 450 does not include any horizontal actuators, hammers, etc.

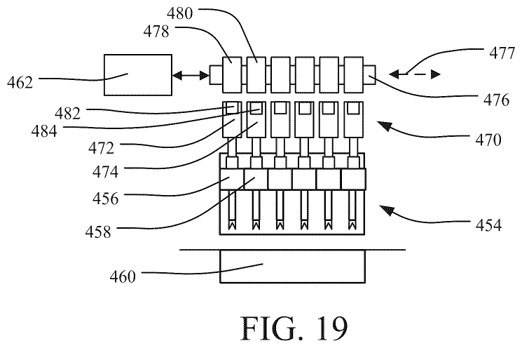

In some non-limiting examples, in which each of the actuators are implemented as pneumatic actuators, rather than electrically opening valves to allow driving fluid in (e.g., air) to drive the respective actuator and thus the corresponding segment into the tissue, each actuator can include a valve that interfaces with a cam of a camshaft to selectively drive different segments into the tissue. FIG. 19 shows an example of an actuation system 470 that includes these features. For example, the actuation system 470 can include a plurality of actuators that include actuators 472, 474, a camshaft 476 that includes a plurality of cams including cams 478, 480, and a plurality of valves including valves 482, 484, and a pneumatic source (e.g., a pressure source, a pneumatic manifold, etc.). Although only two actuators, two cams, and two valves are described for simplicity, it is appreciated that the actuation system 470 can include other numbers of these components, such as illustrated, in which the actuation system 470 includes six of each of these components. As shown in FIG. 19, each valve 482, 484 is coupled to an integrated within a respective actuator 472, 474, however, the valves 482, 484 need not be coupled to or integrated with a respective actuator, and can, for example, be positioned to a side of the actuators 472, 474. Each valve 482, 484 can be fluidly coupled to a respective actuator 472, 474. In some cases, each valve 482, 484 is only fluidly coupled to the respective actuator 472, 474, to, for example, only cause fluid to enter the respective actuator 472, 474. The camshaft 476 can selectively cause each actuator 472, 474 to extend, driving the corresponding one or more segments of the plurality of hollow tubes 454 into the tissue 460. More specifically, the camshaft 476 can rotate around an axis 477, by for example, a motor coupled to the camshaft 476 and in communication with the computing device 462. As the camshaft 476 rotates in a first direction, the cam 478 comes into contact with the valve 482 to open the valve 482 and cause fluid to enter the actuator 472 to drive the segment 456 into the tissue 460. At this point, only the cam 478 contacts the valve 482, with the other cams not being in contact with the other valves (e.g., or otherwise opening the other valves). In this way, the camshaft 476 can selectively cause only some (e.g., one) of the actuators to extend and drive the respective segment (or segments) into the tissue 460. As the camshaft 476 rotates further in the first direction, the cam 480 comes into contact with the valve 484 to open the valve 484 and cause fluid to enter the actuator 474 to drive the segment 458 into the tissue 460 (e.g., in a similar manner as the actuator 472). This process can proceed until each segment of the plurality of hollow tubes 454 is inserted into the tissue 460.

Although one cam can cause one valve to open, thereby causing one actuator to extend, in other configurations, one cam can cause multiple valves to open (e.g., to cause multiple actuators to extend), or valve can be fluidly coupled to multiple actuators (e.g., such that when the valve is opened, the actuators extend).

Although the actuation system 470 has been described as including pneumatic actuators, in other configurations, the actuation system 470 can include one or more actuators that are spring-loaded (e.g., each actuator of the actuation system 470 including the actuators 472, 474 are spring-loaded actuators). In this case, each actuator 472, 474 includes a piston and a spring to drive the piston, and the camshaft 476 can both load the springs and subsequently release the springs to drive the piston and thereby drive the segment into the tissue 460. For example, each cam 478, 480 can interface with the piston of each respective actuator 472, 474 (or a component thereof). As the camshaft 476 rotates in a first rotational direction (e.g., clockwise), the cam 478 interfaces with the actuator 472 to load the spring (e.g., by lifting the piston upwards). Further rotation of the camshaft 476 in the first rotational direction can release the engagement between the cam 478 and the actuator 472 (e.g., the cam 478 unblocks the actuator 472) and the spring is unloaded to drive the piston of the actuator 472 into the segment 456 and thereby drive the hollow tubes of the segment 456 into the tissue 460. In some cases, the further rotation of the camshaft 476 in the first rotational direction can cause the cam 480 to load the spring of the actuator 474 in a similar manner as the cam 478 and the actuator 472. Therefore, advantageously, the camshaft 476 can simultaneously release or unblock one actuator (e.g., the actuator 472) while at the same time loading the spring of another actuator (e.g., the actuator 474). In this way, the overall speed of the insertion of all the segments of the plurality of hollow tubes 454 can be faster. However, in other configurations, it can be desirable to load a given actuator only after an adjacent actuator (or other actuator) has been unloaded (e.g., for better control of the insertion) and thus the camshaft 476 can be configured to load the actuators one at a time (e.g., by orienting the cams). Similarly to the pneumatic configuration, each cam of the camshaft 476 can load multiple actuators and each actuator can drive multiple segments into the tissue 460 at the same time.

In some configurations, the actuation system 470 can include a motor (e.g., an electric motor) to drive the rotation of the camshaft 476. Further, the computing device 462 can cause the motor to rotate the camshaft 476. In some configurations, the motor having fine angular movements, such as the motor being a stepper motor, can be advantageous to position the camshaft 476 and the cams of the camshaft 476 appropriately.

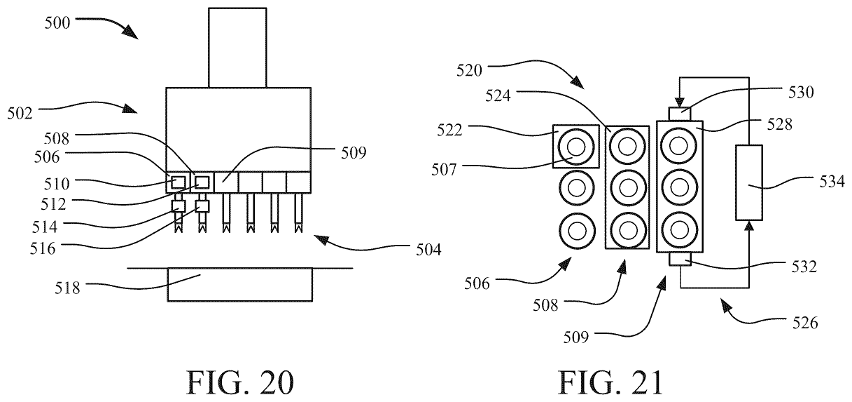

FIG. 20 shows an example of a system 500 (e.g., a skin grafting system), which can be a specific implementation of other systems described herein. For example, the system 500 can include an actuation system 502 (which can be implemented with similar features as the actuation system 152 of the system 100, such as including a hammer). The system 500 can include a plurality of hollow tubes 504 having multiple segments (e.g., segments 506, 508 of the plurality of hollow tubes 504). The actuation system 502 can include one or more electroacoustic transducers (e.g., a piezoelectric transducer) that, when powered, causes one or more hollow tubes of the system 500 to vibrate. As shown in FIG. 20, the actuation system 502 can include electroacoustic transducers 510, 512, 514, 516. In some cases, the electroacoustic transducers 510, 512 can be coupled to respective segments 506, 508 (e.g., the substate of the segment). In this way, when a respective electroacoustic transducer is powered, the electroacoustic transducer only vibrates the segment including the hollow tubes coupled thereto. In some cases, and as illustrated, the electroacoustic transducers 514, 516 can be coupled to a hollow tube (e.g., a single hollow tube) of the respective segment 506, 508. In this way, the vibration provided by the electroacoustic transducer is better targeted to the specific hollow tube and vibrational losses can be mitigated.

In some cases, the electroacoustic transducers can be powered to cause one or more of the hollow tubes to vibrate during insertion (e.g., translation) of the one or more hollow tubes into a tissue 518 (e.g., skin tissue), such as by an actuator, or after the insertion has been completed. In some configurations, applying vibration during insertion can help sever the portion (or micrograft) from the surrounding tissue, particularly when the hollow tube is not rotated around its axis to cut the tissue. Further, applying vibration after insertion can help remove the bottom of the portion (or micrograft) from the surrounding tissue. In some configurations, having an electroacoustic transducer on each segment can help target vibration only to the hollow tubes in that segment (e.g., during insertion or after insertion), while avoiding unwanted vibration at other segments. For example, during insertion (or after insertion, such as complete insertion) of the segment 506, the electroacoustic transducer 510 can be powered to cause vibration of the hollow tubes of the segment 506. Correspondingly, the other remaining electroacoustic transducers can be refrained from being powered, so as to not only avoid undesirable vibration while tubes are not inserted, but also conserve power (e.g., by not powering the electroacoustic transducers).

In some non-limiting examples, each electroacoustic transducer (e.g., the electroacoustic transducers 510, 512, 514, 516) can vibrate the corresponding one or more hollow tubes at an ultrasonic frequency (e.g., at a frequency that is substantially greater than or equal to 15 kHz, 20 kHz, etc.). This relatively fast vibration, when applied to a hollow tube, can better sever tissue, with the hollow tube acting as a saw through the tissue. In some cases, including when a hollow tube is better suited for solely translational insertion and severing of the tissue (e.g., when the hollow tubes include at least two or only two extensions at a distal end thereof), the electroacoustic transducers 510, 512, 514, 516 can vibrate substantially along a longitudinal axis of the hollow tubes (e.g., where each longitudinal axis of each hollow tube is substantially parallel to each other). In this way, the hollow tubes can better insert into the tissue since the vibration is applied to align with the cutting edge of the hollow tube, rather than vibration wasted to side to side that may not actually cut or sever the tissue (and can simply destabilize the hollow tube). In other cases, including when the hollow tubes are configured to rotate around an axis thereof (e.g., the hollow tube 180) with each having a cutting edge that curves around an axis of the respective hollow tube, the electroacoustic transducers 510, 512, 514, 516 can vibrate substantially angularly, or in other words, vibrate substantially circularly so as to again, align the vibration with the curved cutting edge of the hollow tube. Although the system 500 has been described as vibrating the hollow tubes at ultrasonic frequencies, the system 500 (e.g., the electroacoustic transducers 510, 512, 514, 516) can vibrate specific hollow tubes at non-ultrasonic frequencies, such as frequencies less than those in the ultrasonic range. Further, although the system 500 has been described as causing vibration with electroacoustic transducers 510, 512, 514, 516, in other configurations, the system 500 can vibrate the hollow tubes in other ways, such as, for example, using a motor (e.g., and a pulley rotationally coupled to the motor, such as the rotator described previously).

In some configurations, when vibrating at ultrasonic frequencies, the relatively fast vibrations can undesirably overheat the harvested tissue (e.g., the micrograft captured in the respective hollow tube), leading to, among other things, death of the harvested tissue. Therefore, in some cases, the system 500 can include a cooling system 520 to cool or otherwise remove heat from one or more of the hollow tubes (e.g., during or after powering of an electroacoustic transducer vibrating at an ultrasonic frequency).

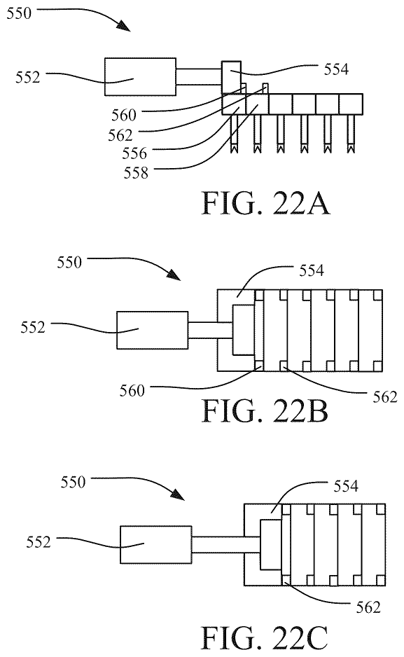

The cooling system 520 can be implemented in different ways. For example, the cooling system 520 can include a heat sink 522 that can be coupled to a hollow tube 507 of the segment 506. The heat sink 522 can remove heat from the hollow tube 507 and transfer the heat to the ambient environment (e.g., via one or more cooling fins). In some cases, the heat sink 522 can be coupled to a proximal end of the hollow tube 507, opposite to its distal end that can include one or more extensions that are inserted into the tissue 518. In this way, the heat sink 522 is avoided from interacting with the tissue 518. In other cases, the heat sink 522 can be coupled to the hollow tube 507 away from its proximal end. In this way, heat is removed closer to where the heat removal is needed (e.g., where the harvested tissue is retained). The cooling system 520 can include a heat sink 524, which can be coupled to multiple or all of the hollow tubes of the segment 508. The heat sink 524 can be implemented in a similar way to the heat sink 522. In some cases, the cooling system 520 can include a heat transfer system 526 that can remove heat from one or more hollow tubes of a segment 509 of the plurality of hollow tubes 504. The heat transfer system 526 can include a chamber 528, an inlet 530 fluidly coupled to the chamber 528, an outlet 532 fluidly coupled to the chamber 528, and a heat exchanger 534. The chamber 528 can surround one or more hollow tubes of the segment 509 and can function as a heat exchanger that removes heat form the hollow tubes. As shown in FIG. 21, a refrigerant (e.g., water, gas, etc.) can be directed (e.g., pumped) through the inlet 530, through the chamber 528, and out the outlet 532. The refrigerant can absorb heat from the hollow tubes within the chamber and can pass to the heat exchanger 534 to remove heat from the refrigerant (e.g., by directing the heat into the ambient environment). In this way, the heat transfer system 526 can actively cool down the hollow tubes (e.g., rather than passively, such as with a heat sink). Although FIG. 22 shows the heat transfer system 526 being applied to a segment of the hollow tubes 504, in other configurations, the heat transfer system 526 can be applied to multiple segments (e.g., all segments) of the hollow tubes 504.

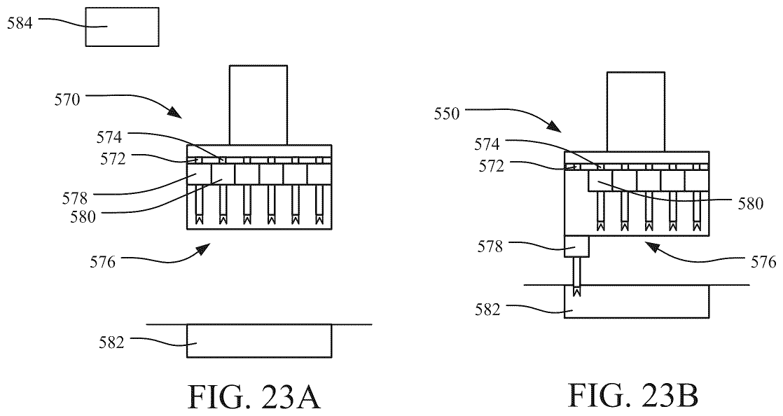

FIG. 22A-C show an example of a detection system 550, which can be implemented with and using other devices and components herein. For example, the detection system 550 can include an actuator 552 (e.g., a horizontal actuator), which can be implemented in a similar way to the actuator 156. The actuator 552 can include a hammer 554 used to drive insertion of segments 556, 558 of hollow tubes (e.g., of a plurality of hollow tubes) into tissue. As shown in FIG. 23A, each segment 556, 558 can include a respective stop 560, 562 (e.g., coupled to and extending from a respective substrate of the segment 556, 558). Although each stop 560, 562 is shown as being a block (e.g., a rectangular prism), each stop 560, 562 can have other shapes. The actuator 552 can detect that a particular segment has been inserted into tissue. For example, the actuator 552 can advance the hammer 554 until the hammer 554 contacts the stop 560 (e.g., which is indicated by an increase in current draw of the actuator 552 due to increased resistance between the hammer 554 and the stop 560, and more specifically the motor of the actuator 552). In some cases, at this point, the actuator 552 can retreat the hammer 554 and can subsequently advance the hammer 554 until the hammer 554 contacts the stop 560. In this way, it can be ensured that the hammer 554 is actually contacting the stop 560. Then, the segment 556 can be inserted, as described above. Once the segment 556 is inserted, the stop 560 is positioned below the hammer 554, such that the hammer 554 is free to move past the stop 560. FIG. 22B shows a top view of the detection system 550, in which the hammer 554 contacts the stop 560. At this point, the actuator 552 can advance the hammer 554 until the hammer 554 contacts the stop 562 (e.g., in a similar way to the hammer 554 contacting the stop 560). In this case, since the actuator 552 has detected contact with the stop 560 (e.g., by an increase in current draw to the actuator 552) previously, and has indicated contact with the stop 562, the detection system 550 (e.g., a computing device of the detection system 550) can determine that the segment 556 has been inserted and that the segment 558 is to be inserted. FIG. 22C shows a top view of the detection system 550, in which the hammer 554 contacts the stop 562.

Although one stop has been described as being coupled to a respective segment (e.g., a substate of a segment), in other configurations, and as illustrated in FIGS. 22B and 22C, each segment can include multiple stops (e.g., two stops). For example, a first stop can be positioned on one end of the segment and a second stop can be positioned at an opposing end of the segment. In this way, the hammer 554 can contact both stops of the segments at the same time, which can avoid issues with offsetting of the hammer 554 (e.g., the hammer 554 not being completely aligned with a given segment). Although each segment is shown as having a stop, in other configurations, segments can lack stops, such that the detection of a number of segments having been inserted is detected at intervals that include multiple segments. For example, a stop can be positioned after two segments, three segments, four segments, five segments, six segments, etc. In some non-limiting examples, the detection system 550 can be advantageous in that other additional sensors, such as, at each segment is not needed. This can advantageously decrease complexity of the broader system.

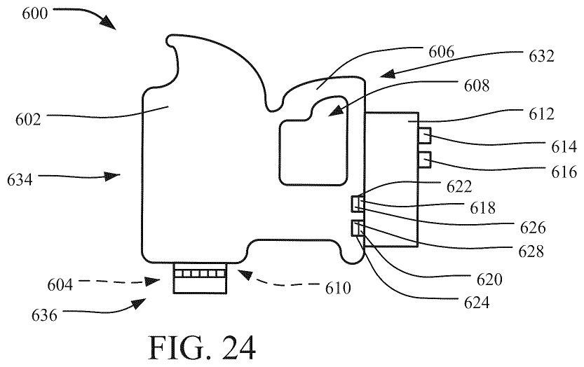

FIGS. 23A and 23B show an example of a detection system 570, which can be a different implementation than the detection system 570 (or can be used together with the detection system 550 for redundancy). The detection system 570 can include a plurality of sensors that include sensors 572, 574. Each sensor 572, 574 can detect a respective segment 578, 580 of a plurality of hollow tubes 576. Although there are only two sensors 572, 574 described along with respective segments 578, 580 for simplicity, the system can include other numbers of sensors, segments, etc., such as, for example, three, four, five, etc. As a specific example, and as shown in FIG. 24A, the plurality of hollow tubes 576 include six segments, and the detection system 570 can include six sensors.

Each sensor 572, 574 can detect when a particular segment is moved (e.g., translated downwardly) into the tissue 582 (e.g., skin tissue). For example, prior to actuation (e.g., translation of the segment 578), the sensor 572 can sense that the segment 578 has not been actuated (e.g., translated downwardly), by, for example, a computing device receiving sensor data from the sensor 572 (e.g., the sensor data being indicative of a value that exceeds a magnitude threshold). Then, after the segment 578 has been actuated by the actuation system, the sensor 572 can sense that segment 578 has been actuated and the one or more hollow tubes of the segment 578 are inserted into the tissue 582, again, by for example, the computing device receiving sensor data from the sensor 572 (e.g., the sensor data being indicative of a decrease in a magnitude of a signal). FIG. 23B shows the segment 578 actuated and inserted into the tissue 582 with the segment 580 having not been actuated. The sensor 574 can sense the segment 580 in a similar way to the sensor 572 sensing the segment 578.

In some non-limiting examples, a computing device 584, which can be in communication (e.g., bi-directional communication) with some or all of the components of the system (e.g., a skin grafting system) including the detection system 570, can check that each segment (or one or more segments) has been inserted into the tissue 582 before actuating or otherwise inserting the next segment into the tissue. For example, the computing device 584 can receive, from the sensor 572, sensor data indicative of the segment 578 in a non-actuated position (e.g., a first position). Then, the computing device 584 can cause the actuation segment to insert the segment 578 into the tissue 582. At this point, the computing device 584 can receive, from the sensor 572, sensor data indicative of the segment 578 being in an actuated position (e.g., a second position, in which the segment 578 is inserted into the tissue 582). If at this point, the computing device 584 receives sensor information indicative of the non-actuated position, the computing device 584 can notify a user that an error has occurred (e.g., by causing a display to be presented to a user, causing a light to flash, e.g., red or orange, etc.). Further, the computing device 584 can lock the system, and more specifically, the actuation system, to prevent any additional segments or hollow tubes from being subsequently inserted into the tissue 582. In this way, the computing device can ensure that the system is functioning properly before inserting additional hollow tubes. If the computing device 584 receives sensor information indicative of the segment 578 being in the actuated position, the computing device 584 can then proceed to receive sensor information from the sensor 574, or can cause the actuation system to insert the segment 580 into the tissue 582. This process can proceed until all the segments of the plurality of hollow tubes 576 have been inserted. In other cases, however, similarly to the detection system 450, the detection system 570, rather than checking each segment and having a corresponding sensor for each segment, the detection system 470 can include fewer numbers of sensors to detect a segment after a number of segments greater than one have been inserted. For example, a sensor can be positioned at every second segment, every third segment, every fifth segment, every sixth segment, and so on. As described above, this can decrease electrical complexity and can further speed up the insertion procedure (e.g., by saving computation time from checking each sensor).

The sensors 572, 574 can be implemented in different ways. For example, each sensor can be implemented as a microswitch. In this case, a portion of the switch can be coupled to the segment (e.g., the substrate of the segment). The portion can be electrically coupled to the remaining portion of the switch (e.g., coupled to the housing) when the segment is in a non-actuated position. Then, when the actuation system advances a segment, the portion can be decoupled from the remaining portion of the switch. In this way, when electrically coupled, the presence of a voltage signal (e.g., 5V) can indicate that the segment is in a non-actuated position and when electrically decoupled, the lack of a voltage signal (e.g., 0V) can indicate that the segment is in an actuated position. As another example, each sensor can be implemented as an optical sensor (e.g., a photoresistor). In this case, the absence of a light signal (e.g., from the ambient environment) can indicate that the segment is in a non-actuated position (e.g., by the substrate of the segment blocking light from being received by the optical sensor). Conversely, the presence of a light signal (e.g., from the ambient environment, in which the substrate of the segment is moved out of alignment with the optical sensor), can indicate that the segment is in an actuated position (e.g., by the substrate of the segment allowing light to the optical sensor). In some configurations, the optical sensor can be a laser, which can sense the distance the segment has traveled (e.g., where the optical sensor can be a time of flight sensor). As yet another example, each sensor can be a Hall effect sensor. In this case, each segment can include a magnet (e.g., a magnet can be coupled to a substrate of each segment). In this case, in the non-actuated position, the magnet is in close proximity to the sensor and can cause a switch of the sensor to be closed (e.g., due to the magnetic flux). Conversely, in the actuated position, in which the segment and magnet are positioned away from the sensor, a switch of the sensor can be opened (e.g., due to lack of a sufficient amount of magnetic flux).

In some non-limiting examples, some or all of the components of the detection system 570 can be implemented within a cartridge that includes the plurality of hollow tubes 576. For example, each sensor (e.g., the sensors 572, 574) can be coupled to and positioned within the cartridge. Further, the computing device 584 (or other computing device, such as a controller), can be coupled to and positioned within the cartridge. In this way, when the cartridge is disposed of, such as after use with an individual, the sensors, which in some cases must be in close proximity to the segments, can also be disposed of with the cartridge. This can prevent electrical routing to a different computing device that could provide pathways for ingress of bodily fluids, which could compromise the sterility of the housing of the system (e.g., a handled device in which the cartridge is removably coupled to).

In some non-limiting examples, the computing device 848 can determine that a harvesting procedure or sequence has been complete, such as, for example, after each segment is indicated, by a respective sensor (or the actuator 552) that the particular segment (or segments) have all been inserted into the tissue 582. In some cases, the computing device 848 can provide an alert, notification, etc., indicating that the harvesting procedure has been complete, based on the computing device 848 determining that the harvesting procedure has been complete. This alert, notification, etc., can be activating a light (e.g., flashing light), presenting a graphic on a display device, etc. In some cases, the computing device 848 can determine that the harvesting procedure or sequence has been complete after each segment has been retracted back to a starting position. For example, when each segment is driven into the tissue, each segment can be locked by a respective latch (e.g., to fix the position of each segment relative to the tissue 582) until each segment has been inserted into the tissue 582. Then, including after each segment has been inserted, each latch can be released and all the segments can be retracted. Once each segment is in a non-actuated position, the computing device 848 can determine that the harvesting procedure has been completed and can accordingly provide an alert, notification etc., accordingly. Therefore, in some cases, a computing device can determine that the harvesting procedure has been complete after each sensor has sensed the respective segment (or segments) has left the non-actuated position and has subsequently returned to the non-actuated position.

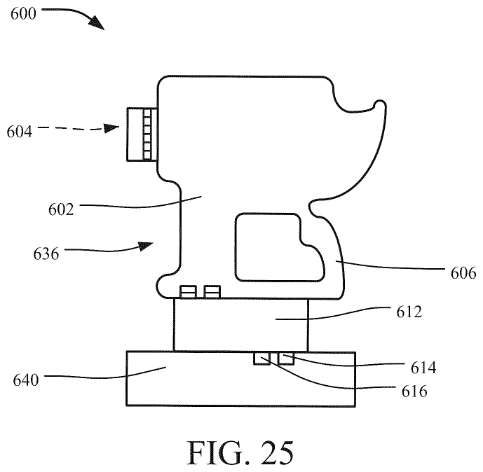

FIG. 24 shows a schematic illustration of an example of a system 600 (e.g., a skin grafting system), which can be a specific implementation of other system described herein. Therefore, the other systems described herein are applicable to the system 600 and vice versa. The system 600 can include a housing 602 and a plurality of hollow tubes 604 (e.g., implemented as a cartridge removably coupled to the housing 602). The housing 602 can be of a handheld device, such as the configuration illustrated in FIG. 24. The housing 602 can include a handle 606 and a slot 608 to receive a hand of user. As described in other configurations, the housing 602 can include an aperture 610 (e.g., a loading aperture, such as the loading aperture 1006) to receive the cartridge. In some configurations, the system 600 can include a battery pack 612 that can include one or more batteries (e.g., rechargeable batteries). The battery pack 612 can include one or more terminals that can interface with one or more electrical components of the system 600 (e.g., within the housing), or can inface with a charger. For example, the battery pack 612 can include terminals 614, 616, 618, 620 each of which can be electrically coupled to the batteries within the battery pack 612. Each terminal 614, 616 can be an input terminal, which can interface with a respective charging terminal of a charger so as to charge the batteries within the battery pack 612 (e.g., when the system 600 is not being used, such as during downtime). Each terminal 614, 616 can be electrically coupled to a particular type of electrode of the batteries. For example, the terminal 614 can be electrically coupled to an anode of each battery, while the terminal 616 can be electrically coupled to a cathode of each battery. Each terminal 618, 620 can be an output terminal, which can interface with a respective electrical port or contact of the system 600 (e.g., the electrical system of the system 600). For example, each terminal 618, 620 can be received within a respective recess 622, 624 of the housing 602 and can be electrically coupled to (and decoupled from) a respective electrical terminal 626, 628 (e.g., which can be a port, contact, etc.) that are coupled to the housing 602. Each electrical terminal 626, 628 can be electrically coupled to one or more electrical components of the system 600, such as for example, a power supply (e.g., to supply the power supply voltage and the negative or ground voltage). As shown in FIG. 25A, the electrical terminal 626 can be positioned within the recess 622, while the electrical terminal 628 can be positioned within the recess 624. However, in other configurations, the electrical terminals 626, 628 can extend from a surface of the housing 602. Similarly to the terminals 614, 616, the terminals 626, 628 can be electrically coupled to a particular type of electrode of the batteries. For example, the terminal 626 can be electrically coupled to each anode of each battery, while the terminal 628 can be electrically coupled to each cathode of each battery.

When the battery pack 612 is coupled to the housing 602, the battery pack 612, and more specifically the batteries therein, can provide power to the system 600 (e.g., one or more components of the system 600 including a motor, an actuator, an actuation system, a computing device, a display, a user interface, a light, a communication system, etc.). As described below, the batteries of the battery pack 612 can be rechargeable. For example, each battery can be a lithium ion battery, a lead acid battery, a Nickel-Cadmium battery, a Nickel metal Hydride battery, etc. In other configurations, the batteries of the battery pack 612 are non-rechargeable. In this case, the battery pack 612 can simply be discarded after use.

As shown in FIG. 24, the battery pack 612 can be coupled to a rear side 632 of the housing 602, which can be proximate to the handle 606. This can be advantageous in that the battery pack 612 does not obstruct the view during positioning of the cartridge on tissue (e.g., skin tissue). For example, when the battery pack is coupled to a front side 634 of the housing 602, the battery pack 612 extending away from the front side 634 (e.g., and away from the rear side 632) can obstruct the downward view of a user when placing the cartridge on tissue. As another example, when the battery pack 612 is coupled to the bottom side 636 of the housing 602 (e.g., which includes the cartridge and the corresponding aperture 610), the height of the battery pack 612 should be smaller than the height of the cartridge housing so as to ensure that the cartridge housing can contact the tissue.

In some non-limiting examples, the battery pack configuration (e.g., a cordless system) can be advantageous for a number of reasons. For example, with the battery pack 612, the system 600 can be much more mobile. In particular, a system 600 with a power cord could tether the system 600 and could require the power cord to be moved as the system 600 is moved. In some cases, however, when a system includes a power cord, the power cord can supply the requite current required for high current applications. For example, when the actuation system includes a solenoid as a vertical actuator to drive each segment into the tissue, the solenoid can insert each segment quickly, but the high speed requires larger amounts of current. In other configurations, when the actuation system is overall slower (e.g., the time required to insert all segments into tissue), the actuation system requires smaller current requirements and thus a battery pack, such as the battery pack 612, can be well suited for providing the requisite, lesser current requirements. Therefore, in some cases, the system 600 does not include a solenoid. More specifically, the system 600 does not include a solenoid that is an actuator (e.g., vertical actuator) to insert one or more hollow tubes into tissue. Correspondingly, when the system 600 includes the battery pack 612, the system 600 (e.g., a motor of an actuator) that inserts one or more hollow tubes into the tissue can be configured to still apply a driving force of greater than or equal to substantially 1.5, 2, 2.5 lbs. per hollow tube (e.g., for a given segment of the plurality of hollow tubes, such as ten hollow tubes) by using, for example, a reloadable spring. Similarly, when the system 600 includes the battery pack 612, the system 600 that inserts the one or more hollow tubes into the tissue can still be configured to drive the hollow tubes (e.g., a given segment of hollow tubes) into the tissue at a speed of greater than or equal to substantially 0.4, 0.5, 0.6, 0.7, 0.8 meters per second.

FIG. 25 shows a schematic illustration of the system 600, which includes a charger 640 engaged with the battery pack 612. The battery pack 612, when engaged with the charger 640, can receive power from the charger 640 (e.g., that is engaged with a standard power supply, such as 120V) to charge or recharge the batteries of the battery pack 612. As shown in FIG. 25, the housing 602 is coupled to a battery pack 612, which is coupled to the charger 640 (e.g., the battery pack 612 is positioned between the housing 602 and the charger 640). In this way, the housing 602 with the battery pack 612 coupled thereto can simply be docked with the charger 640 when the system 600 is not being used for procedures (e.g., during downtime where no procedure is being completed). In other configurations, the battery pack 612 can be decoupled from the housing 602 and can be engaged with the charger 640 to charge the batteries of the battery pack 612. In this way, the battery pack 612 can be recharged without the bulkiness of the housing 602.

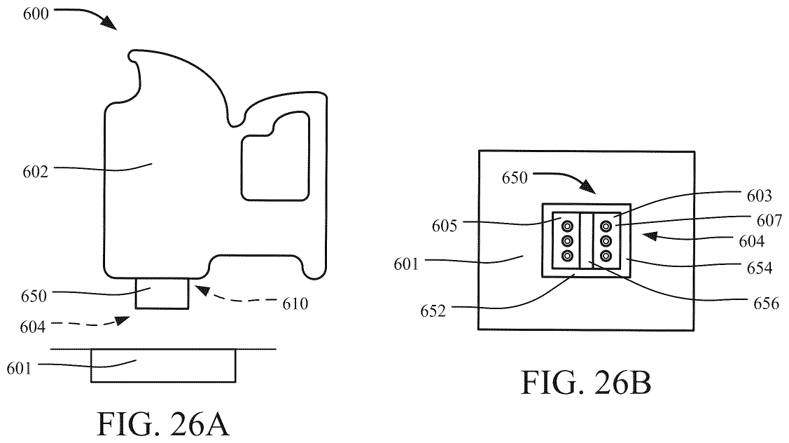

FIG. 26A shows a schematic illustration of the system 600 positioned above a tissue 601 prior to insertion of the plurality of hollow tubes 604 into the tissue 601. More specifically, FIG. 26A shows a specific configuration for stabilizing tissue during a skin grafting process. Typically during a skin grafting process, maintaining a constant, relatively high force or pressure on the tissue 601 helps during insertion of the hollow tubes into the tissue because the relatively high force applied to the tissue 601 tightens the tissue 601 or otherwise creates a taught surface that allows translational (or rotational) insertion of hollow tubes easier. Otherwise, without the high force or pressure, when a hollow tube is advanced, the tip of the hollow tube deflects the untaught surface of the tissue 601 without puncturing the tissue 601. As shown in FIG. 26A, the system 600 can include a cartridge 650 that can include and retain the plurality of hollow tubes 604 and can be removably coupled to the housing 602 at the aperture 610.

FIG. 26B shows a cross-sectional view of the cartridge 650 surrounded by the tissue 601. The cartridge 650 can include a cartridge housing 652 that surrounds the plurality of hollow tubes 604. More specifically, a peripheral edge 654 can surround the plurality of hollow tubes 604. Further, when the cartridge 650 is forced against the tissue 601, a portion of tissue 603 is enclosed by the peripheral edge 654. In some cases, the portion of tissue 603 can deflect upwardly (e.g., slightly) into an interior volume defined by the cartridge housing 652, which can also contain the plurality of hollow tubes 604. As shown in FIG. 26B, the cartridge 650 can include a stabilizer 656. The stabilizer 656 can be coupled to the cartridge housing 652. More specifically, the stabilizer 656 can be coupled between opposing ends of the cartridge housing 652 and can extend between opposing ends of the peripheral edge 654. The stabilizer 656 can separate one or more hollow tubes of the plurality of hollow tubes 604 from one or more remaining tubes of the plurality of hollow tubes 604. For example, as shown in FIG. 26B, the stabilizer 656 is positioned between a first segment of the plurality of hollow tubes 604 (e.g., implemented as a single row of hollow tubes) and a second segment of the plurality of hollow tubes 604 (e.g., also implemented as a single row of hollow tubes). In this way, when the cartridge 650 is pressed against the tissue 601 (e.g., with the peripheral edge 654 and the stabilizer 656 also being pressed against the tissue 601), the portion of tissue 603 is separated into two sections 605, 607, each at least partially defined by the stabilizer 656. In this way, by having sections of tissue with smaller surface areas, the skin at each section is more taught and the hollow tubes penetrate the tissue at the section more easily (e.g., as compared to a region having a larger surface area, which can be allowed to deflect). Further, by having the stabilizer and corresponding smaller surface area sections of tissue, the overall force provided to the cartridge 650 to the tissue 601 can be decreased because each section of tissue is better stabilized.

As shown in FIG. 26B, the stabilizer 656 can have a lower surface that is flush with a lower surface of the peripheral edge 654 of the cartridge 650. In this way, when the cartridge 650 is pressed against the tissue 601, the peripheral edge 654 and the cartridge 650 simultaneously contact the tissue 601. Although the peripheral edge 654 is shown as having a square shape, the peripheral edge 654 can have other shapes (e.g., a rectangle, a circle, etc.) For example, the shape of the peripheral edge 654 can be the same as the peripheral shape of the plurality of hollow tubes 604 that form an array. More specifically, when the peripheral shape of the array is a circle, the shape of the peripheral edge 654 can also be a circle. As shown in FIG. 26B, the stabilizer 656 is a bar, having a rectangular shape. However, in other configurations, the stabilizer 656 can have other shapes, particularly when the peripheral edge 654, array, etc., have other shapes. Although only one stabilizer 656 has been shown in FIG. 26B, the cartridge 650 can have a plurality of stabilizers, with each stabilizer being positioned between adjacent segments of the plurality of hollow tubes 604 (e.g., with each segment having a plurality of hollow tubes). For example, the cartridge 650 can include a first stabilizer positioned between a first segment and a second segment and can include a second stabilizer positioned between a second segment and a third segment. This pattern can occur for any number of segments, with the number of stabilizers being X-1 and the number of segments being X.

In some non-limiting examples, the stabilizer 656 can be formed out of various materials. For example, the stabilizer 656 can be rigid and can be formed out of a polymer (e.g., a plastic), a metal, etc. This is advantageous in that when the stabilizer 656 is forced against the tissue 601, the tissue 601 is taught and stabilized. Conversely, if the stabilizer 656 were compliant, the stabilizer 656 would simply curve according to the curvature of the tissue, which would not provide the needed stabilization. Further, then, when the stabilizer 656 is pressed against the tissue 601 (e.g., according to a minimum force, such as described below), the stabilizer 656 can be substantially flat, straight, non-curved, etc.

In some non-limiting examples, the stabilizer 656 (e.g., which can be flat) can be implemented in a different way. For example, the stabilizer 656 can include a plurality of holes that correspond with the plurality of hollow tubes 604. For example, the stabilizer 656 can have a first hole that receives a first hollow tube (e.g., of the plurality of hollow tubes 604), a second hole that receives a second hollow tube (e.g., of the plurality of hollow tubes 604), and so on. More specifically, the stabilizer 656 can have a hole for each hollow tube of the plurality of hollow tubes 604 (e.g., each hole of the stabilizer 656 receives a respective tube of the plurality of hollow tubes 604). In this way, since the holes (e.g., each of which can be circular) are small and thus have a small surface area the section of tissue defined by a hole is further stabilized. In some cases, the stabilizer with the holes can better stabilize the tissue between adjacent tubes; however, the relatively small size of the holes can make interfacing with the respective tubes more difficult. Therefore, the bar configuration can be more desirable. In other configurations, though, each hole of the stabilizer can be substantially larger than the outer width or diameter of a hollow tube. For example, a ratio of the area of the hole of the stabilizer to the area of the outer width (or diameter) of a hollow tube can be greater than substantially 1.15, 1.2, 1.25, etc. In this way, the slightly larger sized hole can avoid the stabilizer inadvertently blocking a given hollow tube, but still providing the maximum possible tissue stabilization (e.g., the lower the ratio the better from a tissue stabilization perspective).

In some non-limiting examples, the position of the stabilizer 656 relative to the peripheral edge 654 can be advantageous. For example, the further a spot of tissue of the portion of tissue 603 is away from the peripheral edge 654, the more difficult the hollow tube is to be inserted into the tissue 601 at the spot (e.g., because the tightness of the tissue decreases away from the peripheral edge 654). Therefore, the stabilizer 656 being positioned at a center of the cartridge housing 652 can provide the best stabilization (e.g., relative to the stabilizer 656). For example, a first group of segments of the plurality of hollow tubes 604 can be positioned on one side of the stabilizer 656 and a second group of segments of the plurality of hollow tubes 604 can be positioned on an opposing side of the stabilizer 656. In some cases, the stabilizer 656 can bisect the cartridge housing 652, and the stabilizer 656 can split or separate the plurality of hollow tubes 604 into two groups having the same number of tubes. In some cases, the stabilizer 656 can be a single stabilizer.

In some non-limiting examples, the stabilizer 656 can be spring loaded, so as to provide a constant force or pressure to the tissue 601 when the cartridge 650 is pressed against the tissue 601. In this case, a spring can be coupled between the stabilizer 656 and the cartridge housing 652. Prior to applying the stabilizer 656 to the tissue 601, the stabilizer 656 can extend past a bottom surface of the peripheral edge 654 a particular distance. In this way, when the cartridge 650 is pressed against the tissue 601, the stabilizer 656 first is forced closer towards the cartridge 650 until the particular distance is reached when the bottom surface of the stabilizer 656 is flush with the bottom surface of the peripheral edge 654. Advantageously, with the spring, further pressing of the cartridge 650 against the tissue 601 does not further load the spring and thus the spring can force the stabilizer 656 against the tissue 601 with a constant pressure or force (e.g., with the distance traveled by the stabilizer 656 and the spring constant dictating the constant pressure or force, at least at the location of the stabilizer 656). Therefore, the stabilizer 656 can provide a pressure or force exerted against the tissue site. In some cases, this pressure is at least substantially 10 lbs., 20 lbs., 30 lbs., 40 lbs., 50 lbs., etc. In some non-limiting examples, including when the system includes multiple stabilizers, each stabilizer can include a respective spring, or one spring can be coupled to multiple stabilizers.

Although the cartridge 650 has been described as stabilizing the tissue, in some cases, including when a system lacks a cartridge, the cartridge housing can be an extension of the housing 602. For example, the cartridge housing can be coupled to or integrally formed with (e.g., creating a single monolithic component) the housing 602. In some cases, the cartridge lacks the hollow tubes 604 and thus the cartridge can simply be a tissue stabilizer (e.g., which can be removably coupled to the housing 602).

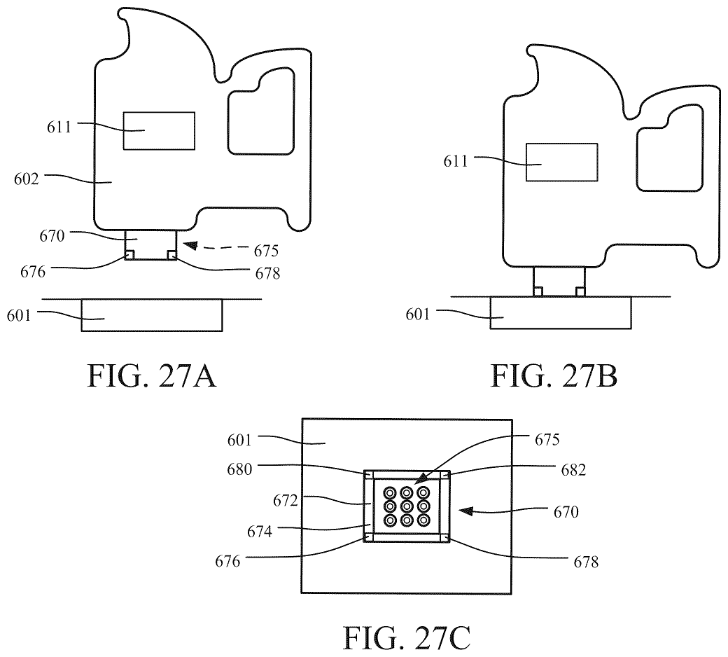

FIG. 27A shows a schematic illustration of the system 600 positioned above the tissue 601 with a different cartridge 670. More specifically, the cartridge 670 is engaged with the housing 602, rather than the cartridge 650. However, it is appreciated that features from the cartridge 650 are interchangeable with the cartridge 650 and vice versa. The cartridge 670 can include a cartridge housing 672 defining a peripheral edge 674, and a plurality of hollow tubes 675. In some cases, the system 600 can include a computing device 611 in communication with all or some of the components of the system 600. The computing device 611 can be coupled to and positioned within the housing 602.

The system 600 or the cartridge 670 can include one or more force sensors (or pressure sensors) coupled to the cartridge housing 672 or otherwise in pressure communication with the cartridge housing 672 or a different component coupled to the cartridge housing 672. In this way, the one or more force sensors can sense the force or pressure that is applied by the cartridge 670 to the tissue 601. For example, as shown in FIG. 27A, the system 600 can include force sensors 676, 678, 680, 682. Each force sensor 676, 678, 680, 682 is coupled to the peripheral edge 674 of the cartridge housing 672 and can have a lower surface that is flush with the lower surface of the peripheral edge 674. Therefore, in some cases, each force sensor 676, 678, 680, 682 can be embedded within the peripheral edge 674 of cartridge housing 672. Although FIG. 27C shows each force sensor 676, 678, 680, 682 being positioned at a respective corner of the peripheral edge 674 (e.g., shapes as a square or a rectangle), each force sensor 676, 678, 680, 682 can be positioned at a respective side of the peripheral edge 674. Further, although the peripheral edge 674 is shown as being a square, when the peripheral edge 674 has other shapes (e.g., a circle), the force sensors 676, 678, 680, 682 can be coupled to the peripheral edge 674 and spaced at intervals around the peripheral edge 674. Although four force sensors has been shown, in other cases, including when the peripheral edge 674 has a different shape than a square or rectangle (e.g., a circle), the one or more force sensors can include other numbers of force sensors (e.g., two, three, five, etc.). Further, although the force sensors 676, 678, 680, 682 are positioned at the bottom of the cartridge 670 (e.g., below the housing 602, below a top of the cartridge 670, etc.) in other configurations, the force sensors 676, 678, 680, 682 can be coupled to the housing 602. In this case, for example, when the cartridge 670 is coupled to the housing 602 (e.g., at the aperture), the cartridge housing 672, and more specifically, a top of the cartridge housing 672 can be forced into contact with the force sensors 676, 678, 680, 682 (or other numbers of force sensors). In this way, when the cartridge 670 is forced against the tissue 601 the force is transferred to these force sensors, without the force sensors being in close or direct contact with the tissue 601. This can provide advantages in that the force sensors can be reused when the cartridge 670 is disposed (e.g., when a different, new cartridge is coupled to the housing 602 for a new procedure), the force sensors are closer to the computing device (e.g., to minimize routing electrical components, to prevent throwing away of the pressure sensors when the cartridge is disposed, etc.), and the force sensors do not have to be spatially integrated or coupled to the cartridge housing 672.

In some non-limiting examples, having at least two force sensors, each positioned on an opposing side of the cartridge can be advantageous. For example, a uniform or constant force applied by the cartridge 670 to the tissue 601 is desirable because, with only one force sensors, for example, the total force can be sufficient for creating taught skin, but that force may not be distributed evenly throughout the cartridge housing 672. Stated another way, there could be locations where inadequate force is applied to the tissue 601 creating a location where the skin is not taught enough. However, when including more than one force sensor, the forces at multiple locations can be compared to ensure that the force is adequate throughout the entire spatial footprint of the cartridge housing 672. Further, with multiple force sensors at opposing ends of the cartridge 670, the computing device 611 can ensure that the cartridge 670 is not angled, which avoids the inclusion of another angle sensor (e.g., the force sensors provide the angle information). For example, if there is a difference in force between the two force sensors, this indicates that the cartridge 670 is angled or is rotated (e.g., the bottom surface of the cartridge is not parallel to the tissue 601), which is not desirable because harvesting can be compromised including all the micrografts not having the same length. Importantly, with another two force sensors at different sides of the cartridge, the system 600 (e.g., via the computing device 611) can ensure that the cartridge is not angled or rotated about a different degree of freedom. In other words, having four force sensors, with pairs being positioned at opposing sides, can provide information about whether the cartridge is angled relative to the tissue 601 about multiple different degrees of freedom (e.g., axes, each defined by an intersection with a pair of force sensors).

In some non-limiting examples, the system 600 can ensure that adequate force is applied throughout the spatial footprint of the cartridge and can ensure that the cartridge is not angled or rotated during harvesting of portions of tissue from the tissue 601 (e.g., when the cartridge 670 is pressed against the tissue 601). For example, the computing device 611 can receive a force value (or force information) from each force sensor 676, 678, 680, 682. The computing device 611 can then compare each force value to a threshold force (e.g., 10 lbs., 20 lbs., 30 lbs., 40 lbs., 50 lbs., etc.). If the computing device 611 determines that any (or all) of the force values are below the threshold force, the computing device 611 can transmit an alert (e.g., to another computing device), notification, etc., to a user and can prevent or lock the plurality of hollow tubes 675 from being inserted into the tissue 601. If, however, the computing device 611 determines that any (or all) of the force values are above the threshold force, the computing device 611 can allow or permit the insertion of the hollow tubes 675 into the tissue 601 (e.g., the computing device 611 can cause the actuation system to insert the plurality of hollow tubes 675 into the tissue 601, such as, segment by segment). Similarly, the computing device 611 can compare a force value from one force sensor (e.g., the force sensor 676) to the force value from a different force sensor (e.g., the force sensor 680), and can complete this for each pair of force sensors. If the computing device 611 determines that any (or all) of the pairs of force values are greater than a difference threshold (e.g., ±3 Newtons), by, for example, subtracting a pair of force values and comparing this resulting value to the difference threshold, the computing device 611 can transmit an alert, notification, etc., to a user, and can prevent or lock the plurality of hollow tubes 675 from being inserted into the tissue. If, however, the computing device 611 determines that any (or all) of the pairs of force values are less than a difference threshold (e.g., ±3 Newtons), by, for example, subtracting a pair of force values and comparing this resulting value to the difference threshold, the computing device 611 can transmit an alert, notification, etc., to a user, and can prevent or lock the plurality of hollow tubes 675 from being inserted into the tissue 601. In some cases, the computing device 611 can allow the plurality of hollow tubes 675 to be inserted into the tissue 601 if both one or more force values are less than a threshold force (e.g., indicating adequate force applied) and one or more pairs of force values are less than a difference threshold (e.g., indicating adequate angling, such as no angle present).

Although the cartridge has been described as stabilizing the tissue, in some cases, including when a system lacks a cartridge, the cartridge housing can be an extension of the housing 602. For example, the cartridge housing can be coupled to or integrally formed with (e.g., creating a single monolithic component) the housing 602. In some cases, the cartridge lacks the hollow tubes 604 and thus the cartridge can simply be a tissue stabilizer (e.g., which can be removably coupled to the housing 602). In some non-limiting examples, therefore, the one or more force sensors can be positioned between such a “cartridge” and the housing 602 so as to measure the force applied by the “cartridge” to the tissue 601.

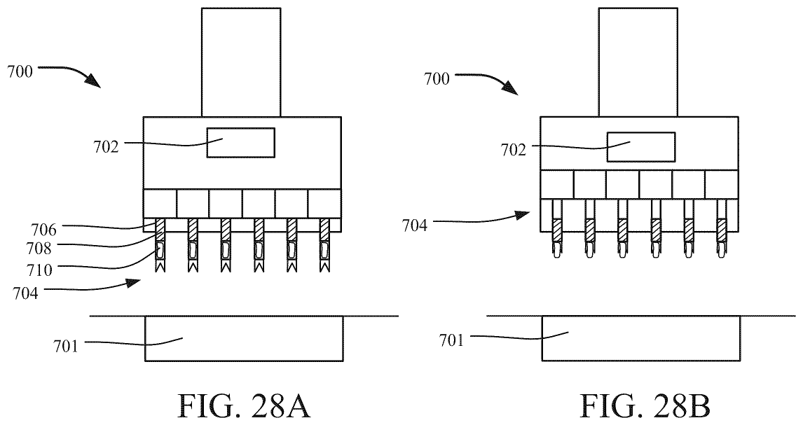

FIG. 28A shows a schematic illustration of an example of a system 700 (e.g., a skin grafting system), which can be a specific implementation of other system described herein. The system 700 can include other components from other systems, but is shown as being simplified, demonstrating scattering of portions of tissue. For example, the system 700 includes an actuation system 702 and a plurality of hollow tubes 704. Each hollow tube of the plurality of hollow tubes 704 can include a respective pin positioned therein, such as described above. Each of these pins can be coupled to an actuator (e.g., a single actuator) or spring to drive translational movement of each pin within the respective hollow tube (e.g., to adjust the axial length of tissue taken during harvesting). As shown in FIG. 28A, the system 700 has previously harvested tissue and therefore each hollow tube of the plurality of tubes includes a respective portion of tissue trapped therein (e.g., a micrograft). For example, for demonstration purposes, the plurality of hollow tubes 704 can include a hollow tube 706 including a pin 708 positioned therein (e.g., within a lumen of the hollow tube 706), and a portion of tissue 710 (e.g., skin tissue, a micrograft, etc.) also positioned within the hollow tube 706 (e.g., also within the lumen of the hollow tube 706, below the pin 708). Each hollow tube of the plurality of hollow tubes 704 can be similar to the hollow tube 706 (e.g., including the pin 708 and the portion of tissue 710).

In some non-limiting examples, and as shown in FIG. 28A, the system 700 (e.g., the housing thereof, such as the handheld device, the cartridge, etc.) can be positioned above a recipient site 701 (e.g., skin tissue, a wound, a prepared site for transfer, etc.). In this way, none of the plurality of hollow tubes 704 contact the recipient site 701, which is advantageous for a number of reasons. First, the sharp ends of the hollow tubes 704 do not further aggravate the recipient site 701 (e.g., causing blood, which can damage the site and can obscure the view). Second, depositing of the portions of tissue out of each tube can be easier if elevated above the recipient site 701 (e.g., because the hollow tubes can be continuously extended and retracted without damaging the recipient site 701 to expel the tissue portions which can be sticky and can temporarily adhere to the walls of a given hollow tube). Further, this scattering process in which the system 700 hovers above the recipient site 701 (e.g., without the hollow tubes 704 contacting the tissue) can easily deposit the portion of the tissue (e.g., tissue portions) onto the recipient site 701 without the orientation of the tissue portions being important. In other words, after the tissue portions are deposited on the tissue at various orientations (e.g., flat, angled, etc.) the tissue portions automatically “seed” the creations of new tissue columns at the recipient site, such that when healed, the tissue columns are at the correct orientation when incorporated into the recipient site 701. Stated another way, the deposited tissue portions provide the seeds necessary for the migration of different cell types that migrate to the correct layer/location within the recipient site. As shown in FIG. 28B, the system 700, when raised above the recipient site 701, can translate the hollow tubes 704 upwardly (e.g., retracting the hollow tubes) and back downwardly (e.g., to the position shown in FIG. 28A), repeatedly, until the tissue portions are deposited onto the recipient site 701. In some cases, the actuation system 702 can advance and retract all of the hollow tubes 704 at the same time. For example, an actuator (e.g., a vertical actuator) can retract and advance all of the hollow tubes 704 at once. As another example, including when there are multiple actuators (e.g., multiple vertical actuators) all the actuators can advance and retract the segments at the same time thereby retracting and advancing all the hollow tubes 704 at the same time. When retracting and advancing the hollow tubes 704, each pin can be in a fixed position, such that each pin helps push the tissue portion out of the respective hollow tube when the hollow tube is retracted and advanced.

In other cases, the hollow tubes 704 can be stationary and the pins can be advanced to extrude the tissue portions out of the respective hollow tube. For example, with the hollow tubes 704 stationary the computing device (not shown) can cause all the pins to advance and retract quickly, in a similar way as the hollow tubes being advanced and retracted to remove each tissue portion from each hollow tube. In other cases, the pins can simply be advanced until a distal end of each pin is positioned outside of a respective hollow tube to remove the tissue portions from the hollow tubes.

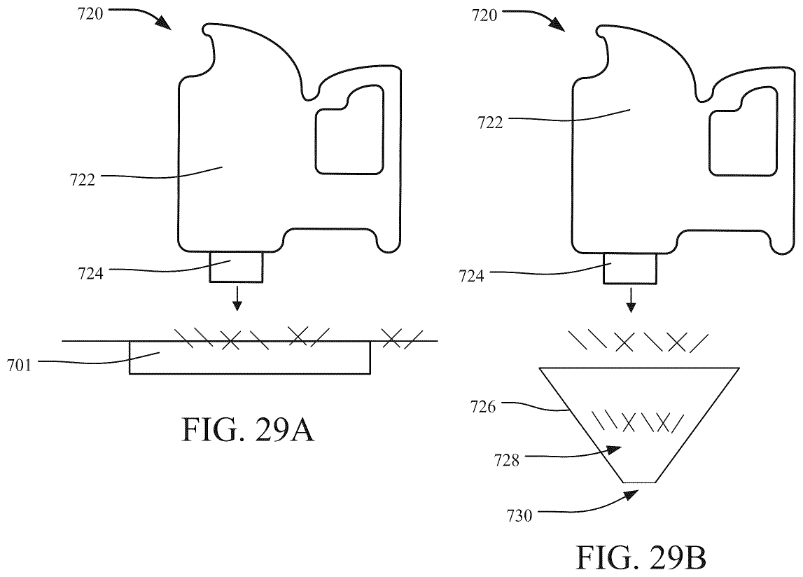

FIG. 29A shows a schematic illustration of an example of a system 720 (e.g., a skin grafting system), which can be a specific implementation of other system described herein. The system 720 can include other components from other systems, but is shown as demonstrating scattering of portions of tissue. The system 720 can include a housing 722 and a plurality of hollow tubes included in a cartridge 724. FIG. 29A shows a similar configuration in which the system 720 is raised above the recipient site 701 and the tissue portions (e.g., micrografts) are deposited directly onto the recipient site 701. In some cases, when the hollow tubes are retracted and extended to remove the tissue portions trapped within the hollow tubes, this action can cause the tissue portions to disperse further than desired. For example, tissue portions can be deposited in locations outside the recipient site 701. Therefore, in some cases, it can be desired to capture the tissue portions before depositing them onto the recipient site 701.

FIG. 29B shows a schematic illustration of the system 720 with a scatter aid to capture tissue portions before applying them onto a recipient site. For example, the system 720 can include a collector 726. As shown in FIG. 29B, the collector 726 can include a interior volume 728 to receive and retain the tissue portions. Further, the collector 726 can have a cross-sectional area that decreases from a first end of the collector 726 to an opposing second end of the collector 726. Therefore, the first end of the collector 726 can be wider (or can have a greater dimension, such as diameter) than the second end of the collector 726. In some cases, this first end of the collector 726 can be positioned closer to the hollow tubes than the second end of the collector 726. In some cases, and as shown in FIG. 29B, the first end of the collector 726 is wider than the cartridge 724 or wider than the plurality of hollow tubes (e.g., the width of the array that defines the hollow tubes). In this way, the hollow tubes (and the cartridge) can be inserted into the interior volume 728 of the collector 726, such as during a scattering process to capture the tissue portions within the collector 726. In some cases, the second end of the collector 726 can be enclosed (e.g., to capture and retain the tissue portions). In other configurations, the collector 726 can include a hole 730 positioned at the second end. In some cases, this hole 730 can have a dimension that is the same (or substantially the same) as a corresponding dimension of the tissue site. For example, the hole 730 can have a diameter that is the same as a diameter of the recipient site 701. As another example, the hole 730 can have a length that is the same as the length of the recipient site 701. In this way, the hole 730 can be positioned above the recipient site 701 during a scattering process to help guide the tissue portions onto the recipient site 701. In some cases, therefore, the collector 726 can be removably coupled to the housing 722 of the system 720. In other cases, the collector 726 can be removably coupled to the cartridge 724 (e.g., the cartridge housing thereof) In this way, after a harvesting is complete, the collector 726 can be coupled to the housing 722 (or cartridge 724) such that the plurality of needles (and the cartridge 724) are positioned within the interior volume 728 of the collector 726 during scattering. At this point, the system 720 with the collector 726 coupled thereto can be positioned over the recipient site 701, and the hole 730 of the collector 726 can be aligned with the recipient site 701. Then, the scattering process can proceed with the tissue portions being deposited into the collector 726, being guided by the collector 726, and deposited out of the hole 730 onto the recipient site 701. Once the scatter process is completed, the collector 726 can be decoupled from the housing 722 (e.g., to be disposed of).

In some non-limiting examples, the interior surface of the collector 726 can include an antifriction layer, coating, etc., such as, for example, Teflon® (e.g., polytetrafluoroethylene), a nonstick coating (e.g., BAM). In this way, the tissue portions do not stick to the surface of the collector 726, but instead glide off until the tissue portions reach the second end that is enclosed or are emitted out of the hole 730 and onto the recipient site 701. In some non-limiting examples, the collector 726 can be a funnel, or have a cone shape (e.g., a frustoconical shape). However, in other configurations, the collector 726 can have other shapes, such as, for example, a bowl, a plate, etc. Although the system 720 has been described as being used with a cartridge, in other configurations, as described above, the system 720 need not include a cartridge.

In some configurations, the system 720 can include a force sensor, a pressure sensor, a scale, etc., which can be in communication with a computing device of the system 720 (or a different computing device). The scale (or other sensor), which can be positioned under the collector 726 (e.g., at the position of the hole 730 if the hole 730 is absent or plugged by a cover) can measure the mass or the weight of all the tissue portions deposited within the collector 726. In this way, the computing device, can determine the number of tissue portions deposited from the cartridge 724 by using a standard reference of the mass or weight of a single tissue portion. Correspondingly, then, the computing device can determine whether all (or a desired number) of tissue portions have been deposited into the collector 726. For example, the computing device can receive the number of hollow tubes (e.g., from a user input, a cartridge identifier associated with the number of hollow tubes, etc.) and can determine the ideal mass or weight of the total number of harvest tissue portions (e.g., by multiplying the reference value weight or mass per tissue portion by the number of hollow tubes). Then, the computing device can determine whether the current weight or mass (e.g., from the scale) deviates from the ideal weight or mass by a threshold percentage or amount (e.g., 20 percent). If the computing device determines that the current mass or weight is less than the threshold percentage (e.g., 80%) of the ideal weight or mass, the computing device can cause the scattering system of the system 720 to implement an additional scattering process (e.g., because the lower than ideal mass indicates that the tissue portions are still trapped in the hollow tubes). If however, the computing device determines that the current mass or weight is greater than or equal to the threshold percentage of the ideal weight or mass, the computing device can notify a user indicating that the scattering process is completed. In some cases, after the computing device determines that the weight or mass is below a threshold and implements a further scattering process, the computing device can subsequently determine whether the weight or mass is still below the threshold. This process can proceed a number of times (e.g., three) at which point the computing device can end the loop (e.g., greater than the number of times) to avoid being stuck in the loop (e.g., because the further scattering processes are not depositing additional tissue portions, which means an error has occurred including an inadequate harvesting sequence).

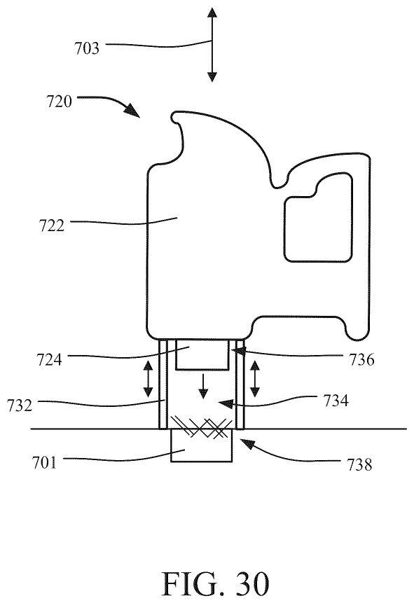

FIG. 30 shows the system 720 with a different scatter aid, to help direct tissue portions during a scattering process. The system 720 can include a scatter aid 732 that can be removably coupled to the housing 722. Similarly to the collector 726, the scatter aid 732 can include an interior volume 734 that can receive and enclose the hollow tubes and the cartridge when the scatter aid 732 is coupled to the housing 722. As shown in FIG. 30, the scatter aid 732 can include holes 736, 738 that can be positioned at opposing ends of the scatter aid 732. For example, the hole 736 can be positioned near the housing 722 when the scatter aid 732 is coupled to the housing 722, while the hole 738 can be positioned at the recipient site 701. In some cases, a dimension of the holes 736, 738 (e.g., a diameter, a width, etc.) can be same or substantially the same, while in other cases, such as the like the collector 726, the holes 736, 738 can have different sizes. As shown in FIG. 30, a height of the scatter aid 732 can be adjustable (e.g., along an axial axis 703 shown in FIG. 30), so as to position the hollow tubes and the cartridge at different heights away from the recipient site 701. For example, the scatter aid 732 can have legs that are adjustable, can have telescopic segments to adjust the height, etc.

In some non-limiting examples, the scatter aid 732 can surround the recipient site 701 (e.g., entirely surround the recipient site) and the scatter aid 732 can be fully enclosed so as to prevent any tissue portions from being directed away from the recipient site 701. In some cases, along with the scatter aid 732 better directing the tissue portions at the recipient site 701, the scatter aid 732 also can provide support for the system during the scattering process. For example, the scatter aid 732 can stabilize the system 720 during the scattering process, which can prevent a user from having to expend energy to hover the system 720 above the recipient site 701. Similarly to the collector 726, an inner surface of the scatter aid 732 can also include an antifriction layer or coating to help prevent the tissue portions from sticking to the walls of the scatter aid 732. In some configurations, before a scattering process but after the hollow tubes have harvested and retain tissue portions, the scatter aid 732 can be coupled to the housing 722 with the scatter aid 732 surrounding the hollow tubes (and the cartridge 724). Then, the scatter aid 732 can be positioned over the recipient site 701, with the hole 738 aligned with recipient site 701, and the hollow tubes can be retracted and advanced until the tissue portions are emitted from the hollow tubes and are directed by the scatter aid 732 to the recipient site 701.

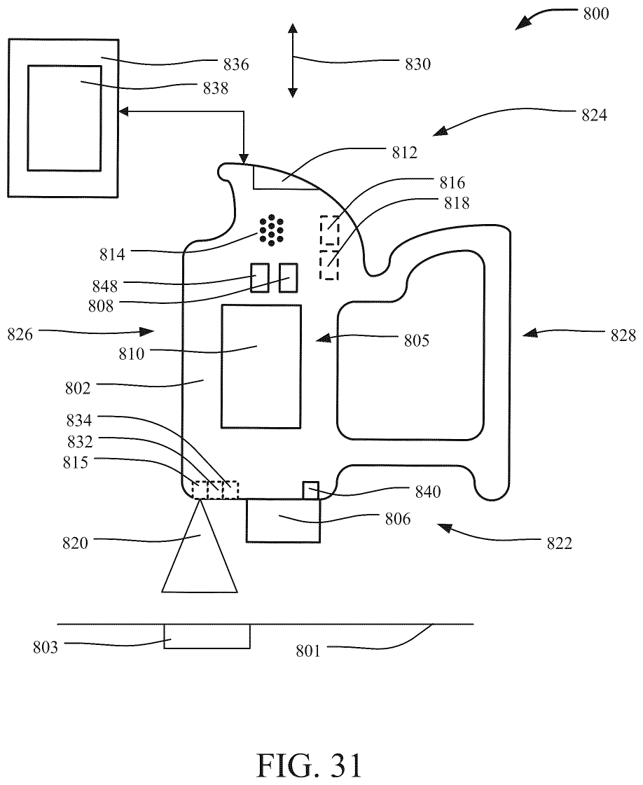

FIG. 31 shows a schematic illustration of an example of a system 800 (e.g., a skin grafting system), which can be a specific implementation of other system described herein. For example, the system 800 can include a housing 802, a plurality of hollow tubes implemented as a cartridge 806, an actuation system 805, a computing device 808, etc. The system 800 can include display devices 810, 812, a speaker 814, and an imaging device 815. Each display device 810, 812 can be an interactive display, such as a touch screen. Thus, each display device 810, 812 can be configured to present information to the user and receive commands from the user (e.g., as a user input). The speaker 814 can be configured to provide audio output (e.g., beeps, speech output, etc.) to the user. The audio output can include feedback or non-feedback sounds (e.g., active noise cancellation to reduce the sound of the solenoid or other actuator firing). In this regard, system 800 (e.g., the handheld device thereof) can include a microphone 816 that can be positioned within the housing 802 of the system 800 and can be in close proximity to the solenoid. In this way, the computing device 808 (e.g., within the housing 802) can receive an audio signal (e.g., a sound recording, audio data, etc.) from the microphone 816 indicative of the solenoid firing. The computing device 808 can then modify the audio signal (e.g., filtering, amplifying, etc.) including inverting the audio signal, phase shifting the audio signal (e.g., to compensate for a delay in receiving the audio signal from the solenoid firing) to then be provided to a speaker 818 positioned within the housing 802 (e.g., in the same chamber as the solenoid, microphone 816, etc.). In this way, the sound emitted from the speaker 818 can provide noise canceling during actuation of the solenoid. Although noise-canceling can be implemented to mitigate noises provided by a solenoid, these components and respective processes can be implemented using on any number or types of actuators used. In some configurations, the speaker 818 can face the solenoid (or other actuator, such as a different type of actuator, such as a vertical actuator). In this way, sound emitted by the speaker 818 can be pointed at the solenoid (or other actuator as applicable) to better target the source of sound desired to be canceled.

In some configurations, the system 800 (and more specifically the computing device 808 and the speaker 818) can perform noise cancelation without requiring use or continuous use of the microphone 816. For example, if firing of the solenoid (or other actuator) has a unique, consistent, sound signature (which is likely in the case of a solenoid or other actuator that repeats the same noise after each firing), this signature can be used to perform noise canceling when the actuator is firing (e.g., by the computing device 808 applying an inverted noise signature to the speaker 818). In some cases, although the system 800 has been described has having a speaker 818 positioned within the housing 802 and facing the solenoid, in other configurations, the system 800 can include multiple speakers (e.g., each implemented in a similar way to the speaker 818). For example, a first speaker can be positioned to one side of the solenoid (or other actuator that drives hollow tubes into tissue) facing the solenoid, while the second speaker can be positioned to a second side of the solenoid also facing the solenoid. The computing device 808 then can provide an audio signal indicative of the solenoid firing (e.g., an inverted audio signal) to the first speaker and the second speaker to cancel the sound emitted by the solenoid. In this way, since the solenoid can be idealized as a point source for audio signals, in some cases, having multiple speakers can further minimize the noise produced by the solenoid by further canceling the noise. In some cases, therefore, the system 800 can include three, four, five, etc., numbers of speakers to cancel the noise from the solenoid. In some non-limiting examples, mitigation of noise by the solenoid or other actuator can be implemented passively. For example, a material can be provided within the housing 802 to decrease sound propagation therethrough. Such a material can be insulation, other fibers, a sound dampener, etc.

In some non-limiting examples, the bulk of the housing 802 can make visibility of the cartridge 806 difficult due to the orientation of the housing 802 and the cartridge 806 (and thus hollow tubes therein). For example, a user typically views the system 800 from a top view. In some cases, such as during harvesting, the blockage in visibility of the cartridge 806 and hollow tubes is less problematic because typically there are large swaths of donor tissue (e.g., skin tissue) to harvest from (and the cartridge or other housing can be placed directly at the harvesting site). In other words, the span of the donor tissue is far greater than the size of the cartridge 806. However, during a scattering process, since the recipient site 803 can be quite small, it is important that the hollow tubes (e.g., the cartridge 806) are aligned with the recipient site 803 so as to make sure the tissue portions are deposited on the recipient site 803 (and not at other portions of the tissue that are not intended to receive tissue portions, such as healthy regions of tissue). Therefore, including an imaging device 815 to provide one or more images of the tissue 801 including the recipient site 803 can be desirable, such as to properly align the hollow tubes (and thus the tissue portions retained by the hollow tubes) with the recipient site 803 during scattering. Therefore, the imaging device 815 can be configured to capture one or more images (including a video) of an area below the housing 802, below the hollow tubes, below the cartridge 806, etc. For example, as shown in FIG. 31, a field of view (“FOV”) 820 of the imaging device 815 can extend away from a bottom side 822 of the housing 802 (e.g., which interfaces with the cartridge 806) and can extend away from the top side 824 of the housing 802. Further, as shown in FIG. 31, the imaging device 815 can be positioned in front of the plurality of hollow tubes and the cartridge 806. For example, the imaging device 815 can be positioned closer to the front side 826 of the housing 802 than a rear side 828 of the housing 802 (e.g., that includes a handle and a slot). Correspondingly, the imaging device 815 can face downwardly away from the sides 822, 824, such that an optical axis of the FOV 820 is substantially parallel with an axis 830 (e.g., a longitudinal axis) of the housing 802 (e.g., where the axis 830 intersects the side 822, 824). Although the imaging device 815 is shown as being coupled to the bottom side 822 of the housing 802, the imaging device 815 can be coupled to the front side 826 of the housing 802 (e.g., while also facing downwardly). Further, while there are advantages to the location of the imaging device 815 in front of the hollow tubes (e.g., for spatial reasons and with the image leading the actual position of the hollow tubes because the imaging device 815 is in front of the hollow tubes and it is more natural to move the system 800 forward as opposed to other directions), the imaging device 815 can be coupled to the housing 802 at different locations (e.g., positioned behind the hollow tubes). In some cases, the imaging device 815 can be positioned centrally relative to the hollow tubes and cartridge 806. For example, the imaging device 815 can bisect the hollow tubes into two substantially equal groups and similarly can bisect the cartridge into two even halves. In this way, the computational load on the computing device 808 can be lowered because the position of the imaging device 815 is better tied to the position of the hollow tubes and cartridge 806. In some non-limiting examples, the imaging device 815 can be positioned to acquire the one or more images of a target area where a harvesting, scattering, etc., process is being performed. In some non-limiting examples, the FOV 820 does not intersect with the cartridge 806.

Each display device 810, 812 can be implemented in a similar manner. For example, each display device 810, 812 can be or include any display panel configured to display images, text, etc., to the user. In some examples, each display device 810, 812 can be a flat screen display, such as a liquid crystal display (LCD), a light emitting diode (LED) display, an organic light emitting display (OLED), a quantum dot display, and the like. Each display device 810, 812 can be configured to provide one or more images to the user (e.g., during a scattering process). For example, each display device 810, 812 can show a live view (or a real-time view, such as acquired from receiving and sensing light from the FOV 820) of the procedure target area, which can include a graphical user interface (“GUI”) overlay. The GUI overlay can provide information to the user. In one example, the GUI overlay can provide a level display to assist the user in ensuring that the system 800 (e.g., the housing 802, which can be defined as the handheld device including the housing 802) is held at a proper angle to the harvest site (e.g., using the force sensors as described above).

The imaging device 815 can be or include any image sensor configured to receive incident electromagnetic radiation (e.g., visible light, infrared radiation, etc.) and generate electronic signals in response thereto. The imaging device 8116 can, in implementations, include a plurality of individual sensors to provide stereoscopic imaging (e.g., to acquire 3D imaging data). In some examples, the imaging device 815 can include at least one complementary metal-oxide-semiconductor (“CMOS”) sensor, a charge couple device (“CCD”), and the like. The imaging device 815 can include or be associated with a light emitting element, which can be used to project structured light that is detected by the imaging device 815. For example, the system 800 can include light sources 832, 834, each of which can be coupled to the housing 802 and can be positioned at the bottom side 822 of the housing 802. Although the imaging device 815 is shown being positioned in front of the light sources 832, 834, each light source 832, 834 can be positioned in front of the imaging device 815. Further, in some cases, the imaging device 815 can be positioned between the light sources 832, 834 (e.g., which can better illuminate the FOV). Regardless of the configuration, the light sources 832, 834 (or one light sources, or more than two light sources) each can emit light to illuminate at least a portion of or the entire FOV 820. This can facilitate easier image acquisition when the ambient light levels low. In some non-limiting examples, while FIG. 31 shows the imaging device 815 being coupled to a bottom side 822 of the housing 802, in some implementations the imaging device 815 can be coupled to the cartridge 806 (e.g., to be disposed of after use along with the cartridge 806)

Each display device 810, 812 (e.g., in combination with the imaging device 815) can be used as a scattering aid to alleviate issues that may occur if the spatial bulk of the system 800 (and more specifically the housing 802) covers up visibility of the cartridge 806, such that it becomes difficult for the user to determine a good coverage. Thus, each display device 810, 812 can eliminate the need for an additional assistant to help guide and ensure that the cartridge 806 is positioned correctly during a scattering procedure. In some cases, the light source 832 can emit a light pattern towards and on the tissue 801 to visually show the actual location of the cartridge and scatter. In some cases, this light pattern can be a laser pattern and can have different shapes, such as an outer peripheral shape that corresponds to the outer peripheral shape of the cartridge 806 (e.g., a square). In other configurations, the light pattern can be an “X”, a cross, etc.

As shown in FIG. 31, the display device 810 can be positioned on a lateral side of the housing 802, while the display device 812 can be positioned on the top side 824 of the housing 802. In other cases, a display device (e.g., the display device 810) can be positioned on the rear side 828 of the housing 802. In some cases, the top side positioning of the display device 812 can be advantageous in that during a scattering process, a user is orienting the system 800 as is shown in FIG. 31, with the user's head positioned above the system 800. In this way, with the display device 812 on the top side, the display device 812, showing a real-time image of the tissue 801 can be viewed by a user at the same time as the actual top down view of the system 800. In other words, the coordinate system of the display device 812 and the coordinate system of the imaging device 815 are close together (e.g., with the position of the imaging device 815 being translated downwardly and to the side of the display). In this way, with the system 800 in the orientation shown in FIG. 31, side to side, rear to back, back to front, etc., motion of the system 800 is shown on the display device 812 as images translating the current FOV in the same direction of movement of the system 800.

In some non-limiting examples, the system 800 can include a computing device 836 that is separate from the housing 802. The computing device 836 can include a display device 838 and the computing device 836 can be in communication (e.g., bidirectional communication) with the computing device 808. In some cases, the computing device 836 can be a tablet, a monitor, a smartphone, etc. The computing device 836 can receive the one or more images acquired by the imaging device 815 and can present them on the display device 838. In this way, a practitioner can view the real time FOV of the tissue 801 in a similar way as the other display devices 810, 812. In some cases, however, the housing 802 lacks any display devices coupled thereto, and thus the display device 838 can function as one (or the only) display devices of the system 800 (e.g., the system 800 only includes the display device 838).

In some non-limiting examples, and as described above, the computing device 808 of the system 800 can be in communication (e.g., bidirectional communication) with some or all of the components of the system 800. Therefore, the system 800 can receive information from each of these components and can control each of these components (e.g., cause these components to implement a particular task). In some cases, the computing device 808 can adjust or otherwise shift the coordinate system of the imaging device 815, such that the FOV 820 is virtually shifted to align with a center of the plurality of hollow tubes (e.g., the center of the array of hollow tubes) or a center of the cartridge. For example, the computing device 808 can shift the coordinate system of the imaging device 815 to correspond to the coordinate system of the hollow tubes (e.g., the array) or the coordinate system of the cartridge. As a more specific example, the computing device can shift the coordinate system of the imaging device 815, based on the known position between the imaging device and the hollow tubes (or cartridge). For example, the coordinate system of the imaging device 815 can be adjusted rearwardly (e.g., only translated rearwardly), such as when the imaging device 815 is centered with the hollow tubes (and cartridge). In some configurations, the computing device 808 can acquire an image of the FOV 820, can shift the image according to the adjusted coordinate system of the imaging device 815, and can then present the shifted image on any of the displays. In some cases, the FOV 820 at the intersection between the FOV 820 and the tissue surface 801 can be larger than the recipient site 803 (e.g., to accommodate for the coordinate system shift of the imaging device 815).

In some non-limiting examples, the computing device 808 can receive one or more parameters (e.g., each in the form of a user input from the user interacting with a display device, such as the display device 810) and can determine a number of harvesting processes and scattering processes to be implemented based on the one or more parameters (e.g., with the number of harvesting and scattering processes being the same). For example, the computing device 808 can receive a size of the recipient site such as a wound (e.g., previously measured by a practitioner, using for example, a tape measure, ruler, etc.) from a user input (e.g., by the user interacting with a display device 810). Then, the computing device 808 can determine the number of harvesting and scattering processes based on the size of the wound (e.g., one of the one or more parameters). For example, a standard size or area can be used (e.g., the area of the cartridge) to be used to determine the number of harvesting and scattering processes. In this case, the computing device 808 can determine the number of harvesting and scattering processes from dividing the area of the recipient site by the standard size. In some cases, this dividing results in a decimal number, in which case the computing device 808 can round the decimal number to the nearest whole number, which is the number of harvesting and scattering processes to be completed (e.g., because it is better to deposit more tissue portions than less onto a recipient site). In some cases, the one or more parameters can be the number of hollow tubes per the standard size, which as described below, can be received by the computing device 808 via a user input or by data associated with the cartridge. In this way, the number of harvesting and scattering processes can be based on the number of hollow tubes (e.g., the density of hollow tubes), in which a higher density of hollow tubes corresponds to lower numbers of harvesting and scattering processes and vice versa.

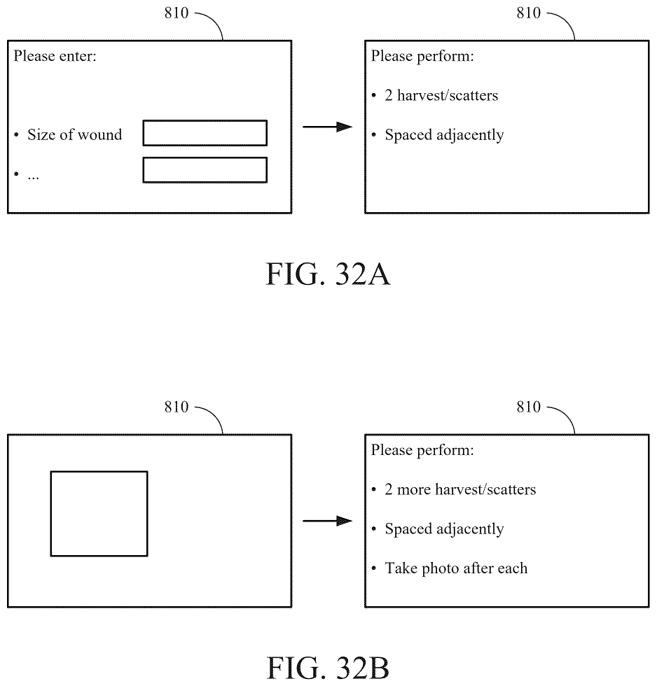

In some non-limiting examples, once the computing device 808 determines the number of harvesting and scattering sequences (e.g., one, two, three, etc.). This information can be displayed on at least one display (e.g., the display device 810) to guide the practitioner. Further, the number harvesting sequences and the number scattering sequences to be completed can be displayed on the display. For example, after each harvesting sequence has been completed (e.g., by the hollow tubes being retracted to a home position), the number of harvesting sequences can be decreased by one and subsequently displayed. Similarly, after each scattering sequence has been completed (e.g., by the hollow tubes being advanced and retracted quickly a number of times), the number of scattering sequences can be decreased by one and subsequently displayed. These processes can be completed until both numbers reach zero. At this point, the computing device 808 can lock the system 800 to prevent further harvesting or scattering. FIG. 32A shows an example of an interaction between the display device 810 and the user. The left screen shows an example input request presented on the display device 810, particularly for the size of the wound. The right screen shows an example output presented on the display device 810. The output response advises the user as to the types of operations to be performed, parameters of the operations, etc.

FIG. 32B shows another example of an interaction between the display device 810 and the user. The left screen shows an example procedure visualization presented on the display device 810. In the illustrated example, the procedure visualization is a view of the procedure target area as captured by the imaging device 815 (either based on a previous image capture or a real-time view). The right screen shows an example output presented on the display device 810. The output advises the user as to the types of operations that remain to be performed or parameters of the operations.

In some non-limiting examples, the system 800, and more specifically, the computing device 808 can determine that the cartridge 806 is coupled to the housing 802 and can correspondingly determine that the cartridge 806 is decoupled from the housing 802. For example, the system 800 can include a sensor 840 coupled to the housing 802 and configured to sense when the cartridge 806 is coupled to (or decoupled from) the housing 802. In some cases, the sensor 840 can be an optical sensor (e.g., a photoresistor, a phototransistor, etc.) and can sense coupling of the sensor by blocking of the optical sensor (e.g., less ambient light is received by the optical sensor when the cartridge is blocking ambient light from reaching the optical sensor) and can sense decoupling of the sensor by unblocking of the optical sensor (e.g., more ambient light is received by the optical sensor when the cartridge is not blocking ambient light from reaching the optical sensor). In some cases, the sensor 840, including when implemented as an optical sensor, can be positioned to face the aperture (e.g., the loading aperture) of the housing 802. The sensor 840 can be implemented in other ways, such as being a switch, a Hall-Effect sensor, etc.

In some non-limiting examples, variations in the patient's characteristics (e.g., gender, skin tightness, age, race, ethnicity, etc.), variations in the properties of the harvest site, etc., can make particular hollow tubes better suited than others during harvesting. Therefore, a system (e.g., a skin grafting system) can include different cartridges (e.g., two, three, four, etc.), each having at least one different characteristic. For example, the characteristics can include the number of hollow tubes, the number of segments of the hollow tubes, the density of the hollow tubes (e.g., the distribution of the hollow tubes within the cartridge housing), the perimeter defined by the number of hollow tubes (e.g., that are actuatable), the length of each extension of each hollow tube of the plurality of hollow tubes (e.g., the lengths of the two points at the distal end of the tube), etc. In some cases, to distinguish different cartridges from each other, each cartridge can include a cartridge identifier (e.g., an alphanumeric code, a numerical code, etc.). For example, the cartridge identifier can be associated with a list of characteristics for that specific cartridge (e.g., with a database, spread sheet, etc., that associates a cartridge identifier with the list of characteristics or parameters for that cartridge identifier), and the computing device can, after receiving the cartridge identifier, extract the characteristics from the cartridge identifier or otherwise ensure that the cartridge identifier matches with a previously determined cartridge identifier. In some cases, the cartridge identifier can be extracted in different ways. For example, each cartridge can include a machine readable code (e.g., a barcode, a QR code, etc.) positioned on a surface of the cartridge that encodes the cartridge identifier, each cartridge can include an electronic component coupled thereto that stores or otherwise encodes the cartridge identifier (e.g., an RFID tag, a Near Field Communication chip, etc.), etc.

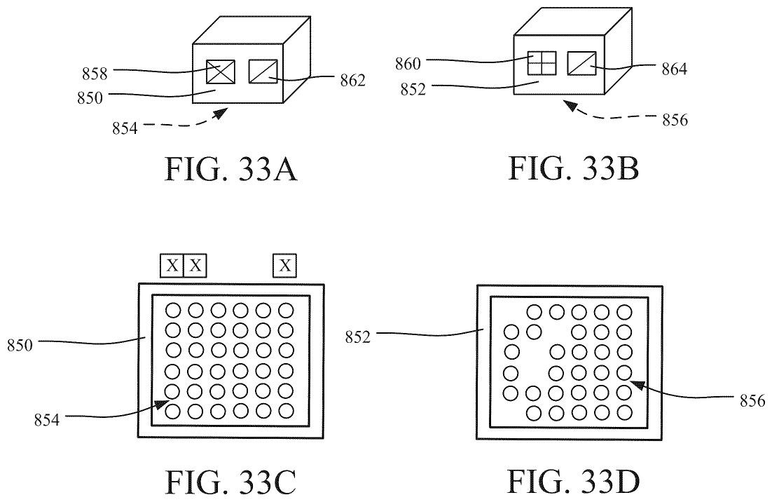

FIGS. 33A-33D show schematic illustrations of different cartridges that illustrate each cartridge having one or more different characteristics or parameters. These cartridges will be described with reference to the system 800, however, these cartridge can be applicable to other systems described herein (and vice versa). FIG. 33A shows a cartridge 850, while FIG. 33B shows a cartridge 852. Each cartridge 850, 852 is different from each other, but each cartridge 850, 852 can be removably coupled to the housing 802 (e.g., at the aperture, a loading aperture, etc.), and thus can be selected to be used with the system 800 during a particular procedure. Therefore, in some cases, and as shown in FIGS. 33A and 33B, each cartridge 850, 852 can have substantially the same spatial footprint. In particular, the cartridge housing of each cartridge 850, 852 can be substantially the same, including particular components thereof, such as the flange that extends around each cartridge housing.

Each cartridge 850, 852 can have a plurality of hollow tubes. For example, the cartridge 850 includes a plurality of hollow tubes 854, while the cartridge 852 includes a plurality of hollow tubes 856. Each cartridge 850, 852 can have a plurality of characteristics (e.g., the number of hollow tubes, the number of segments of the hollow tubes, the density of the hollow tubes, the perimeter defined by the number of hollow tubes (e.g., that are actuatable), the length of each extension of each hollow tube of the plurality of hollow tubes (e.g., the lengths of the two points at the distal end of the tube), etc. Further, as described above, each cartridge 850, 852 can have at least one common or corresponding characteristic that is different. For example, the cartridge 850 can have a first characteristic (e.g., the number of hollow tubes) and a first parameter for the first characteristic (e.g., the specific number of hollow tubes). Correspondingly, the cartridge 850 can have a second characteristic (e.g., the number of hollow tubes) and a second parameter for the second characteristic (e.g., the specific number of hollow tubes). In this case, the first characteristic can be the same as the second characteristic (e.g., sharing a common characteristic), but the first parameter (e.g., 100 tubes) is different than the second parameter (e.g., 200 tubes). Although one specific characteristic has been described, each cartridge 850, 852 can have multiple different parameters for each characteristic (e.g., common characteristic between the cartridges 850, 852).

As shown in FIGS. 33A and 33B, each cartridge 850, 852 can include a machine readable code 858, 860 (e.g., illustrated as a QR code), each of which can encode a unique cartridge identifier for each cartridge 850, 852. In some non-limiting examples, an imaging device of the system (e.g., the imaging device 815) can scan or otherwise acquire an image of the machine readable code, and the corresponding computing device can extract the cartridge identifier from the image or other image data of the machine readable code. In other cases, a computing device can receive a user input (e.g., from a user input device, such as a touchscreen) of the cartridge identifier (e.g., a user entering the unique alpha numeric number onto the touchscreen). As described above, each cartridge identifier can be associated with a list of characteristics, with each characteristic including a specific parameter (e.g., a database, a spreadsheet, a data table, etc.). In this way, particular characteristics and their underlying parameters can be easily extracted by a computing device when only the cartridge identifier is known.

As shown in FIGS. 33C and 33D, the cartridge 850 includes a first characteristic having a first parameter and a second characteristic having a second parameter. The first characteristic is the number of hollow tubes of the cartridge and the first parameter is the actual number of hollow tubes (i.e., 64 hollow tubes). The second characteristic is the number of segments of the hollow tubes to be actuated (or not to be actuated), while the second parameter is the number of or the specific segments of the hollow tubes to be actuated (or not to be actuated), such as, in this case, three segments to be actuated (and three segments not to be actuated). The cartridge 852 can include a third characteristic having a third parameter and a fourth characteristic having a fourth parameter. The third characteristic is the number of hollow tubes, which is the same as the first characteristic. However, the third parameter (e.g., the actual number of hollow tubes, in this case, 61 hollow tubes) is different than the first parameter. Similarly, the fourth characteristic is the number of segments of the hollow tubes to be actuated (or not be actuated), which is the same as the second characteristic). However, the fourth parameter (e.g., the actual segments, or number of segments to be actuated, in this case, is all the segments being actuated) is different than the second parameter (e.g., in which the “x” indicates the specific segments not to be actuated). Although these characteristics and corresponding parameters are just a few examples, the cartridges (and others) can include other shared characteristics with different parameters.

As described above, the variability between different patients can be quite high and may necessitate different parameters for different cartridges depending on the patient and other characteristics. For example, a patient characteristic can be wound/recipient size (e.g., the area of the wound). In this case, larger wounds can necessitate harvesting greater numbers of tissue portions (e.g., micrografts) and thus having a greater number of hollow tubes or actuatable segments of hollow tubes can be desirable. Conversely, smaller wounds require fewer numbers of tissue portions (e.g., corresponding to smaller numbers of tubes and fewer numbers of actuatable segments). As another example, a patient characteristic can be tissue thickness (e.g., skin thickness). As we age, the skin thickness decreases, and thus having extensions at an end of a hollow tube (e.g., the two triangular points at the distal end of each hollow tube defined by exterior bevels) that are shorter can correspond to the harvesting a thinner tissue portion (e.g., the axial length of a tissue portion). Correspondingly, the younger we are the thicker our skin tissue is, and thus having longer extensions at a distal end of a hollow tube can be desirable for harvesting thicker tissue portions. As yet another example, a patient characteristic can be harvest location or recipient site location (e.g., wound location). Some harvest locations or recipient site locations can have different curvatures, skin tissue thickness, etc., which can vary the required number of hollow tubes to be at a given location. As still yet another example, a patient characteristic can be tissue tightness (e.g., skin tightness), such as at the harvest location. Tighter skin can permit greater numbers of hollow tubes to penetrate the skin (e.g., because the skin is taught) and vice versa. Therefore, higher numbers of hollow tubes can be used for tighter skin, and vice versa. In some cases, some patient characteristics can be translated into other patient characteristics. For example, since age corresponds or is correlated with skin tissue thickness, a computing device receiving an input of age can generate the tissue thickness (e.g., from a table, formula, etc.) that links age for each tissue thicknesses. Similarly, since age corresponds or is correlated with tissue tightness, a computing device receiving an input of age can generate the tissue tightness. As another example, gender, race, ethnicity, etc., can correspond to particular patient characteristics such as, for example, tissue thickness, tissue tightness, etc., and thus a computing device receiving an input of gender, race, ethnicity, etc., can generate one or more corresponding patient characteristics.

Although the cartridges have been descried as having various characteristics with different parameters, in some cases, the system (e.g., the skin grafting system) can adjust operation of the system (e.g., the actuation system thereof), based on a received patient characteristics. For example, wound size is one type of patient characteristic described above, and thus after a computing device receives the patient characteristic (e.g., the specific wound size), the system 800 can adjust the operation of the system, based on this patient characteristic. In some cases, adjusting the operation of the system can include adjusting a default pattern of actuating segments of the plurality of hollow tubes (e.g., changing a maximum insertion distance of each segment, changing the number of segments to be actuated, adjusting the position of a pin within each hollow tube to accommodate larger or smaller tissue portions to be harvested, etc.). In some cases, the patient characteristic can be used to determine one or more operational parameters for the system 800 (e.g., which can be implemented by the computing device of the system 800). Similarly to the description above in this paragraph, the one or more operational parameters can be the maximum insertion distance of each segment, the number of hollow tubes to be actuated (e.g., the number of segments of the hollow tubes to be actuated), the pin position within each hollow tube, etc.

In some non-limiting examples, ensuring that cartridges are not reused is important for a number of reasons (e.g., to mitigate disease transmission, avoid blood from one patient interacting with another, etc.). Therefore, each cartridge can include a cartridge specific identifier, which is unique to that individual cartridge. In some cases, this cartridge specific identifier can be similar to the cartridge identifier, and thus can be an alphanumeric code, a numeric code, etc. Further, each unique cartridge specific identifier can be encoded by a corresponding machine readable code on the physical cartridge. For example, FIGS. 33A and 33B show each cartridge 850, 852 including a respective machine readable code 862, 864. More specifically, the machine readable code 862 can encode a first cartridge specific identifier unique to the cartridge 850, while the machine readable code 864 can encode a second cartridge specific identifier unique to the cartridge 852. In this way, a computing device can determine that a cartridge has (or has not) been reused. For example, a computing device (e.g., the computing device 808) can receive the cartridge specific identifier from the cartridge 850 (e.g., by scanning the machine readable code 862 with an imaging device to extract the encoded cartridge specific identifier). In some cases, the computing device can receive a user input (e.g., from a user input device, such as a display device) indicating that the cartridge has been coupled to a housing of a system (and thus is subsequently being used for a procedure. In other cases, the computing device can receive, from a sensor configured to sense coupling or decoupling of the cartridge from the housing, sensor information, a sensor value, etc., indicative of the cartridge being coupled to the cartridge. Regardless, the computing device can determine a time stamp associated with the cartridge specific identifier, such as when the computing device determines that the cartridge is coupled to the housing. Then, the computing device can store the cartridge specific identifier in memory (e.g., the memory of the computing device, or a different one, such as a server), and can store the time stamp associated with the cartridge specific identifier. After the procedure is completed, the computing device can determine that the cartridge has been decoupled from the housing (e.g., from a user input, or from a sensor). In this way, the computing device can store a first indication that the specific cartridge has been coupled to a housing previously (e.g., with a corresponding time stamp) and can store a second indication that the specific cartridge has been decoupled from the housing previously (e.g., with a corresponding time stamp), each of which can be associated with the cartridge specific identifier. This information can help mitigate falsely determining that the cartridge has been used when the cartridge can have simply been coupled and decoupled quickly. For example, the computing device can determine a time difference between the first indication and the second indication and can determine that the cartridge has not been used in a procedure, based on the time difference being smaller than a threshold time difference (e.g., indicating that the cartridge has simply been quickly attached and detached). Conversely, the computing device can determine that the cartridge has been used in a procedure, based on the time difference being greater than a threshold time difference).

In some non-limiting examples, a computing device can determine that a cartridge has been reused. For example, the computing device can receive a second cartridge specific identifier from a cartridge (e.g., by NFC, by scanning a machine readable code, etc.), can compare the second cartridge specific identifier to a first cartridge specific identifier (e.g., by querying a list of cartridge specific identifiers previously received), and can determine that the first cartridge specific identifier is the same as the second cartridge specific identifier, based on the comparison. If the computing device determines that the first cartridge specific identifier is not the same as the second cartridge specific identifier (e.g., they are different), a computing device can allow the system to implement a process (e.g., a harvesting process, such as allowing the plurality of hollow tubes to enter the tissue, a scattering process, etc.). If, however, the computing device determines that the first that the first cartridge specific identifier is the same as the second cartridge specific identifier, the computing device can prevent the system from implementing a process (e.g., preventing the plurality of hollow tubes from entering the tissue) and can transmit an alert, notification, etc., to a user. In some cases, the computing device can determine that the cartridge has been reused, based on the computing device determining that the first and second cartridge specific identifiers are the same, and the first specific cartridge identifier includes an associated first indication (e.g., indicating that the cartridge has been previously coupled to the housing). Further, the computing device can determine that the cartridge has been reused, based on the computing device determining that the first and second cartridge specific identifiers are the same, and the first specific cartridge identifier includes an associated first indication and a second indication (e.g., the second indication indicating that the cartridge has been previously decoupled to the housing). As yet a further example, the computing device can determine that the cartridge has been reused, based on the computing device determining that the first and second cartridge specific identifiers are the same, the first specific cartridge identifier includes an associated first indication and a second indication, and a time differential between the first identifier and the second identifier is greater than a threshold value.

In some non-limiting examples, when the computing device determines that the cartridge has been reused, the computing device can transmit an alert, a notification, etc., to a user indicating that the specific cartridge cannot be used in a subsequent procedure (e.g., a flashing red light). Further, when the computing device determines that the cartridge has been reused, the computing device can prevent the system from implementing a process (e.g., a harvesting process, a scattering process, moving a hollow tube, etc.).

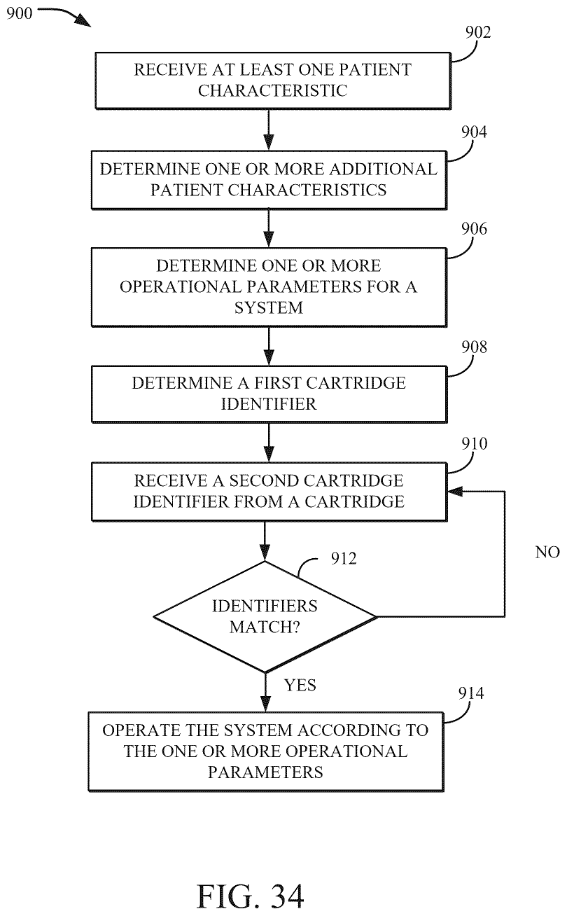

FIG. 34 shows a flowchart of a process 900 that can be implemented using any of the systems, devices, etc., described herein. Further, the process 900 can be implemented using one or more computing devices (e.g., the computing device 808). In some cases, the process 900 can be a process of validating or verifying a system (e.g., a skin grafting system), such as, prior to implementing a harvesting process or a scattering process. At 902, the process 900 can include receiving, using one or more computing devices, at least one patient characteristic.

At 904, the process 900 can include determining, using the one or more computing devices, one or more additional patient characteristics, based on the at least one patient characteristic. For example, as described above, some patient characteristics are correlated or otherwise linked to each other. In other words, one or more patient characteristics can be predictable to one or more other characteristics (e.g., by a known relationship between respective patient characteristics). As a more specific example, and as described above, a first patient characteristic that is age can be used, by the computing device (e.g., by using a known relationship between the characteristics, such as by using a table, a graph and corresponding functions, etc.), to determine a second patient characteristic that is extension height for one or more of the hollow tubes.

At 906, the process 900 can include determining, using the one or more computing devices, one or more operational parameters for a system (e.g., a skin grafting system, a handheld device, etc.), based on the at least one patient characteristics, the one or more additional patient characteristics, etc. As described above, the one or more operational parameters can be associated with a given patient characteristic. For example, a patient characteristic that is tissue thickness can be used to dictate the extension height for the one or more hollow tubes, such as, for example, by a computing device querying a database, a lookup table, etc. In some configurations, at least one operational parameter can be adjusted by the system during operation of the system. For example, maximum tube extension (e.g., the furthest a hollow tube is to be inserted into the tissue) can be adjusted by the system by the computing device advancing an actuator to only the maximum tube extension. In some non-limiting examples, at least one operational parameter cannot be adjusted by the system during operation of the system. For example, the extension height for the one or more hollow tubes (e.g., how far an extension having a sharp cutting edge extends from a distal end of a hollow tube) can be an operational parameter, but cannot be adjusted by the system (e.g., with a cartridge having those parameters). Therefore, the cartridge can include the at least one operational parameter that cannot be adjusted by the system during operation of the system. In some non-limiting examples, determining the one or more operational parameters can include adjusting or otherwise modifying one or more default operational parameters (e.g., that are able to be changed by the system, during operation of the system).

In some non-limiting examples, rather than determining the one or more operational parameters, the process 900 at block 906 can include receiving the one or more operational parameters, by, for example a user input from a user input device (e.g., a user entering the one or more operational parameters into the display device that is a touchscreen). In this case, therefore, blocks 902, 904, which are already indicated as being optional, can be omitted.

At 908, the process 900 can include determining, using the one or more computing devices, a first cartridge identifier, based on the one or more operational parameters. For example, different cartridges (e.g., identified by a respective cartridge identifier) having different operational parameters can be in table, database, etc. The one or more computing devices can then, using the one or more operational parameters, can match those operational parameters with those of a particular cartridge, to determine the first cartridge identifier (e.g., the first cartridge identifier being the cartridge that has the one or more operational parameters). In some cases, determining, using the one or more computing devices, the first cartridge identifier can be based on the at least one patient characteristic, the one or more additional patient characteristics, etc. For example, rather than using operational parameters to match the cartridge, each different cartridge along with its cartridge identifier can include one or more patient characteristics associated with the cartridge identifier. Therefore, the process 900 can include the one or more computing devices determining the first cartridge identifier by comparing the at least one patient characteristic (and the one or more additional patient characteristics) with one or more corresponding patient characteristics associated with a cartridge and its unique cartridge identifier (e.g., that has at least one, or all, the received or determined patient characteristics at blocks 902, 904 matching with the one or more corresponding patient characteristics associated with the cartridge identifier). In this case, the first cartridge identifier is the cartridge identifier associated with the cartridge.

At 910, the process 900 can include receiving, using the one or more computing devices, a second cartridge identifier from a cartage (e.g., the cartridge intended to be coupled to a housing of a system) As described above, the one or more computing devices can receive the second cartridge identifier in different ways, such as, for example, extracting the second cartridge identifier encoded by a machine readable code on the cartridge, receiving via a communication protocol (e.g., RFID, NFC, etc.).

At 912, the process 900 can include determining, using the one or more computing devices, that the first cartridge identifier is the same as the second cartridge identifier. If at 912, the one or more computing devices determine that the first cartridge identifier does not match with the second cartridge identifier, the process 900 can proceed back to block 910, in which the one or more computing devices can receive a different second cartridge identifier from a different cartridge. In some cases, the one or more computing devices can transmit a notification, an alert, etc., to a user indicating that the wrong cartridge is trying to be used, the cartridge identifiers do not match, etc., based on the one or more computing devices determining that the cartridge identifiers are not the same. If, however, at 912, the one or more computing devices determine that the first cartridge identifier is the same as the second cartridge identifier, the process 900 can proceed to block 914.

At 914, the process 900 can include operating, using the one or more computing devices, the system according to the one or more operational parameters (e.g., if the one or more operational parameters have been determined at the block 906). In some cases, this can include the one or more computing devices operating the system according to the one or more operational parameters that can be adjusted by the system during operation of the system. In some cases, this can include operating the system to implement a harvesting procedure, a scattering procedure, etc.

In some non-limiting examples, although the process 900 has been described as using patient characteristics, parameters, etc., to determine a cartridge identifier, in other configurations, the cartridge identifier can be used to determine the one or more operational parameters. For example, the one or more computing devices can receive a cartridge identifier from the cartridge (e.g., at the block 910) and can determine the one or more parameters, based on the cartridge identifier. More specifically, the one or more computing devices can compare the received cartridge identifier to a list, database, table, etc. (e.g., stored in the one or more computing devices or elsewhere) that has a cartridge identifier (e.g., a virtual one) that matches with the received cartridge identifier. Then, the one or more computing devices can determine the one or more parameters to be those associated with the cartridge identifier (e.g., that matches with the received cartridge identifier). These operational parameters can then be used to operate the system (e.g., the skin grafting system).

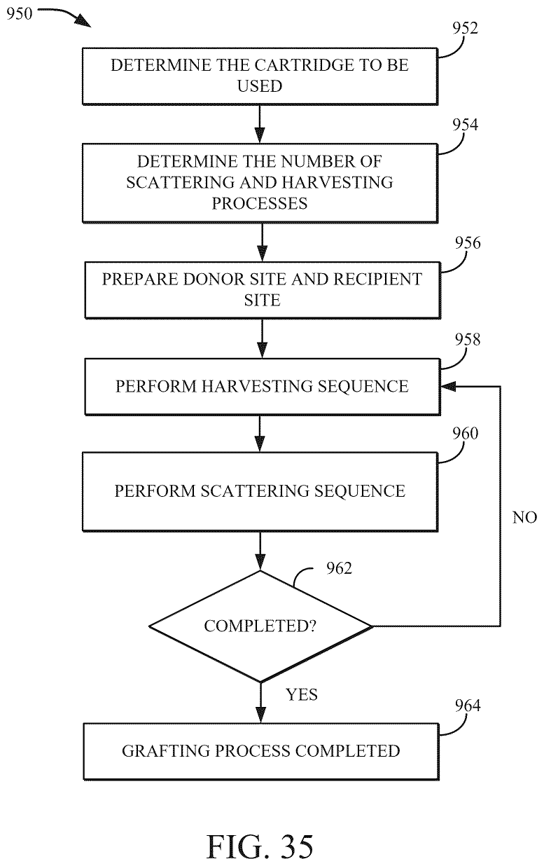

FIG. 35 shows a flowchart of a process 950 of tissue grafting, and particularly, autologous tissue grafting. The process 950 can be implemented using any of the systems, devices, etc., described herein. Further, the process 950 can be implemented using one or more computing devices (e.g., the computing device 808).

At 952, the process 950 can include determining, using one or more computing devices, a cartridge to be used. In some cases, a practitioner can determine the type of cartridge to be used, and this can include the one or more computing devices determining that the type of cartridge to be used (e.g., via receiving a user input indicative of the type of cartridge to be used). In some cases, this can include one or more blocks of the process 900 to, for example, validate the cartridge (sensed by the system) is permitted to be used (e.g., matches with the determined cartridge to be used, has not been reused, etc.).

At 954, the process 950 can include determining, using the one or more computing devices, the number of scattering and harvesting sequences. In some cases, this block 954 can be implemented in a similar manner as the description with respect to FIGS. 32A and 32B. In some cases, determining the number of scattering and harvesting sequences can be based on the determined cartridge to be used at block 952 (e.g., the number of hollow tubes in the cartridge, the density of the hollow tubes, etc.).

At 956, the process 950 can include preparing the donor site and the recipient site. In some cases, this can include cleaning, and anesthetizing or otherwise numbing the donor site (e.g., a skin tissue site). This can also include prepping the recipient site (e.g. cleaning the recipient site). For example, when the recipient site is includes a scar, a practitioner can remove some or all of the scar so as to permit seeding of the deposited tissue portions when placed on the recipient site.

At 958, the process 950 can include performing, using the one or more computing devices a harvesting sequence, which can be implemented using any of the previous harvesting devices, systems, methods, etc., described above. Once the harvesting sequence has been completed, the one or more computing devices can decrease the number of harvesting sequences determined at 954 by one. This decreased number of harvesting sequences can be presented to a user by the one or more computing devices.

At 960, the process 950 can include performing, using the one or more computing devices a scattering sequence, which, again, can be implemented using any of the previous scattering devices, systems, methods, etc., described above. Once the scattering sequence has been completed, the one or more computing devices can decrease the number of scattering sequences determined at 954 by one. This decreased number of scattering sequences can be presented to a user by the one or more computing devices.

At 962, the process 950 can include determining, using the one or more computing device, whether the number of harvesting and scattering processes have been completed. If at block 962, the one or more computing devices determine that the number of harvesting and scattering processes have not be completed (e.g., where the decreased number of harvesting and scattering processes each have a number greater than zero), the process 950 can proceed back to the block 958 to prompt a practitioner to perform an additional harvesting sequence. If, however, at the block 962, the one or more computing devices determine that the number of harvesting and scattering processes have been completed (e.g., where the current number of harvest and scattering processes each is zero), the one or more computing device can determine that grafting is complete and the process 950 can proceed to the block 964.

At 964, the process 950 can include the completion of the grafting process. In some cases, once completed, a user can remove the cartridge from the housing of the system. At this point, the one or more computing device can add the cartridge identifier of this cartridge (e.g., determined at the block 952) to a list of previously used cartridges. In this way, once the cartridge is removed, it advantageously cannot be reused, so as to prevent inadvertently using a used cartridge for a different subject (e.g., person). In some cases, once completed, the one or more computing devices can prompt the user to recharge the battery pack (e.g., by placing the system on the charging dock).

Although the description and corresponding drawings have detailed some systems as including a computing device implementing some or all of the corresponding process steps, in some configurations, the system can include multiple computing devices, each of which can implement some or all of the process steps. In some cases, one computing device can be external to a housing of the system (e.g., a handheld device).

The present disclosure has described one or more preferred non-limiting examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single-or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications can be made to these configurations without departing from the scope or spirit of the claimed subject matter.

Certain operations of methods according to the disclosure, or of systems executing those methods, can be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component can be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, can be localized on one computer, can be distributed between two or more computers or other processor devices, or can be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions can be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, ±5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

Various features and advantages of the disclosure are set forth in the following claims.

While the present disclosure can be susceptible to various modifications and alternative forms, specific configurations have been shown by way of example in the drawings and have been described in detail herein. However, it should be understood that the present disclosure is not intended to be limited to the particular forms disclosed. Rather, the present disclosure is to cover all modifications, equivalents, and alternatives falling within the spirit and scope of the present disclosure as defined by the following appended claims.

This written description uses examples to disclose the present disclosure, including the best mode, and also to enable any person skilled in the art to practice the present disclosure, including making and using any devices or systems and performing any incorporated methods. The patentable scope of the present disclosure is defined by the claims and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Finally, it is expressly contemplated that any of the processes or steps described herein can be combined, eliminated, or reordered. Accordingly, this description is meant to be taken only by way of example, and not to otherwise limit the scope of this present disclosure.

What is claimed is:

1. A system comprising:
a plurality of hollow tubes, each hollow tube of the plurality of hollow tubes being configured to harvest a respective tissue portion from a tissue site, wherein the respective tissue portion includes skin tissue;
an actuation system configured to translate the plurality of hollow tubes simultaneously into the tissue site; and
a rotational drive configured to rotate each hollow tube of the plurality of tubes; and
wherein at least one of:
as the actuation system translates the plurality of hollow tubes into the tissue site, the rotational drive is configured to rotate each hollow tube of the plurality of hollow tubes; or
after the actuation system translates the plurality of hollow tubes into the tissue site, the rotational drive is configured to rotate each hollow tube of the plurality of hollow tubes to ensure each tissue portion is severed from the tissue site.

2. The system of claim 1, wherein the rotational drive rotates each hollow tube of the plurality of hollow tubes about a longitudinal axis of the respective hollow tube.

3. The system of claim 1, wherein the plurality of hollow tubes is a first plurality of hollow tubes and further comprising a second plurality of hollow tubes;
wherein the actuation system is configured to independently translate the first plurality of hollow tubes and the second plurality of hollow tubes; and
wherein the rotational drive is configured to rotate each hollow tube of the second plurality of hollow tubes.

4. The system of claim 3, further comprising an array of hollow tubes that includes the first plurality of hollow tubes and the second plurality of hollow tubes.

5. The system of claim 4, wherein the first plurality of hollow tubes is a first row of hollow tubes; and
wherein the second plurality of hollow tubes is a second row of hollow tubes.

6. The system of claim 1, wherein the rotational drive is at least one of a belt or a chain.

7. The system of claim 1, wherein an inner width of each hollow tube of the plurality of hollow tubes is less than or equal to 1 millimeter; or
wherein an inner width of each hollow tube of the plurality of hollow tubes is less than or equal to 0.6 millimeters.

8. A system comprising:
a first plurality of hollow tubes, each hollow tube of the first plurality of hollow tubes being configured to harvest a first respective tissue portion from a tissue site, wherein the first respective tissue portion includes skin tissue;
a second plurality of hollow tubes, each hollow tube of the second plurality of hollow tubes being configured to harvest a second respective tissue portion from the tissue site, wherein the second respective tissue portion includes skin tissue;
an actuation system configured to independently translate the first plurality of hollow tubes and the second plurality of hollow tubes; and
wherein the actuation system includes:
a spring;
a plunger coupled to the spring;
a motor coupled to the plunger and configured to load the spring;
a brake configured to lock the plunger to prevent unloading of the spring; and
wherein the brake is configured to release the plunger to allow the spring to unload and drive the plunger thereby forcing the first plurality of hollow tubes into the tissue site.

9. The system of claim 8, wherein the motor is configured to move the plunger back into a first position thereby reloading the spring;
wherein when the plunger is in the first position, the brake is configured to lock the plunger into the locked position; and
wherein when the plunger is in the first position with the spring reloaded, the brake is configured to release the plunger thereby unloading the spring to force the second plurality of hollow tubes into the tissue site.

10. The system of claim 8, further comprising:
a housing;
a battery pack electrically coupled to the actuation system and removably coupled to the housing; and
wherein the motor is a DC motor.

11. A system comprising:
a housing;
a plurality of hollow tubes, each hollow tube of the plurality of hollow tubes being configured to harvest a respective tissue portion from a tissue site, wherein the respective tissue portion includes skin tissue;
an image sensor coupled to the housing and configured to acquire one or more images of the tissue site or other surrounding area;
a display; and
one or more computing devices in communication with the image sensor and the display, the one or more computing devices being configured to:
acquire, using the image sensor, an image of at least a portion of the tissue site; and
cause the display to present the image of the portion of the tissue site.

12. The system of claim 11, wherein the plurality of hollow tubes is a first plurality of hollow tubes and further comprising:
a second plurality of hollow tubes, each hollow tube of the second plurality of hollow tubes being configured to harvest a respective tissue portion from the tissue site that includes skin tissue; and
an actuation system configured to independently translate the first plurality of hollow tubes and the second plurality of hollow tubes.

13. The system of claim 11, wherein at least one of:
the display is coupled to the housing;
the image sensor is coupled to a first end of the housing opposite a second end of the housing that includes a handle; or
the one or more computing devices are further configured to shift the image of the at least the portion of the tissue site, based on the distance between the image sensor and a center of the plurality of hollow tubes.

14. A system comprising:
a first plurality of hollow tubes, each hollow tube of the first plurality of hollow tubes being configured to harvest a first respective tissue portion from a tissue site, wherein the first respective tissue portion includes skin tissue;
a second plurality of hollow tubes, each hollow tube of the second plurality of hollow tubes being configured to harvest a second respective tissue portion from the tissue site, wherein the second respective tissue portion includes skin tissue;
a peripheral housing that surrounds the first plurality of hollow tubes and the second plurality of hollow tubes;
a stabilizer coupled to the peripheral housing, the stabilizer separating the first plurality of hollow tubes from the second plurality of hollow tubes; and
wherein the peripheral housing and the stabilizer are configured to be forced against the tissue site to stabilize the tissue site during insertion of the first plurality of hollow tubes or the second plurality of hollow tubes into the tissue site.

15. The system of claim 14, further comprising an actuation system configured to independently translate the first plurality of hollow tubes and the second plurality of hollow tubes.

16. The system of claim 14, wherein the stabilizer is a first stabilizer and further comprising:
a third plurality of hollow tubes, each hollow tube of the third plurality of hollow tubes being configured to harvest a third respective tissue portion from a tissue site that includes skin tissue; and
a second stabilizer coupled to the peripheral housing, the second stabilizer separating the second plurality of hollow tubes from the third plurality of hollow tubes.

17. The system of claim 14, further comprising:
a device housing; and
a cartridge removably coupled to the device housing, the cartridge including the peripheral housing, the stabilizer, the first plurality of hollow tubes, and a second plurality of hollow tubes.

18. The system of claim 14, wherein the stabilizer is rigid;
wherein the stabilizer is substantially flat; and
wherein when the stabilizer is forced against the tissue site, the tissue site flattens against the stabilizer.

19. A system comprising:
a housing;
a plurality of hollow tubes, each hollow tube of the plurality of hollow tubes being configured to harvest a respective tissue portion from a tissue site and subsequently dispense the respective tissue portion onto a target site, wherein the respective tissue portion includes skin tissue;
an image sensor coupled to the housing and configured to acquire one or more images of the tissue site;
a light source coupled to the housing; and
one or more computing devices in communication with the image sensor and the light source, the one or more computing devices being configured to at least one of:
cause the light source to illuminate the target site during acquisition of an image of the target site by the image sensor; or
cause the light source to project an illumination pattern at the target site, the position of the illumination pattern corresponding with a scattering location of the tissue portions from the plurality of hollow tubes onto the tissue site.

20. A system comprising:
a housing;
a plurality of hollow tubes, each hollow tube of the plurality of hollow tubes being configured to harvest a respective tissue portion from a tissue site and subsequently dispense the respective tissue portion onto a target site, wherein the respective tissue portion includes skin tissue;
a scattering system configured to dispense the tissue portion from each hollow tube of the plurality of hollow tubes onto the target site; and
a substrate coupled to or removably coupled to the housing, the substate being adjustable to adjust a distance between the plurality of hollow tubes and the target site; and
wherein a portion of the substate contacts the target site when the scattering system dispenses the portions of tissue from the plurality of hollow tubes.
